SEQUENCE LISTING (7 sequences)

SEQ ID NO: 1 - DNA, 14553 bp, synthetic construct
  misc_feature 1..14553: note = Synthetic:pVSV-deltaG-NiVB G
  source 1..14553: mol_type = other DNA
acgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtctgttcacgcgtggtaccttcatcatgccgacagaaagcaagaaagttagattcgaaaatactgcttcagacaaggggaaaaatcctagtaaagttattaagagctactacggaaccatggacattaagaaaataaatgaagggttattggacagcaagatattaagtgctttcaacacagtgatagcactgcttggatccattgtaatcatagtgatgaatataatgatcatccaaaactacacaagatcaacagataatcaggccatgatcaaagatgcattgcagagtatccagcagcagatcaaggggcttgccgacaaaattggcacagagatagggccgaaagtatcactgattgatacatccagtactatcactatcccagctaatattgggctgttaggttcaaagatcagccagtcaactgcaagtataaatgagaatgtgaatgaaaaatgcaaatttacactgcctcccttgaaaatccacgaatgtaacatttcttgtcctaacccactcccttttagagagtataagccgcagacagaaggagtgagcaatctggtaggattacctaataatatctgtctgcaaaagacatctaatcagatactgaaaccaaagctgatttcatacaccttacccgtagtcggtcaaagtggcacctgtatcacagacccactgctggctatggatgagggctactttgcatatagccacctggaaaaaatcggatcatgttcaagaggggtctccaaacaaagaataataggagttggagaggtactagacagaggtgacgaagtaccttctttgtttatgactaacgtctggaccccatcaaatccaaacaccgtttaccattgcagtgccgtgtacaacaatgaattctattatgtgctttgtgcagtgtcagttgttggagaccctattctgaatagcacctactggtccggatcactaatgatgactcgtctagctgtaaaacctaagaataatggtgagagttacaatcaacatcaatttgccttacggaatattgagaaagggaagtatgataaagttatgccatatggaccctcaggcatcaaacaaggtgacaccctgtactttcctgctgtaggatttttggtcaggacagagttcaaatacaatgattcaaattgtcccatcgcagagtgtcaatacagcaaacctgaaaactgcaggctatctatggggattagaccaaacagtcattatatccttcgatctggactactaaaatacaatctatcggatgaggagaactctaaaattgtattcattgaaatatctgatcaaagactatctattggatctcctagcaaaatctatgattctttgggtcaacctgttttctaccaagcatctttttcatgggacactatgattaaatttggagatgtccaaacagttaaccctttagttgtaaattggcgtgacaacacggtaatctcaagacctgggcaatcacaatgccctagattcaacaagtgcccagaggtttgctgggaaggggtttataatgatgccttcctgattgatagaatcaattggataagcgcgggtgtattccttgacagcaaccagaccgcagagaatcctgtttttactgtattcaaagataatgaagtactttacagagcacaactagcttccgaggacaccaatgcacaaaaaacaataactaattgcttccttttgaagaataagatctggtgtatatcactggttgagatatacgacacaggagacaatgttataagacctaaactattcgcagttaagataccagagcaatgtacataacctaggctagggctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg

SEQ ID NO: 2 - DNA, 11456 bp, synthetic construct
  misc_feature 1..11456: note = Synthetic: rVSV-deltaG-NiVB G
  source 1..11456: mol_type = other DNA
acgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtctgttcacgcgtggtaccttcatcatgccgacagaaagcaagaaagttagattcgaaaatactgcttcagacaaggggaaaaatcctagtaaagttattaagagctactacggaaccatggacattaagaaaataaatgaagggttattggacagcaagatattaagtgctttcaacacagtgatagcactgcttggatccattgtaatcatagtgatgaatataatgatcatccaaaactacacaagatcaacagataatcaggccatgatcaaagatgcattgcagagtatccagcagcagatcaaggggcttgccgacaaaattggcacagagatagggccgaaagtatcactgattgatacatccagtactatcactatcccagctaatattgggctgttaggttcaaagatcagccagtcaactgcaagtataaatgagaatgtgaatgaaaaatgcaaatttacactgcctcccttgaaaatccacgaatgtaacatttcttgtcctaacccactcccttttagagagtataagccgcagacagaaggagtgagcaatctggtaggattacctaataatatctgtctgcaaaagacatctaatcagatactgaaaccaaagctgatttcatacaccttacccgtagtcggtcaaagtggcacctgtatcacagacccactgctggctatggatgagggctactttgcatatagccacctggaaaaaatcggatcatgttcaagaggggtctccaaacaaagaataataggagttggagaggtactagacagaggtgacgaagtaccttctttgtttatgactaacgtctggaccccatcaaatccaaacaccgtttaccattgcagtgccgtgtacaacaatgaattctattatgtgctttgtgcagtgtcagttgttggagaccctattctgaatagcacctactggtccggatcactaatgatgactcgtctagctgtaaaacctaagaataatggtgagagttacaatcaacatcaatttgccttacggaatattgagaaagggaagtatgataaagttatgccatatggaccctcaggcatcaaacaaggtgacaccctgtactttcctgctgtaggatttttggtcaggacagagttcaaatacaatgattcaaattgtcccatcgcagagtgtcaatacagcaaacctgaaaactgcaggctatctatggggattagaccaaacagtcattatatccttcgatctggactactaaaatacaatctatcggatgaggagaactctaaaattgtattcattgaaatatctgatcaaagactatctattggatctcctagcaaaatctatgattctttgggtcaacctgttttctaccaagcatctttttcatgggacactatgattaaatttggagatgtccaaacagttaaccctttagttgtaaattggcgtgacaacacggtaatctcaagacctgggcaatcacaatgccctagattcaacaagtgcccagaggtttgctgggaaggggtttataatgatgccttcctgattgatagaatcaattggataagcgcgggtgtattccttgacagcaaccagaccgcagagaatcctgtttttactgtattcaaagataatgaagtactttacagagcacaactagcttccgaggacaccaatgcacaaaaaacaataactaattgcttccttttgaagaataagatctggtgtatatcactggttgagatatacgacacaggagacaatgttataagacctaaactattcgcagttaagataccagagcaatgtacataacctaggctagggctagccagattcttcatgtttggaccaaatcaacttgtgataccatgctcaaagaggcctcaattatatttgagtttttaatttttatgaaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 3 - protein, 422 aa, synthetic construct
  REGION 1..422: note = Synthetic: VSV N protein sequence
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 4 - protein, 265 aa, synthetic construct
  REGION 1..265: note = Synthetic: VSV P protein sequence
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 5 - protein, 229 aa, synthetic construct
  REGION 1..229: note = Synthetic: VSV M protein sequence
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 6 - protein, 602 aa, synthetic construct
  REGION 1..602: note = Synthetic: NiVB G protein sequence
MPTESKKVRFENTASDKGKNPSKVIKSYYGTMDIKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFKYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 7 - protein, 2109 aa, synthetic construct
  REGION 1..2109: note = Synthetic: VSV L protein sequence
MEVHDFETDEFNDFNEDDYATREFLNPDERMTYLNHADYNLNSPLISDDIDNLIRKFNSLPIPSMWDSKNWDGVLEMLTSCQANPISTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

(19) United States
(12) Patent Application Publication
(10) Pub. No.: US 2025/0127878 A1
(43) Pub. Date: Apr. 24, 2025

(54) Rapid Acting Vaccine Against Nipah Virus

(71) Applicant: Board of Regents, The University of Texas System, Austin, TX (US)

(72) Inventors: Stephanie L. Foster, Atlanta, GA (US); Chad E. Mire, Galveston, TX (US); Robert W. Cross, Galveston, TX (US); Thomas W. Geisbert, Albany, TX (US)

(21) Appl. No.: 18/693,225

(22) PCT Filed: Sep. 22, 2022
(86) PCT No.: PCT/US2022/076850

§ 371 Date: Mar. 19, 2024

Related U.S. Application Data
(60) Provisional application No. 63/248,055, filed on Sep. 24, 2021.

(51) Int. Cl. A61K 39/155; A61K 39/00; A61P 31/14; C12N 7/00; C12N 15/86

(52) U.S. Cl. CPC A61K 39/155; A61P 31/14; C12N 7/00; C12N 15/86; A61K 2039/5256; C12N 2760/18222; C12N 2760/18234; C12N 2760/20222; C12N 2760/20243

(57) ABSTRACT

The present invention includes methods of making, compositions, or vaccinations comprising a recombinant vesicular stomatitis vims (rVSV) viral vector that expresses a Nipah Virus protein antigen, wherein the rVSV vector comprises one or more heterologous polynucleotides coding for and expressing a Nipah Virus NiV_{B} G-protein antigen; wherein the NiV_{B} G-protein comprises an amino acid sequence as set forth in SEQ ID NO: 6, or wherein the heterologous polynucleotide encodes a. polypeptide coding for the NiV_{B} G-protein antigen comprising at least 90% sequence identity to an amino acid sequence as set forth in SEQ ID NO: 6, or 90% sequence identity to a nucleic acid sequence as set forth in SEQ ID NO: 2, wherein the composition or vaccine is effective to reduce or prevent a Nipah Virus infection at 3 days.

### CROSS-REFERENCE TO RELATED APPLICATIONS

This application is a National Stage of International Application No. PCT/US2022/076850, filed Sep. 22, 2022, which claims the benefit of U.S. Provisional Application No. 63/248,055, filed Sep. 24, 2021. The contents of each of which are incorporated by reference in their entirety.

### STATEMENT OF FEDERALLY FUNDED RESEARCH

This invention was made with government support under W81XWH1910028 awarded by the Department of Defense. The government has certain rights in the invention.

### TECHNICAL FIELD OF THE INVENTION

The present invention relates in general to the field of vaccines against viruses, and more particularly, to a Rapid acting vaccine against Nipah virus.

### INCORPORATION-BY-REFERENCE OF MATERIALS FILED ON COMPACT DISC

The present application includes a Sequence Listing which has been submitted in ST.26 XML format via EFS-Web and is hereby incorporated by reference in its entirety. Said ST.26 XML copy, created on Sep. 15, 2022, is named UTMB2032WO.xml and is 39, kilo bytes in size.

### BACKGROUND OF THE INVENTION

Without limiting the scope of the invention, its background is described in connection with Nipah virus.

In 1998, Nipah virus (NiV) emerged and was shown to be a previously unknown paramyxovirus, now classified along with Hendra virus (HeV) and Cedar virus within the Henipavirus genus. NiV causes febrile encephalitis and severe respiratory disease in humans, with a case-fatality rate (CFR) as high as 100% in some outbreaks. Outbreaks of NiV have occurred almost annually in Bangladesh and India from 2001 to 2020, and a 2018 outbreak in India caused 17 deaths out of 19 cases. Genetic analysis has identified at least two strains of NiV responsible for outbreaks in different geographical areas. The Malaysia strain (NiV_{M}) caused the initial outbreak of NiV from 1998-1999 in Malaysia and Singapore in which over 270 people were infected with a CFR of about 40%, and an additional 2014 outbreak in the Philippines had a CFR of approximately 52%. The Bangladesh strain (NiV_{B}), however, has caused outbreaks of NiV_{B} associated with person-to-person transmission, including in healthcare settings, and with higher CFRs, averaging about 75%. Importantly, the inventors have developed nonhuman primate (NHP) models for both NiV_{M}¹ and NiV_{B}² and recently showed that NiV_{B} is more pathogenic in African green monkeys (AGMs) than NiV_{M} under identical experimental conditions². The inventors also showed that treatments that protect AGMs against NiV_{M} were not as effective against NiV_{B}², demonstrating the importance of any medical countermeasure to protect against the ongoing public health threat of the more virulent NiV_{B}.

NiV has a negative-sense, single-stranded RNA genome comprising six genes: N, P, M, F, G and L. The F gene encodes the fusion protein, which allows for fusion of the viral and host cell membranes during viral entry. The G gene encodes the glycoprotein, which is the viral attachment protein that recognizes host ephrin-B2 and ephrin-B3 receptors expressed on endothelial cells and neurons that are highly conserved among many mammalian species. Vaccine studies in pre-clinical animal models of NiV disease have focused primarily on the F and G proteins as antigens.

Currently, there are no vaccines licensed for the prevention of NiV disease. What is needed is a highly effective vaccine or immunization that acts rapidly to provide immunity against all variants, and in particular, the highly pathogenic NiV_{B}.

### SUMMARY OF THE INVENTION

In one embodiment, the present invention includes a composition or vaccine comprising a recombinant vesicular stomatitis virus (rVSV) viral vector that expresses a Nipah Virus protein, wherein the rVSV vector comprises one or more heterologous polynucleotides coding for and expressing a Nipah Virus NiV_{B} G-protein; wherein the NiV_{B} G-protein antigen comprises an amino acid sequence as set forth in SEQ ID NO: 6, or wherein the heterologous polynucleotide encodes a polypeptide coding for the NiV_{B} G-protein antigen comprising at least 90% sequence identity to an amino acid sequence as set forth in SEQ ID NO: 6, or 90% sequence identity to a nucleic acid sequence as set forth in SEQ ID NO: 2, wherein the composition or vaccine is effective to reduce or prevent a Nipah Virus infection at 3 days against both Malaysia strain (NiV_{M}) and Bangladesh strain (NiV_{B}). In one aspect, the polynucleotide has a 91, 92, 93, 94, 95, 96, 97, 98, 99, or 100% sequence identity to the nucleic acid sequence as set forth in SEQ ID NO: 2. In another aspect, the polynucleotide has a 91, 92, 93, 94, 95, 96, 97, 98, 99, or 100% sequence identity to the sequence as set forth in SEQ ID NO: 6. In another aspect, the composition or vaccine is effective to reduce or prevent a Nipah Virus infection with a single dose at 3 days. In another aspect, the polynucleotide encoding the NiV_{B} G-protein antigen is operably linked to a promoter selected from the group consisting of an immediate early cytomegalovirus (CMV) promoter, guinea pig CMV promoter, an SV40 promoter, Human Herpesvirus Type III glycoprotein B (HHV3gB) promoter, Pseudorabies Virus promoters, glycoprotein X promoter, Herpes Simplex Virus-1 alpha 4 promoter, a Marek's Disease Virus glycoprotein A (or gC) promoter, a Marek's Disease Virus glycoprotein B promoter, a Marek's Disease Virus glycoprotein E promoter, a Marek's Disease Virus glycoprotein I promoter, an Infectious Laryngotracheitis Virus glycoprotein B, an Infectious Laryngotracheitis Virus glycoprotein E promoter, an Infectious Laryngotracheitis Virus glycoprotein D promoter, an Infectious Laryngotracheitis Virus glycoprotein I promoter, vaccinia H6, and a combination thereof. In another aspect, the polynucleotide encoding the NiV_{B} G-protein is inserted between a VSV-M protein and a VSV-L protein on a VSV genome. In another aspect, the composition or vaccine further comprises a pharmaceutically or veterinarily acceptable carrier, excipient, vehicle or adjuvant. In another aspect, the composition or vaccine does not comprise a Green Fluorescent Protein protein or gene. In another aspect, an immune response occurs within 3 days and provides protection at 7 days for Malaysian Nipah, Bangladesh Nipah, or both.

In another embodiment, the present invention includes a method of vaccinating an animal or for inducing an immunogenic or protective response in an animal against avian influenza pathogens, comprising at least one administration of the composition comprising a recombinant vesicular stomatitis virus (rVSV) viral vector that expresses a Nipah Virus protein, wherein the rVSV vector comprises one or more heterologous polynucleotides coding for and expressing a Nipah Virus NiV_{B} G-protein; wherein the NiV_{B} G-protein antigen comprises an amino acid sequence as set forth in SEQ ID NO: 6, or wherein the heterologous polynucleotide encodes a polypeptide coding for the NiV_{B} G-protein antigen comprising at least 90% sequence identity to an amino acid sequence as set forth in SEQ ID NO: 6, or 90% sequence identity to a nucleic acid sequence as set forth in SEQ ID NO: 2, wherein the composition or vaccine is effective to reduce or prevent a Nipah Virus infection at 3 days against both Malaysia strain (NiV_{M}) and Bangladesh strain (NiV_{B}). In one aspect, the polynucleotide has a 91, 92, 93, 94, 95, 96, 97, 98, 99, or 100% sequence identity to the nucleic acid sequence as set forth in SEQ ID NO: 2. In another aspect, the polynucleotide has a 91, 92, 93, 94, 95, 96, 97, 98, 99, or 100% sequence identity to the amino acid sequence as set forth in SEQ ID NO: 6. In another aspect, the Nipah Virus is a Malaysia strain (NiV_{M}), or a Bangladesh strain (NiV_{B}). In another aspect, the composition or vaccine is effective to reduce or prevent a Nipah Virus infection with a single dose at 3 days. In another aspect, the polynucleotide encoding the NiV_{B} G-protein antigen is operably linked to a promoter selected from the group consisting of an immediate early cytomegalovirus (CMV) promoter, guinea pig CMV promoter, an SV40 promoter, Human Herpesvirus Type III glycoprotein B (HHV3gB) promoter, Pseudorabies Virus promoters, glycoprotein X promoter, Herpes Simplex Virus-1 alpha 4 promoter, a Marek's Disease Virus glycoprotein A (or gC) promoter, a Marek's Disease Virus glycoprotein B promoter, a Marek's Disease Virus glycoprotein E promoter, a Marek's Disease Virus glycoprotein I promoter, an Infectious Laryngotracheitis Virus glycoprotein B, an Infectious Laryngotracheitis Virus glycoprotein E promoter, an Infectious Laryngotracheitis Virus glycoprotein D promoter, an Infectious Laryngotracheitis Virus glycoprotein I promoter, vaccinia H6, and a combination thereof. In another aspect, the polynucleotide encoding the NiV_{B} G-protein is inserted between a VSV-M protein and a VSV-L protein on a VSV genome. In another aspect, the composition or vaccine further comprises a pharmaceutically or veterinarily acceptable carrier, excipient, vehicle or adjuvant. In another aspect, an immune response occurs within 3 days and provides protection at 7 days for Malaysian Nipah, Bangladesh Nipah, or both. In another aspect, the administration further comprises a prime-boost administration regimen. In another aspect, the animal is a human. In another aspect, the composition or vaccine does not comprise a Green Fluorescent Protein gene.

In another embodiment, the present invention includes a recombinant viral vector composition or vaccine comprising a recombinant vesicular stomatitis virus (rVSV) viral vector that expresses a Nipah Virus protein, wherein the rVSV vector comprises one or more heterologous polynucleotides coding for and expressing a Nipah Virus NiV_{B} G-protein antigen; wherein the NiV_{B} G-protein antigen comprises an amino acid sequence as set forth in SEQ ID NO: 6, or wherein the heterologous polynucleotide encodes a polypeptide coding for the NiV_{B} G-protein antigen comprising at least 90% sequence identity to the sequence as set forth in SEQ ID NO: 6, or 90% sequence identity to an nucleic acid sequence as set forth in SEQ ID NO: 2, wherein the composition or vaccine is effective to reduce or prevent a Nipah Virus infection at 3 days.

### BRIEF DESCRIPTION OF THE DRAWINGS

For a more complete understanding of the features and advantages of the present invention, reference is now made to the detailed description of the invention along with the accompanying FIG.s and in which:

FIG. 1A is a schematic of the rVSV-ΔG-NiV_{B} G-GFP RNA genome with the name of each gene indicated (VSV N, VSV P. VSV M, NiV_{B} G, GFP, and VSV L).

FIG. 1B is a schematic of the rVSV-ΔG-NiV_{B} G RNA genome with the name of each gene indicated (VSV N, VSV P, VSV, M, NiV_{B} G, and VSV L). For both FIGS. 1A and 1B, the genes are shown as boxes, while the intergenic regions and 3′ and 5′ untranslated regions are shown as black lines. VSV genes are shown in blue, the NiV_{B} G gene is shown in pink, and the GFP gene is shown in green. The 3′ and 5′ ends of the negative-sense viruses are indicated. GFP, green fluorescent protein; NiV_{B}, Nipah virus Bangladesh strain; RNA, ribonucleic acid; rVSV, recombinant vesicular stomatitis virus; VSV, vesicular stomatitis virus.

FIGS. 2A and 2B shows the survival curves following challenge of vaccinated AGMs with NiV_{B}. Kaplan-Meier survival curves for AGMs vaccinated with rVSV-dG-NiV_{B} G (blue line) or rVSV-dG-EBOV 76 (red line)(A) seven days or (B) three days prior to challenge with NiV_{B}. AGM, African green monkey; EBOV, Zaire ebolavirus; NiV_{B}, Nipah virus Bangladesh strain; rVSV, recombinant vesicular stomatitis virus.

FIG. 3: Recovery of rVSV-ΔG-NiV_{B} G virions. Diagram of the process by which recovery of rVSV-ΔG-NiV_{B} G virions occurs in BHK-21 clone WI-2 cells. 1. Cells are infected with vTF7-3 to produce bacteriophage T7 polymerase. 2. Cells are transfected with the full-length pVSV-ΔG-NiV_{B} G plasmid and helper plasmids encoding VSV G, VSV N, VSV P, and VSV L (all under the control of the T7 promoter). 3. The helper plasmids are transcribed and translated to produce VSV N, P, and L protein; the full-length plasmid is transcribed to produce the RNA genome of rVSV-ΔG-NiV_{B} G. Together, they form the RNP complex. 4. The RNA genome is transcribed to produce mRNAs encoding VSV N, P, M, and L and NiV_{B} G. 5. The mRNAs are translated to produce VSV N, P, M and L and NiV_{B} G proteins. 6. The RNA genome and viral structural proteins are packaged into virions near the cell membrane. 7. Live virions bud out from the cell with NiV_{B} G protein on their surface. BHK, baby hamster kidney; mRNA, messenger ribonucleic acid; NiV_{B}, Nipah virus Bangladesh strain, RNA, ribonucleic acid; RNP, ribonucleoprotein; rVSV, recombinant vesicular stomatitis virus; VSV, vesicular stomatitis virus. Figure created using BioRender.com.

FIGS. 4A to 4K: The rVSV-ΔG-NiV_{B} G construct expresses NiV_{B} G protein. Results of an IFA in BHK-21 clone WI-2 (panels A through E) and Vero 76 cells (panels F through K) infected with G*-rVSV-ΔG-NiV_{B} G (panels A, B, D, E, G, H, J, and K) or mock-infected with growth medium only (panels C, F, and I). Cells were permeabilized with 0.5% Triton™ X-100 (panels B and C and F through H) or not permeabilized (panels C through E and I through K) and then incubated with human m102.4 primary antibody (panels B, C, E, F, H, I, and K) followed by a secondary antibody against human IgG conjugated to a green fluorescent fluorophore (all panels). BHK, baby hamster kidney; IFA, immunofluorescence assay; IgG, immunoglobulin G; NiV_{B}, Nipah virus Bangladesh strain; rVSV, recombinant vesicular stomatitis virus.

FIGS. 5A and 5B show the study designs for experimental challenge studies in AGMs. Experimental design for (A) Study 1, in which AGMs were vaccinated seven days prior to challenge with NiV_{B}, and (B) Study 2, in which AGMs were vaccinated three days prior to challenge. The red triangle indicates vaccination with the non-specific rVSV-ΔG-EBOV 76 vector, while the blue triangle denotes vaccination with the rVSV-ΔG-NiV_{B} G vector developed for these studies. Study days relative to challenge with 5×10⁵ PFU of NiV_{B} are shown below the horizontal black line, with days of blood collection indicated with arrows. AGM, African green monkey; EBOV, Zaire ebolavirus; NiV_{B}, Nipah virus Bangladesh strain; PFU, plaque-forming units; rVSV, recombinant vesicular stomatitis virus.

FIG. 6: Respiratory rates following challenge of AGMs with NiV_{B}. Line graph of respiratory rates in breaths per minute tracked daily throughout Study 1 for animals vaccinated with rVSV-ΔG-NiV_{B} G (blue symbols) or rVSV-ΔG-EBOV 76 (red symbols) seven days prior to challenge with NiV_{B}. Animal IDs are shown in the legend to the right of the graph. AGM, African green monkey; EBOV, Zaire ebolavirus; NiV_{B}, Nipah virus Bangladesh strain; rVSV, recombinant vesicular stomatitis virus.

FIGS. 7A to 7D show the viremia in vaccinated AGMs after challenge with NiV_{B}. Live virus isolated from EDTA plasma by plaque assay (panels A and C) or viral RNA detected in whole blood by qRT-PCR specific for NiV_{B} (panels B and D) for AGMs challenged with NiV_{B} seven days after vaccination (panels A and B) or three days after vaccination (panels C and D). Groups vaccinated with rVSV-ΔG-EBOV 76 are represented by red circles, and groups vaccinated with rVSV-ΔG-NiV_{B} G are represented by blue circles. Horizontal lines represent the mean of the values for all members of the group at each timepoint, and error bars represent the SEM. The LOD for the plaque assays is 25 PFU; qRT-PCR values are reported as 1 GEq/mL if they were below the LOD. AGM, African green monkey; EBOV, Zaire ebolavirus; EDTA, ethylenediaminetetraacetic acid; GEq, genome equivalent; LOD, limit of detection; mL, milliliter; NiV_{B}, Nipah virus Bangladesh strain; PFU, plaque-forming units; qRT-PCR, quantitative reverse transcriptase-polymerase chain reaction; SEM, standard error of the mean; rVSV, recombinant vesicular stomatitis virus.

FIGS. 8A and 8B show PRNT₅₀ results for AGMs vaccinated prior to challenge with NiV_{B}. PRNT₅₀ values from EDTA plasma, reported as reciprocal dilutions at which plaque counts were reduced by 50% compared to control wells, for animals vaccinated with rVSV-ΔG-NiV_{B} G (blue symbols) or rVSV-ΔG-EBOV 76 (red symbols) (A) seven days or (B) three days prior to challenge with NiV_{B}. Animal IDs are shown in the legend to the right of the graph. AGM, African green monkey; EBOV, Zaire ebolavirus; EDTA, ethylenediaminetetraacetic acid; NiV_{B}, Nipah virus Bangladesh strain; PRNT, plaque reduction neutralization test; rVSV, recombinant vesicular stomatitis virus.

FIG. 9: Vector and experimental design for the vaccination and challenge of AGMs. Schematic of the RNA genomes of rVSV-WT and rVSV-ΔG-NiV_{B} G. The NiV_{B} G gene (green box) was cloned into the native VSV G gene site (yellow box) in a plasmid containing the entire rVSV genome and recovered in VSV G-complemented (pC-VSV-G) baby hamster kidney cells. Intergenic and 3′ or 5′ untranslated genomic regions are indicated by black lines. (b) Seventeen AGMs were randomized into four groups: prime only (N=6), prime+boost (N=5), vector control prime (N=3), and vector control prime+boost (N=3) groups. Each group received a 1×10⁷ PFU i.m. dose of rVSV-ΔG-NiV_{B} G vaccine or a non-specific rVSV vector control expressing the Ebola virus glycoprotein (rVSV-ΔG-EBOV-GP). The prime+boost and vector control prime+boost groups received an additional dose at 56 days post vaccination. Blood samples were collected monthly at days 0, 10, 28, 56, 84, 112, 139, 164, 195, 221, 259, 294, 329, and 369 (0). AGMs were subsequently challenged one year later with a 5×10³ PFU of NiV_{B} i.n. dose delivered by MAD. Post-exposure blood samples were collected at 4, 7, 10, 14, 21, 28, terminally, and/or 35 days. Blue pins indicate vaccination phase sampling timepoints, whereas red lines denote challenge phase sampling timepoints. Abbreviations: RNA, ribonucleic acid; rVSV, recombinant vesicular stomatitis virus; WT, wild type; NiV_{B}, Nipah virus Bangladesh strain; N, nucleoprotein; P, phosphoprotein; M, matrix protein; G, glycoprotein. AGM, African green monkey; EBOV, Ebola virus; PFU, plaque-forming units; i.m., intramuscular; intranasal, i.n.; MAD, mucosal atomization device.

FIGS. 10A to 10C. Survival and health of vaccinated AGMs exposed to NiV_{B}. (FIG. 10A) Kaplan-Meier survival curves of vaccinated AGMs exposed to NiV_{B} for prime only (N=6; red lines), prime+boost (N=5; blue lines), vector control prime (N=3; dark gray lines), and vector control prime+boost (N=3; light gray lines) groups. A statistically significant association (** p-value <0.0021) was found between prime and vector control prime, and prime+boost and vector control prime+boost groups. (FIG. 10B) Clinical scores of individual AGMs vaccinated with rVSV-ΔG-NiV_{B} G or a non-specific rVSV vector control and challenged one year later with NiV_{B}. (FIG. 10C) Respiration rates represent the percentage above or below baseline pre-vaccination baseline values (beats per minute) of individual AGM subjects for each group challenged with NiV_{B}. Abbreviations: AGM, African green monkey; rVSV, recombinant vesicular stomatitis virus; NiV_{B}, Nipah virus Bangladesh strain; DPI, days post infection.

FIGS. 11A to 11D shows viral loads of immunized AGMs after challenge with NiV_{B}. Detection of NiV_{B} viral loads in EDTA plasma by plaque assay (FIG. 11A), whole blood by RT-qPCR (FIG. 11B), or (FIG. 11C, 11D) tissues by RT-qPCR for prime only (rVSV-ΔG-NiV_{B}G N=6; red bars), prime+boost (rVSV-ΔG-NiV_{B}G N=5; blue bars), vector control prime (rVSV-ΔG-EBOV-GP N=3; dark gray bars), and vector control prime+boost (rVSV-ΔG-EBOV-GP N=3; light gray bars) groups. Bars represent the mean value for all members of the group at each timepoint, and error bars represent the SEM. LOD for plaque assays is 25 PFU; LOD for RT-qPCR is 1000 copies/mL. Abbreviations: AGM, African green monkey; NiV_{B}, Nipah virus Bangladesh strain; RT-qPCR, quantitative reverse transcriptase-polymerase chain reaction; EBOV, Ebola virus; EDTA, ethylenediaminetetraacetic acid; LOD, limit of detection; mL, milliliter; PFU, plaque-forming units; SEM, standard error of the mean; rVSV, recombinant vesicular stomatitis virus. Open circles represent average values from duplicates for individual subjects. Two-way ANOVA with Tukey's multiple comparisons test. p-value <0.0332 (*), 0.0021 (**), 0.0002 (***), 0.0001 (****).

FIGS. 12A to 12T shows pathology of vaccinated and control NiV_{B}-infected AGMs. Representative photomicrographs of immunohistochemistry (IHC) for anti-NiV antigen (brown) in lung (FIGS. 12B, 12D), spleen (12F, 12H), liver (12J, 12L), kidney (12N, 12P) and brain (12R, 12T). Hematoxylin and Eosin (H&E) staining of the lung (12A, 12C), spleen (12E, 12G), liver (12I, 12K), kidney (12M, 12O) and brain (12Q, 12S). All photomicrographs taken at 20× magnification. Positive Control VC-P-3 (12A, 12B, 12E, 12F, 12I, 12J, 12M, 12N) and VC-P-2 (12Q, 12R). Loss of normal pulmonary alveolar architecture with inflamed and necrotic alveolar septa and flooding of alveolar spaces with fibrin, edema, hemorrhage (12A) IHC positive endothelium and mononuclear cells within the alveolar septa and alveolar macrophages (12B). Loss of splenic germinal center architecture with lymphocytosis, syncytial cell formation, and hemorrhage (12E). IHC positive mononuclear cells concentrated in the white pulp and scattered within the red pulp (12F). Sinusoidal leukocytosis (12I). IHC positivity of sinusoidal lining cells and Kupffer cells (black arrows) (12J), renal glomerular congestion (12M), segmental IHC positive glomerular endothelium and mononuclear cells (black arrow) (12N), diffuse gliosis of the brainstem (12Q), IHC positive neuronal cells of the brainstem (black arrow) (12R). No appreciable immunolabeling or lesions noted in the lung, spleen, liver, kidney, or brain of representative rVSV-ΔG-NiV_{B} G surviving AGM P-3 (12C, 12D, 12G, 12H, 12K, 12L, 12O, 12P, 12S, 12T).

FIGS. 13A to 13D show humoral responses in vaccinated AGMs. AGM serum samples were tested for circulating NiV G-specific IgG (FIG. 13A) and IgM (FIG. 13B) by indirect ELISA. The average reciprocal dilution titer for each group±the SEM at each timepoint is shown. (FIG. 13C) The average anti-NiV neutralizing antibody titer for each group±the SEM at each timepoint. PRNT₅₀ values represent the reciprocal dilution at which plaque counts were reduced by 50% compared to control wells. Each group is denoted by line color: prime only (rVSV-ΔG-NiV_{B}G N=6; red), prime+boost (rVSV-ΔG-NiV_{B}G N=5; blue), vector control prime (rVSV-ΔG-EBOV-GP N=3; dark gray), and vector control prime+boost (rVSV-ΔG-EBOV-GP N=3; light gray). (FIG. 13D) Correlation plots for respiration rates versus IgG, IgM, and neutralizing antibody levels. Abbreviations: vacc, vaccination; DPI, days post infection; NiV_{B}, Nipah virus Bangladesh strain; G, NiV_{B} glycoprotein; EBOV, Ebola virus; PRNT, plaque reduction neutralization test; rVSV, recombinant vesicular stomatitis virus; immunoglobulin M, IgM; immunoglobulin G, IgG. Two-way ANOVA with Tukey's multiple comparisons test. p-value <0.0332 (*), 0.0021 (**), 0.0002 (***), 0.0001 (****). A Pearson test was used to determine correlations.

FIGS. 14A to 14D shows the cellular responses in immunized AGMs. (14A) NiV G-specific IFNγ+ SFUs in PBMCs from vaccinated AGMs for each group. Values were calculated by subtracting the number of average spots from unstimulated duplicate wells from its respective stimulated counterpart at the corresponding DPI. (14B) CD4+ and (14C) CD8+ T cell counts in vaccinated AGM PBMCs at each timepoint. Each group is denoted by the following: prime only (rVSV-ΔG-NiV_{B}G N=6; red bars), prime+boost (rVSV-ΔG-NiV_{B}G N=5; blue bars), vector control prime (rVSV-ΔG-EBOV-GP N=3; dark gray bars), and vector control prime+boost (rVSV-ΔG-EBOV-GP N=3; light gray bars). Bars represent the mean value for all members of the group at each timepoint, and error bars represent the SEM. Open circles represent the average value of duplicates from individual subjects. (13D) Pie graphs depicting NiV G-specific CD4+ (top row) and CD8+ T cell (bottom row) cytokine profiles in PBMCs from each respective AGM group. The arcs denote the total percentage of degranulating (red), IFNγ+ (yellow), IL-2+ (green), and TNFα+ (teal) T cells. Each slice represents a specific combination of these markers. The vector control groups were combined for this analysis. Abbreviations: PBMC, peripheral blood mononuclear cells; SFU, spot-forming unit; AGM, African green monkey; vacc, vaccination; DPI, days post infection; NiV_{B}, Nipah virus Bangladesh strain; G, NiV_{B} glycoprotein; EBOV, Ebola virus; PRNT, plaque reduction neutralization test; rVSV, recombinant vesicular stomatitis virus.

FIGS. 15A to 15F shows the transcriptional responses in AGMs after challenge with NiV_{B}.

(15A) PCA based on DPI (0, 4, 7, 10/terminal timepoints) and each group: prime only (rVSV-ΔG-NiV_{B}G N=6; yellow), prime+boost (rVSV-ΔG-NiV_{B}G N=5; maroon), vector control prime (rVSV-ΔG-EBOV-GP N=3; purple), and vector control prime+boost (rVSV-ΔG-EBOV-GP N=3; lavender). (14B) overall expression changes for each group at late disease (orange denotes upregulated transcripts; blue denotes downregulated transcripts; black denotes no expression change). Heatmaps depicting the topmost downregulated (15C) and upregulated (15D) transcripts in specifically versus non-specifically vaccinated subjects at late disease (false discovery rate (FDR)-adjusted P values <0.05). A comparison of prime versus boosted subjects was also performed. Dots indicate transcripts mapping to interferon signaling (brown) and adaptive immunity (green) nSolver gene sets. For the heatmaps, red denotes upregulated transcripts, blue denotes downregulated transcripts, and white denotes no expression change. (15E) Trend plot depicting overall n-Solver derived cell-type quantities in control and vaccinated (fatal or survivor) cohorts. (15F) Pathway enrichment of differentially expressed transcripts in specifically vaccinated subjects at late disease. Displayed are the mean −log 10 (p-values). Horizontal lines within each plot indicate adjusted P value thresholds. Targets highlighted in blue indicate false discovery rate (FDR)-adjusted P values <0.10. A Benjamini-Hochberg test was employed to derive FDR-adjusted P values. Abbreviations: PCA, principal component analysis; DPI, days post infection; PC1, principal component 1; PC2, principal component 2; BH, Benjamini-Hochberg.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Nipah virus” refers to a zoonotic virus that can spread between animals and humans. Fruit bats (also referred to as flying foxes) are an animal reservoir for NiV in nature. Nipah virus is also known to cause illness in pigs and humans. Infection with NiV is associated with encephalitis (swelling of the brain) and can cause mild to severe illness and even death. Nipah virus infection can be prevented by avoiding exposure to sick pigs and bats. Nipah virus can also be contracted from drinking raw date palm sap contaminated by an infected bat.

As used herein, the terms “nucleic acid”, “nucleotide”, and “polynucleotide” are used interchangeably and refer to RNA, DNA, cDNA, or cRNA and derivatives thereof, such as those containing modified backbones. It should be appreciated that the invention provides polynucleotides comprising sequences complementary to those described herein. The “polynucleotide” contemplated in the present invention includes both the forward strand (5′ to 3′) and reverse complementary strand (3′ to 5′). Polynucleotides according to the invention can be prepared in different ways (e.g., chemical synthesis, gene cloning, etc.) and can take various forms (linear or branched, single or double stranded, or a hybrid thereof, primers, probes, etc.).

As used herein, the terms “genomic DNA” or “genome” are used interchangeably and refers to the genetic information of a host organism. The genomic DNA comprises the DNA of the nucleus (also referred to as chromosomal DNA) and cellular organelles (e.g., mitochondria). The genomic DNA or genome contemplated in the present invention also refers to the RNA of a virus. The RNA may be a positive strand or a negative strand RNA. The term “genomic DNA” includes the genomic DNA containing sequences complementary to those described herein. The term “genomic DNA” also refers to messenger RNA (mRNA), complementary DNA (cDNA), and complementary RNA (cRNA).

As used herein, the term “gene” refers to any segment of polynucleotide associated with a biological function. Thus, genes or polynucleotides include introns and exons as in genomic sequence, or just the coding sequences as in cDNAs, such as an open reading frame (ORF), starting from the start codon (methionine codon) and ending with a termination signal (stop codon). Genes and polynucleotides can also include regions that regulate their expression, such as transcription initiation, translation and transcription termination. Thus, also included are promoters and ribosome binding regions (in general these regulatory elements lie approximately between 60 and 250 nucleotides upstream of the start codon of the coding sequence or gene), transcription terminators (in general the terminator is located within approximately 50 nucleotides downstream of the stop codon of the coding sequence or gene). Gene or polynucleotide also refers to a nucleic acid fragment that expresses mRNA or functional RNA, or encodes a specific protein, and which includes regulatory sequences.

As used herein, the term “heterologous DNA” refers to the DNA derived from a different organism, such as a different cell type or a different species from the recipient. The term also refers to a DNA or fragment thereof on the same genome of the host DNA wherein the heterologous DNA is inserted into a region of the genome which is different from its original location.

As used herein, the terms “antigen” or “immunogen” refer to a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, or any combination thereof. The immunogen or antigen may comprise a toxin or antitoxin.

As used herein, the terms “immunogenic protein or peptide” or “vaccine” refers to polypeptide(s) that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. An immunogenic protein or polypeptide, or vaccine, includes the full-length sequence of the protein, analogs thereof, or immunogenic fragments thereof. An “immunogenic fragment” refers to a fragment of a protein that includes one or more epitopes and that elicits an immunological response. Such fragments can be identified using any number of epitope mapping techniques, well known in the art.

As used herein, the terms “conservative variation” refers to the replacement of an amino acid residue by another residue with the same basic structure (polar, non-polar, charged, etc.), or the replacement of a nucleotide in a nucleic acid sequence such that the encoded amino acid residue does not change or is a similar residue. In this regard, a substitution with a similar residue will generally be conservative in nature, i.e., those substitutions that take place within a family of amino acids. For example, amino acids are generally divided into four families: (1) acidic-aspartate and glutamate; (2) basic-lysine, arginine, histidine; (3) non-polar-alanine, valine, leucine, isoleucine, proline, phenylalanine, methionine, tryptophan; and (4) uncharged polar-glycine, asparagine, glutamine, cysteine, serine, threonine, tyrosine. Phenylalanine, tryptophan, and tyrosine are sometimes classified as aromatic amino acids. Examples of conservative variations include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like; or a similar conservative replacement of an amino acid with a structurally related amino acid that will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide. All of the polypeptides produced with these modifications are included herein. The term “conservative variation” also includes the use of a substituted amino acid in place of an unsubstituted parent amino acid provided that antibodies or T cell immunity raised to the substituted polypeptide also immunoreact with the unsubstituted polypeptide.

As used herein, the term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

As used herein, the term “immunological response” refers to a cellular and/or antibody-mediated immune response in the host to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the terms “recombinant” and “genetically modified” are used interchangeably and refer to any modification, alteration or engineering of a polynucleotide or protein in its native form or structure, or any modification, alteration or engineering of a polynucleotide or protein in its native environment or surrounding. The modification, alteration or engineering of a polynucleotide or protein may include, but is not limited to, deletion of one or more nucleotides or amino acids, deletion of an entire gene, codon-optimization of a gene, conservative substitution of amino acids, insertion of one or more heterologous polynucleotides.

As used herein, the terms “polyvalent vaccine or composition”, “combination or combo vaccine or composition” and “multivalent vaccine or composition” are used interchangeably to refer to a composition or vaccine containing more than one composition or immunogen. The polyvalent vaccine or composition may contain two, three, four or more compositions or vaccines. The polyvalent vaccine or composition may comprise recombinant viral vectors, active or attenuated or killed wild-type viruses, or a mixture of recombinant viral vectors and wild-type viruses in active or attenuated or killed forms.

As used herein, the term “homologs” includes orthologs, analogs and paralogs. The term “analogs” refers to two polynucleotides or polypeptides that have the same or similar function, but that have evolved separately in unrelated organisms. The term “orthologs” refers to two polynucleotides or polypeptides from different species, but that have evolved from a common ancestral gene by speciation. Normally, orthologs encode polypeptides having the same or similar functions. The term “paralogs” refers to two polynucleotides or polypeptides that are related by duplication within a genome. Paralogs usually have different functions, but these functions may be related. Analogs, orthologs, and paralogs of a wild-type polypeptide can differ from the wild-type polypeptide by post-translational modifications, by amino acid sequence differences, or by both. In particular, homologs of the invention will generally exhibit at least 80-85%, 85-90%, 90-95%, or 95%, 96%, 97%, 98%, 99% sequence identity, with all or part of the polynucleotide or polypeptide sequences of antigens described above, and will exhibit a similar function.

In one embodiment, the present invention provides a recombinant VSV (rVSV) viral vector comprising one or more heterologous polynucleotides coding for and expressing the NiVb-G antigen or polypeptide. In one aspect of the embodiment, the NiVb-G antigen or polypeptide has at least 70%, 75%, 80%, 85%, 90%, 95%, 96%, 97%, 98%, 99% or 100% sequence identity to a polypeptide having the sequence as set forth in SEQ ID NO: 6, or a conservative variant, an allelic variant, a homolog or an immunogenic fragment comprising at least eight or at least ten consecutive amino acids of one of these polypeptides, or a combination of these polypeptides in a recombinant VSV (rVSV) viral vector.

In another aspect of the invention, the polynucleotide encoding an NiVb-G antigen or polypeptide having at least 70%, 75%, 80%, 85%, 90%, 95%, 96%, 97%, 98%, 99% or 100% sequence identity to a polypeptide having the sequence as set forth in SEQ ID NO: 6 recombinant by a VSV (rVSV) viral vector. In yet another aspect of the embodiment, the heterologous polynucleotide has at least 70%, 75%, 80%, 85%, 90%, 95%, 96%, 97%, 98%, 99% or 100% sequence identity to a polynucleotide having the sequence as set forth in SEQ ID NO: 2.

As used herein, the term “identity” with respect to sequences can refer to, for example, the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman). The sequence identity or sequence similarity of two amino acid sequences, or the sequence identity between two nucleotide sequences can be determined using Vector NTI software package (Invitrogen, 1600 Faraday Ave., Carlsbad, Calif.). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

The polynucleotides of the present invention include sequences that are degenerate as a result of the genetic code, e.g., optimized codon usage for a specific host.

As used herein, the term “optimized” refers to a polynucleotide that is genetically engineered to increase its expression in a given species. To provide optimized polynucleotides coding for AIV-HA polypeptides, the DNA sequence of the NiVb-G antigen or polypeptide can be modified to 1) comprise codons preferred by highly expressed genes in a particular species; 2) comprise an A+T or G+C content in nucleotide base composition to that substantially found in said species; 3) form an initiation sequence of said species; or 4) eliminate sequences that cause destabilization, inappropriate polyadenylation, degradation and termination of RNA, or that form secondary structure hairpins or RNA splice sites. Increased expression of the NiVb-G antigen or polypeptide can be achieved by utilizing the distribution frequency of codon usage in eukaryotes and prokaryotes, or in a particular species. As used herein, the term “frequency of preferred codon usage” refers to the preference exhibited by a specific host cell in usage of nucleotide codons to specify a given amino acid. There are 20 natural amino acids, most of which are specified by more than one codon. Therefore, all degenerate nucleotide sequences are included in the present invention as long as the amino acid sequence of NiVb-G antigen or polypeptide encoded by the nucleotide sequence is functionally unchanged.

Nipah viruses. In 1998, Nipah virus (NiV) emerged and was shown to be a previously unknown paramyxovirus, now classified along with Hendra virus (HeV) and Cedar virus within the Henipavirus genus. NiV causes febrile encephalitis and severe respiratory disease in humans, with a case-fatality rate (CFR) as high as 100% in some outbreaks. Outbreaks of NiV have occurred almost annually in Bangladesh and India from 2001 to 2020, and a 2018 outbreak in India caused 17 deaths out of 19 cases. Genetic analysis has identified at least two strains of NiV responsible for outbreaks in different geographical areas. The Malaysia strain (NiV_{M}) caused the initial outbreak of NiV from 1998-1999 in Malaysia and Singapore in which over 270 people were infected with a CFR of about 40%, and an additional 2014 outbreak in the Philippines had a CFR of approximately 52%. The Bangladesh strain (NiV_{B}), however, has caused outbreaks of NiV_{B} associated with person-to-person transmission, including in healthcare settings, and with higher CFRs, averaging about 75%. Importantly, the inventors have developed nonhuman primate (NHP) models for both NiV_{M}¹ and NiV_{B}² and recently showed that NiV_{B} is more pathogenic in African green monkeys (AGMs) than NiV_{M} under identical experimental conditions². The inventors also showed that treatments that protect AGMs against NiV_{M} were not as effective against NiV_{B}², demonstrating the importance of any medical countermeasure to protect against the ongoing public health threat of the more virulent NiV_{B}.

NiV has a negative-sense, single-stranded RNA genome comprising six genes: N, P, M, F, G and L. The F gene encodes the fusion protein, which allows for fusion of the viral and host cell membranes during viral entry. The G gene encodes the glycoprotein, which is the viral attachment protein that recognizes host ephrin-B2 and ephrin-B3 receptors expressed on endothelial cells and neurons that are highly conserved among many mammalian species. Vaccine studies in pre-clinical animal models of NiV disease have focused primarily on the F and G proteins as antigens.

Currently, there are no vaccines licensed for the prevention of NiV disease. To date, there have been at least seven experimental preventive candidate vaccines against henipaviruses evaluated in NiV_{M} pre-clinical animal models. Experimental vaccines encoding NiV_{M} F or NiV_{M} G and vectored with vaccinia, canarypox, adeno-associated, or measles viruses have been tested, as have NiV virus-like particle (VLP) and recombinant subunit (HeV G) vaccines. While the majority of these platforms demonstrated a robust immune response in laboratory animals, most of these vaccines require multiple injections to confer protective efficacy. However, in the setting of pathogens such as NiV, which are indigenous to regions of Asia and are also potential agents of bioterrorism, a single-injection vaccine is preferable. Multiple-dose vaccines are costly and impractical for the prevention of natural infections in developing countries due to logistics and compliance concerns. In the case of a deliberate release of NiV, there would be little time for deployment of a vaccine that requires multiple injections over an extended period of time. Thus, for most practical applications, a vaccine against NiV necessitates a single immunization. Moreover, all the pre-clinical animal studies mentioned above assessed efficacy against the less pathogenic NiV_{M} strain, rather than the more pathogenic NiV_{B} strain. While the antigenicity of these vaccines should not be a concern considering that the HeV G protein can protect against NiV_{M} infection, data using the NiV_{B} AGM model highlights the importance of testing with the more highly pathogenic strain. As noted above, NiV_{B} infection in AGMs is more pathogenic when compared to NiV_{M} infection². Importantly, this difference resulted in significantly reduced efficacy of antibody therapy. Specifically, the human monoclonal antibody m102.4, which had previously been shown to completely protect AGMs against lethal NiV_{M} disease when treatment was delayed until day 5 after virus exposure, failed to provide any protection when AGMs were challenged with NiV_{B} and treated beginning at day 5 after virus challenge². Therefore, the current vaccines against NiV need to be tested against the more pathogenic NiV_{B} infection in the robust AGM model.

The present invention includes a recombinant vesicular stomatitis virus (rVSV)-based NiV vaccine. VSV is a non-segmented, negative-sense RNA virus in the family Rhabdoviridae. It is primarily an animal pathogen and is not known to cause severe disease in humans. VSV is the prototypic rhabdovirus and possesses a number of characteristics that are important for a vaccine vector: replication in almost all known mammalian cell lines, growth to very high titers, and a strong induction of innate and adaptive (humoral as well as cellular) immune responses. In addition, there are very low levels of pre-existing immunity to VSV in the general population, with the neutralizing immune response primarily directed against the VSV glycoprotein (VSV G), a viral protein that is not expressed in rVSV vaccine vectors (referred to as delta G or AG vaccine vectors).

Several groups have developed rVSV-vectored NiV vaccines. A replication-competent rVSV-NiV G vaccine was highly efficacious in hamsters³ and provided protection when given only one day before exposure to NiV_{M}⁴. This vaccine also protected AGMs from NiV_{M} disease one month after a single intramuscular (i.m.) administration⁵. However, these vaccines relied on inclusion of the Ebola virus (EBOV) glycoprotein for replication and has not yet been tested against NiV_{B}. Single-cycle rVSVs, which undergo just one cycle of replication after vaccination due to expression of just one of the two NiV surface proteins, have also been developed against NiV. One such vaccine has shown strong immunogenicity in mice vaccinated with rVSVs expressing either NiV_{M} F or the NiV_{M} G, as high neutralizing antibody titers were generated⁶. These vaccine vectors were shown to provide homologous protection in the hamster model of NiV_{M} infection⁷.

The present inventors developed a single-cycle rVSV vaccine vectors expressing either the NiV_{B} F or NiV_{B} G proteins. These vaccines were evaluated 28 days after a single-dose vaccination in the NiV_{M} ferret model and were shown to completely protect ferrets from lethal challenge⁸. More recently, these constructs demonstrated complete protection of AGMs when given as a single i.m. dose 28 days prior to challenge with NiV_{B}⁹, constituting the first reported protection of AGMs from NiV_{B} using a single-dose rVSV vaccine. The inventors developed the novel rVSV-NiV_{B} G vaccine and found that animals vaccinated with this construct demonstrated more consistent neutralizing antibody titers and fewer clinical pathology changes than those vaccinated with constructs expressing NiV_{B} F alone or both NiV_{B} F and G⁹. A prior version of a vaccine^{8,9} encoded green fluorescent protein (GFP) along with NiV_{B} G protein.

### Example 1. Single-Dose, Single-Cycle, VSV-Vectored Vaccine Protects African Green Monkeys when Given Shortly Prior to Nipah Virus Bangladesh Challenge

NiV_{B} causes small, sporadic outbreaks of NiV disease with high case-fatality rates in Bangladesh and India. During an outbreak in the state of Kerala, India, in 2018, NiV_{B} spread to family and hospital caregivers through close contact and led to 23 identified cases and 21 deaths¹. Most recently, six cases were identified in Bangladesh in 2020 that led to a reported four deaths². Person-to-person transmission is a common feature of outbreaks of NiV_{B}^{3,4}. A human monoclonal antibody is currently available for compassionate use in symptomatic patients or after laboratory exposures, but no licensed vaccine is available for prevention of infection⁵⁻⁷. A vaccine that works rapidly is urgently needed to prevent spread during outbreaks of this highly lethal pathogen.

Recombinant vesicular stomatitis virus (rVSV) has been used as a vaccine vector to deliver NiV vaccines for over a decade. These vectors usually replace the VSV G surface protein with a glycoprotein from a pathogen of interest; the resulting constructs are called rVSV-ΔG vectors and are pseudotyped with the antigen of interest. Since the NiV genome encodes two surface proteins, F and G, which are both required for viral entry, rVSV-ΔG vectors encoding only one of these antigens are replication-incompetent and must be complemented with the other surface protein or with the missing VSV G protein for amplification of viral stocks. Co-transfection of rVSV-ΔG genomes containing either NiV F or NiV G into the same cells allows them to complement each other and produces single-cycle rVSV-ΔG viruses expressing both proteins on their surface⁸. These replication-incompetent vectors encoding either NiV_{M} F or G protected Syrian hamsters from challenge with NiV_{M} when given 32 days prior to challenge⁹. Similarly, single-cycle rVSV-ΔG vectors expressing NiV_{B} F, G, or both along with green fluorescent protein (GFP) protected ferrets from challenge with NiV_{M} when given 28 days prior to challenge and induced strong neutralizing antibody responses¹⁰. The rVSV-ΔG-NiV_{B} G-GFP construct from that study was given to AGMs 28 days prior to challenge and protected them from otherwise lethal doses of NiV_{M} or NiV_{B}¹¹.

Another approach to combat the replication incompetence of rVSV-ΔG-NiV vectors is to co-express a NiV surface protein with another viral protein capable of viral entry on its own, such as Zaire ebolavirus (EBOV) glycoprotein (GP), within the rVSV-ΔG backbone. Such vectors containing NiV_{M} F or G protected 100% of Syrian hamsters as well as AGMs when given 28 days (hamsters) or 29 days (AGMs) prior to challenge with NiV_{M}, and protection could be passively transferred to naïve hamsters^{12,13}. These constructs also protected hamsters when given very close to challenge, although the influence of innate immune responses to either the vector or the EBOV antigen could not be distinguished from NiV-specific, adaptive immune responses to the vaccines¹⁴.

Clearly, rVSV-ΔG vaccine constructs are highly effective at protecting ferrets, Syrian hamsters, and AGMs from lethal challenge with either NiV_{M} or NiV_{B} in a single dose, whether replication-competent or -incompetent. A study investigating rabies virus-vectored vaccines for NiV used an rVSV vector as a comparator and found that the immune responses elicited by the rVSV vector were more robust than those induced by the rabies-vectored constructs¹⁵. Moreover, a previous study with rVSV constructs encoding human immunodeficiency virus proteins demonstrated that replication-incompetent rVSVs induce immune responses of equivalent strength and efficacy to those induced by replication-competent rVSVs¹⁶. Given that NiV_{B} appears to be more virulent than NiV_{M} in the AGM model and has a shorter therapeutic window for treatment with m102.4 monoclonal antibody^{17,18}, and given that the inventors saw the most consistent and robust neutralizing antibody responses to the vaccine encoding the NiV_{B} G antigen¹¹, the inventors chose to move forward with an rVSV-ΔG construct encoding NiV_{B} G. Based on these previous data, the inventors developed an rVSV-ΔG-NiV_{B} G vaccine construct that is replication-incompetent and yet protects AGMs from NiV_{B} challenge in a single dose when delivered shortly prior to challenge. Such a vaccine would have the necessary rapid efficacy and safety profile for deployment in an outbreak scenario to prevent further spread of NiV.

In this example, the inventors described the rVSV-ΔG-NiV_{B} G vaccine developed without GFP and rescued using established reverse genetics system. AGMs were vaccinated either seven or three days prior to challenge with NiV_{B} by the i.n./intratracheal (i.t.) route. The rVSV-ΔG-NiV_{B} G vaccine protected 100% of vaccinated AGMs from lethal outcome when given seven days prior to challenge and 67% of vaccinated AGMs when given three days prior to challenge. Efforts to define a survivor phenotype and to evaluate humoral and cellular NiV-specific immune responses are underway and will be discussed.

Cell culture. BHK-21 clone WI-2 cells were a generous gift from Dr. Michael A. Whitt (currently at The University of Tennessee Health Science Center; Memphis, Tennessee, USA)¹⁹. BHK-21 clone WI-2 cells were maintained in high-glucose DMEM supplemented with 5% heat-inactivated FBS, 1% penicillin/streptomycin solution (10,000 units/mL penicillin and 10,000 μg/mL streptomycin), and 1% GlutaMAX™ Supplement. Vero 76 monkey kidney cells (ATCC CRL-1587) were maintained as instructed for ATCC CRL-1587.

Cloning to produce the full-length pVSV-ΔG-NiV_{B} G plasmid. The pVSV-ΔG-NiV_{B} G plasmid was made by Gibson assembly of fragments encoding the NiV_{B} G gene and the remainder of the pVSV-ΔG backbone²⁰. To generate vector material for Gibson assembly, a previously constructed pVSV-ΔG plasmid was digested with MluI-HF and AvrII restriction enzymes (New England Biolabs; Cat. Nos. R3198 and R0174, respectively) and purified by SDS-PAGE electroelution. Oligonucleotide PCR primers designed with large overhangs for Gibson assembly were ordered from IDT. Inserts were generated by using the primers to amplify the NiV_{B} G gene out of the previous pVSV-ΔG-NiV_{B} G-GFP construct by PCR using Platinum™ SuperFi™ DNA polymerase according to manufacturer instructions. The full-length pVSV-ΔG-NiV_{B} G construct, encoding rVSV-ΔG with NiV_{B} G in place of VSV G in the pBluescript (pBS) plasmid backbone, was assembled using the NEBuilder® HiFi DNA Assembly Cloning Kit according to manufacturer instructions. Presence of the NiV_{B} G gene was confirmed and cloning borders checked by Sanger sequencing and diagnostic restriction digest. A large culture of E. coli transformed with the construct was grown, and plasmid was purified by alkaline lysis cesium chloride plasmid prep as previously described¹⁹.

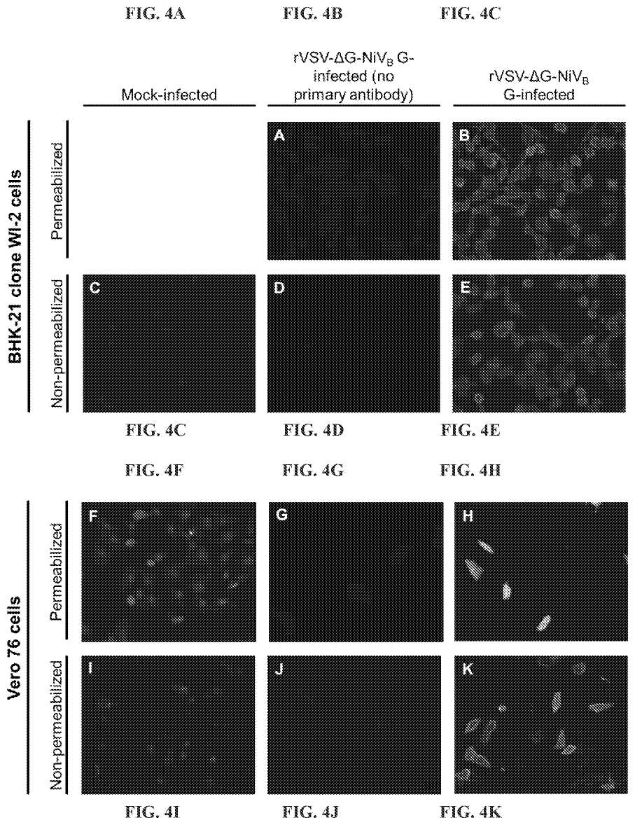

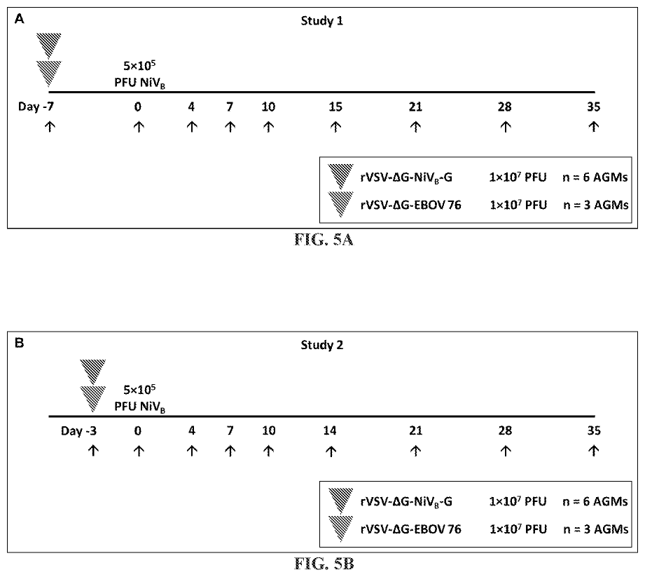

Recovery, amplification, and quality control validation of the rVSV-ΔG-NiV_{B} G vaccine. The rVSV-ΔG-NiV_{B} G vaccine construct was recovered using a protocol modified from Dr. Michael A. Whitt¹⁹. A schematic showing the process for recovery of the construct in BHK-21 clone WI-2 cells is shown in FIG. 4A. Cells were seeded in 6-well plates to be 70 to 80% confluent for infection and transfection the next day. First, the cells were infected with modified vaccinia virus expressing bacteriophage T7 polymerase (vTF7-3; ATCC; Cat. No. VR-2153) at an MOI of 5. Immediately afterward, cells were transfected with pBS-VSV G, pBS-VSV N, pBS-VSV P, pBS-VSV L, and the full-length pVSV-ΔG-NiV_{B} G plasmid (all under the control of the T7 promoter) in a ratio of 8:3:5:1:5 using TransfectACE reagent¹⁹ at a ratio of 3.5 μL per μg of plasmid DNA. Control wells were transfected with pVSV-ΔG-GFP or pVSV-ΔL-GFP and the helper plasmids. Plates were incubated at 37° C. and 5% CO₂ for four to five hours, at which point the growth medium was changed. Plates were returned to the incubator for 48 hours to allow production of infectious virions.

FIG. 3: Recovery of rVSV-ΔG-NiV_{B} G virions. Diagram of the process by which recovery of rVSV-ΔG-NiV_{B} G virions occurs in BHK-21 clone WI-2 cells. 1. Cells are infected with vTF7-3 to produce bacteriophage T7 polymerase. 2. Cells are transfected with the full-length pVSV-ΔG-NiV_{B} G plasmid and helper plasmids encoding VSV G, VSV N, VSV P, and VSV L (all under the control of the T7 promoter). 3. The helper plasmids are transcribed and translated to produce VSV N, P, and L protein; the full-length plasmid is transcribed to produce the RNA genome of rVSV-ΔG-NiV_{B} G. Together, they form the RNP complex. 4. The RNA genome is transcribed to produce mRNAs encoding VSV N, P, M, and L and NiV_{B} G. 5. The mRNAs are translated to produce VSV N, P, M and L and NiV_{B} G proteins. 6. The RNA genome and viral structural proteins are packaged into virions near the cell membrane. 7. Live virions bud out from the cell with NiV_{B} G protein on their surface. BHK, baby hamster kidney; mRNA, messenger ribonucleic acid; NiV_{B}, Nipah virus Bangladesh strain, RNA, ribonucleic acid; RNP, ribonucleoprotein; rVSV, recombinant vesicular stomatitis virus; VSV, vesicular stomatitis virus. Figure created using BioRender.com.

Because the rVSV-ΔG-NiV_{B} G vaccine encodes only one of the two NiV surface proteins, it requires supplementation with VSV G provided in trans for propagation in cells. BHK-21 clone WI-2 cells were seeded in 6-well plates to be 70 to 80% confluent for transfection the next day. Cells were transfected with 1 μg of pCAGGS-VSV G plasmid per well with 3 μL per well of Lipofectamine™ 2000 Transfection Reagent (Thermo Fisher Scientific; Cat. No. 11668019). Plates were incubated at 37° C. and 5% CO₂ for four hours, the growth medium was changed, and the plates were returned to the incubator overnight to allow expression of VSV G and formation of syncytia. These cells are hereafter called G-complemented (G*) BHK cells.

After the 48-hour incubation, supernatant was removed from the pVSV-ΔG-NiV_{B} G transfection wells and filtered through a 0.22-μm Millex-GS syringe filter (Millipore; Burlington, Massachusetts, USA; Cat. No. SLGSM33SS) to remove vTF7-3. The filtered supernatant was then used to infect the G* BHK cells by incubating 500 μL per well for one hour at 37° C. and 5% CO₂. After adsorption, 2 mLs per well of complete DMEM with 5% FBS was added to the cells. Plates were incubated at 37° C. and 5% CO₂ for 48 to 72 hours to allow VSV-specific CPE to develop. Once cells exhibited 40 to 100% VSV-specific CPE, supernatants were collected, clarified, aliquoted into 2-mL externally threaded screwcap tubes with O-rings, and frozen at −80° C.

Next, recovery supernatants were passaged on fresh G* BHK cells for plaque purification. Picked plaques were collected into 2-mL screwcap tubes containing DMEM with 5% FBS, incubated at 37° C. for 1 hour with intermittent vortexing, and then the medium was used to infect fresh G* BHK cells for passage 1 (p1). Positive p1 supernatants were collected when VSV-specific CPE was observed (about 24 hpi), clarified to remove cell debris, and used to infect 10-centimeter cell culture dishes of G* BHK cells at an MOI of 1 for p2. At 24 hpi, VSV-specific CPE was observed, and the supernatant was collected, clarified, and aliquoted into 2-mL screwcap tubes for storage of p2 G*-rVSV-ΔG-NiV_{B} G seed stocks at −80° C.

Viral titers were determined using a standard plaque assay by infecting duplicate wells of G* BHK cells in 6-well plates with 200 μL each of 10-fold serial dilutions of viral stocks. After one-hour adsorption, plates were overlaid with two mLs per well of medium containing final concentrations of 1×MEM, 5% FBS, and 0.8% SeaKem® ME agarose. At 24 hpi, plaques were stained with a solution of 5% neutral red and 5% FBS in PBS without calcium and magnesium, and plaques were visualized and counted four hours later.

Viral RNA was isolated from p2 seed stock in TRIzol™ LS Reagent using the Direct-zol RNA miniprep kit according to manufacturer instructions. The complete viral RNA genome was sequenced with the NextSeq 550 system with a depth of 130 million reads. DNA was extracted from p2 seed stock in TRIzol™ LS according to manufacturer instructions for Mycoplasma testing with the e-Myco™ plus Mycoplasma PCR Detection Kit (LiliF Diagnostics; Burlington, Massachusetts, USA; Cat. No. 25238). The p2 seed stock was subjected to endotoxin testing using Endosafe®-PTS™ Limulus Amebocyte Lysate cartridges (Charles River Laboratories; Wilmington, Massachusetts, USA; Cat. No. PTS2001).

### Immunofluorescence Assay (IFA)

BHK-21 clone WI-2 cells and Vero 76 cells were seeded in 6-well plates at a density of 2×10⁵ cells per well to be 50 to 70% confluent for infection the next day. Cells were infected with G*-rVSV-ΔG-NiV_{B} G at an MOI of 3 or mock-infected with growth media only. At eight hpi, cells were fixed with a solution of 4% paraformaldehyde (made from 16% stock; Electron Microscopy Sciences; Hatfield, Pennsylvania, USA; Cat. No. 15710) in water and then quenched overnight in PBS with 100 mM glycine (PBS-glycine; made from Dulbecco's PBS with calcium and magnesium, Thermo Fisher Gibco, Cat. No. 14040; and glycine powder, Amresco, Dallas, Texas, USA, Cat. No. 0167-5KG). Half of the wells for each cell line were permeabilized with 0.5% Triton™ X-100 (Sigma-Aldrich; Cat. No. T8787-100ML) in PBS-glycine, while the remaining wells were processed without permeabilization. All wells were blocked for one hour in 3% sterile bovine serum albumin (BSA; Thermo Fisher Gibco; Cat. No. 15260-037) in PBS (hereafter known as blocking buffer). Conditions receiving human m102.4 primary antibody were incubated with 50 μg/mL antibody in blocking buffer at 4° C. overnight⁶, while secondary-only infected wells were incubated in blocking buffer only at 4° C. overnight.

All wells were washed three times with blocking buffer and then incubated with secondary antibody solution comprising goat anti-human IgG conjugated to Alexa Fluor™ 488 (dilution 1:5,000; Thermo Fisher Invitrogen; Cat. No. A11013) in blocking buffer for one hour, protected from light. Wells were then washed three times with blocking buffer and stored under PBS-glycine for imaging on a Nikon Eclipse Ti inverted fluorescent microscope (Nikon; Minato City, Tokyo, Japan). Images were obtained using the fluorescein isothiocyanate (FITC) filter and were exposed for 300 milliseconds at 20% power. Magnification from eyepiece and objectives totaled 100×.

Animal Studies. Animal Handling and Procedures.

Protocols for animal studies were approved by UTMB's IACUC and complied with the Animal Welfare Act and the Guide for the Care and Use of Laboratory Animals, National Research Council. Animal studies were performed under BSL-4 biocontainment in the UTMB GNL, which is fully accredited by the Association for Assessment and Accreditation of Laboratory Animal Care International.

For each study, nine adult AGMs (three to eight kilograms in weight) were randomized to the rVSV-ΔG EBOV 76 control group or the rVSV-ΔG-NiV_{B} G vaccinated group (three females and three males in the vaccinated group and two of one sex and one of the other in the control group). Animals were anesthetized with ketamine according to body weight for each procedure and received 1×10⁷ PFU of the appropriate vaccine intramuscularly (i.m.) either seven (Study 1) or three (Study 2) days prior to inoculation with 5×10⁵ PFU of NiV_{B}, split equally between the i.n. and i.t. routes. On days 0, 4, 7, 10, 14 (Study 2) or 15 (Study 1), 21, 28, and 35 after challenge and at terminal endpoint, subjects were anesthetized and examined, body temperature and weight were measured, and blood was collected. Subjects were assessed for clinical signs and respiration speed and quality daily after challenge. Clinical observations were scored on a scale from 0 to 29 based on respiration, appetite, activity/appearance, and neurologic signs; animals scoring 9 or greater were humanely euthanized according to the animal protocol.

### Blood Collection and Processing

At each timepoint and at terminal endpoint, blood was collected into a 4-mL Vacutainer® serum separator tube (BD; Franklin Lakes, New Jersey, USA; Cat. No. 367812), a 3-mL Vacutainer® EDTA tube (BD; Cat. No. 367856), and a 4-mL Vacutainer® lithium heparin tube (BD; Cat. No. 367884) for each animal and processed immediately after collection. From the EDTA tube, 100 μL of whole blood was added to 600 μL of AVL Viral Lysis Buffer with carrier RNA (Qiagen; Hilden, Germany; Cat. No. 19073) and incubated for at least ten minutes to inactivate virus. Inactivated material was transferred to a 1.8-mL Nunc™ cryovial (Thermo Scientific; Cat. No. 363401) and removed from the BSL-4. RNA was extracted using the QIAamp Viral RNA Mini Kit (Qiagen; Cat. No. 52906) according to manufacturer instructions.

Another 150 μL of whole blood was removed from the EDTA tube and placed into a 0.5-mL screwcap tube for hematology analysis using the VetScan® HM5 Hematology Analyzer (Abaxis, Inc.; Union City, California, USA). Complete blood counts including numbers of white blood cells, lymphocytes, monocytes, eosinophils, basophils, red blood cells, and platelets and measures of hematocrit and total hemoglobin were obtained using a protocol optimized for nonhuman primates according to manufacturer instructions.

The serum separator tubes and EDTA tubes were spun in a tabletop centrifuge at 2500 revolutions per minute (RPM) for 10 minutes at 4° C. to separate serum and plasma from cellular material. Serum was transferred to a 2-mL screwcap tube, used for clinical chemistry analysis (see below), and stored at −80° C. EDTA plasma was transferred to a 2-mL screwcap tube and stored at −80° C. for later use in immunological assays.

Analysis of clinical chemistry was performed using serum, Piccolo® BioChemistry Panel Plus reagent discs (Abaxis, Inc.; Cat. No. 400-7182-1), and the Piccolo® Xpress chemistry analyzer (Abaxis, Inc.). The BioChemistry Panel Plus measures levels of alanine aminotransferase (ALT), albumin, alkaline phosphatase, amylase, aspartate aminotransferase (AST), blood urea nitrogen (BUN), C-reactive protein (CRP), calcium, creatinine (CRE), gamma glutamyltransferase, glucose, total protein, and uric acid in serum.

The lithium heparin tubes and cell pellets from the EDTA tubes (resuspended in HBSS) were used for isolation of peripheral blood mononuclear cells (PBMCs) for downstream immunological analysis. Briefly, the buffy coat was isolated by centrifugation using 12-mL sterile ACCUSPIN™ tubes (Sigma-Aldrich; Cat. No. A1805) containing Histopaque®-1077 reagent (Sigma-Aldrich; Cat. No. 1077-1). Cells were washed, treated with Ammonium-Chloride-Potassium Lysing Buffer (Thermo Fisher Gibco; Cat. No. A1049201) to remove red blood cells, and enumerated using a TC20 Automated Cell Counter (Bio-Rad Laboratories; Cat. No. 1450102). PBMCs were resuspended in freezing medium comprising 10% dimethyl sulfoxide (Fisher Scientific; Cat. No. BP231-100) in FBS and were dispensed evenly across five 1.8-mL cryovials and frozen in a Mr. Frosty™ controlled-rate freezing device (Thermo Scientific; Cat. No. 5100-0001) and stored at −80° C.

### Tissue Collection and Processing.

At terminal or study endpoint, the following tissues were collected from each AGM for histopathology analysis, virus enumeration by plaque assay, and RNA extraction: axial lymph node, inguinal lymph node, liver, spleen, kidney, adrenal gland, lung (right upper, right middle, right lower, left upper, left middle, and left lower lobes), brain (frontal lobe, brain stem, and cervical spinal cord), pancreas, urinary bladder, ovary or testis, uterus or prostate, nasal mucosa, conjunctiva, and eye.

For virus enumeration, tissue samples were stored at −80° C. for later processing. Tissues were homogenized in cell culture medium in 2-mL screwcap tubes containing 1.4-millimeter ceramic beads (Omni International; Kennesaw, Georgia, USA; Cat. No. 19-627) using the TissueLyser II (Qiagen; Cat. No. 85300) to create 10% homogenate. Samples were spun in a microcentrifuge at 4° C. to pellet beads and debris, and supernatants were transferred to new 2-mL screwcap tubes and stored at −80° C. until titration (see below).

For RNA extraction, approximately 100 mg of tissue was stored in 1 mL of RNAprotect reagent (Qiagen; Cat. No. 76106) for later processing. RNAprotect reagent was removed, and tissues were homogenized in 600 μL of RLT lysis buffer (Qiagen; Cat. No. 79216) in 2-mL screwcap tubes containing ceramic beads using the TissueLyser II. Samples were spun in a microcentrifuge to pellet beads and debris, and supernatants were transferred to 1.8-mL cryovials and removed from the BSL-4. RNA was extracted using the RNeasy Mini Kit (Qiagen; Cat. No. 74106) according to manufacturer instructions.

Determination of viral load by plaque assay and quantitative (q)RT-PCR. Isolated RNA from blood and tissues was subjected to qRT-PCR using primers and probes specific to NiV_{B} targeting the N gene and the N-P intergenic region. Inclusion of the intergenic region prevents detection of viral mRNA by the assay. The probe was ordered from Thermo Fisher Invitrogen and featured 6-carboxyfluorescein (6FAM) fluorescent reporter dye at the 5′ end and tetramethylrhodamine (TAMRA) quencher at the 3′ end, with a nucleotide sequence of 5′ CGT CAC ACA TCA GCT CTG AGA A 3′. NiV_{B} viral RNA was detected using the OneStep RT-PCR kit (Qiagen; Cat. No. 210215) and the CFX96 Touch Real-Time PCR Detection System (Bio-Rad Laboratories). CFX Manager™ software (Bio-Rad Laboratories) was used to evaluate threshold cycle values, and results are reported as genome equivalent (GEq), determined from a plasmid standard using Avogadro's number and the molecular weight of the NiV_{B} genome.

Viral titers from blood and tissues were determined using a standard plaque assay on Vero 76 cells. Briefly, duplicate wells of 6-well plates were inoculated with 200 μL each of 10-fold serial dilutions of each sample, adsorbed for one hour at 37° C. and 5% CO₂, and then overlaid with two mLs per well of medium containing final concentrations of 1×MEM, 5% FBS, and 0.8% SeaKem® ME agarose. At 48 hpi, plates were stained with a solution of 5% neutral red and 5% FBS in PBS without calcium and magnesium, and plaques were visualized and counted at 72 hpi.

### NiV_{B} Plaque Reduction Neutralization Tests (PRNTs)

PRNTs were performed using EDTA plasma saved from each timepoint to give an estimate of neutralizing antibodies present in the blood at timepoints following vaccination/challenge. Plasma samples were heat-inactivated and serially diluted two-fold. They were then incubated with about 100 PFU per sample of wild-type NiV_{B} for one hour at 37° C. and 5% CO₂. Following incubation, virus/plasma mixes were plated on duplicate wells of Vero 76 cells and quantified by plaque assay with neutral red staining as described in previous sections. The PRNT₅₀ for each sample is reported as the reciprocal dilution at which plaque counts are 50% lower than control wells containing virus but no plasma.

The rVSV-ΔG-NiV_{B} G Vaccine Grows to High Titers in Cell Culture and Expresses the NiV_{B} G Protein

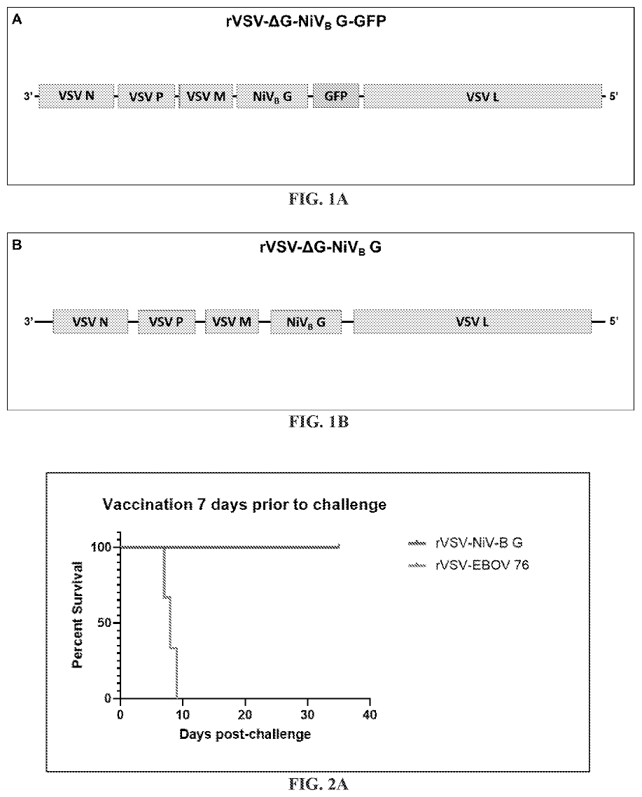

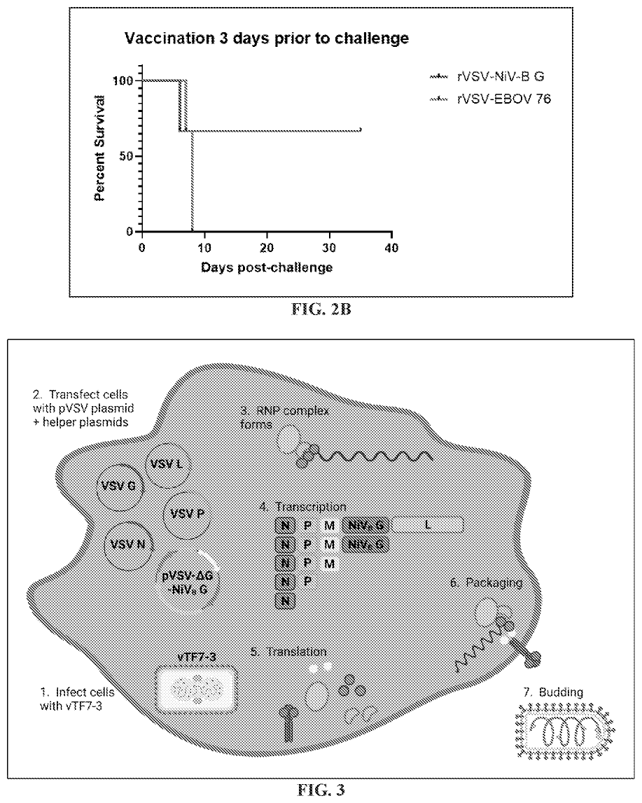

The inventors' previous version of the VSV-vectored NiV_{B} G vaccine encoded GFP downstream of the NiV_{B} G protein (FIG. 2A). This vaccine protected 100% of ferrets and AGMs from NiV_{M} and NiV_{B} when given 28 days prior to challenge^{10,11}, but the presence of a fluorescent marker would have been an obstacle to clinical trials and applications for licensure in the future. Therefore, the inventors created a version of the construct lacking GFP (FIG. 2B). A full-length antigenomic plasmid encoding the new vaccine construct was cloned as described above. Live virus was recovered, purified, and amplified to create a large stock of vaccine as discussed above and shown in FIG. 3. The viral RNA genomes of the previous rVSV-ΔG-NiV_{B} G-GFP and new rVSV-ΔG-NiV_{B} G constructs are shown in FIGS. 1A and 1B. Quantification of the virus by standard plaque assay on G* BHK cells gave a calculated titer of 4.25×10⁸ PFU/mL, confirming that the vaccine grows to similarly high titers in cell culture as other VSV constructs. The vaccine was tested and found to be negative for Mycoplasma and endotoxin contamination, and cloning borders and the NiV_{B} G gene were sequenced using Sanger sequencing. The complete sequences of viral RNA and full-length plasmid were obtained through next-generation sequencing using the NextSeq 550 system and were found to match the expected sequences.

To confirm that the vaccine construct was expressing NiV_{B} G protein in cells, an IFA as performed in Vero 76 and BHK-21 clone WI-2 cells as outlined above. As shown in FIGS. 4A to 4I, NiV_{B} G protein is strongly expressed in cells infected with G*-rVSV-ΔG-NiV_{B} G. Mock-infected wells, as well as infected wells treated with secondary antibody only as controls, are included as confirmation that the fluorescence observed is not due to autofluorescence. The high concentration of m102.4 primary antibody used contributed to some background fluorescence, as seen in the mock-infected wells in FIGS. 4C, 4F, and 4I. However, this background fluorescence disappears in the secondary-only controls (FIGS. 4A, 4D, 4G, and 4J), which are infected with G*-rVSV-ΔG-NiV_{B} G. The much brighter fluorescent signal in the G*-rVSV-ΔG-NiV_{B} G-infected wells that were incubated with both primary and secondary antibodies (FIGS. 4B, 4E, 4H, and 4K) confirms that the construct is inducing the production of NiV_{B} G protein. The results are similar between BHK-21 clone WI-2 and Vero 76 cells, although the cell number of the Vero 76 cells was lower overall. While the subcellular localization of the NiV_{B} G protein cannot be confirmed using these images, the results are similar for permeabilized and non-permeabilized cells, suggesting that the NiV_{B} G protein traffics to the cell surface for budding of rVSV-ΔG-NiV_{B} G virions.

FIGS. 1A and 1B show, FIG. 1A rVSV-ΔG-NiV_{B} G-GFP and FIG. 1B rVSV-ΔG-NiV_{B} G genomes. (FIG. 1A) Schematic of the rVSV-ΔG-NiV_{B} G-GFP RNA genome with the name of each gene indicated (VSV N, VSV P, VSV M, NiV_{B} G, GFP, and VSV L). (FIG. 1B) Schematic of the rVSV-ΔG-NiV_{B} G RNA genome with the name of each gene indicated (VSV N, VSV P, VSV, M, NiV_{B} G, and VSV L). For both panels, the genes are shown as boxes, while the intergenic regions and 3′ and 5′ untranslated regions are shown as black lines. VSV genes are shown in blue, the NiV_{B} G gene is shown in pink, and the GFP gene is shown in green. The 3′ and 5′ ends of the negative-sense viruses are indicated. GFP, green fluorescent protein; NiV_{B}, Nipah virus Bangladesh strain; RNA, ribonucleic acid; rVSV, recombinant vesicular stomatitis virus; VSV, vesicular stomatitis virus.

The rVSV-ΔG-NiV_{B} G Vaccine Protects AGMs from Lethal NiV Disease when Given Shortly Prior to Challenge with NiV_{B}

Having recovered and characterized the rVSV-ΔG-NiV_{B} G vaccine lacking GFP, the next step was to test its ability to protect AGMs from lethal challenge with NiV. The inventors have shown that NiV_{B} is uniformly lethal in the AGM model, causes more severe lung and spleen histopathology than NiV_{M}, and has a shorter therapeutic window for treatment with m102.4¹⁸. Therefore, the inventors sought to develop a novel vaccine to protect from NiV_{B}, which is also the strain currently causing outbreaks in India and Bangladesh. A prior version of the vaccine protected AGMs from NiV_{B} with one dose given 28 days prior to challenge¹¹, however, the inventor developed a vaccine that is effective in a shorter period of time between vaccination and challenge were sufficient to maintain protection in the AGM model.

The study designs for two studies with nine AGMs each are shown in FIG. 3. Animals in Study 1 were given 1×10⁷ PFU of rVSV-ΔG-NiV_{B} G (six AGMs) or 1×10⁷ PFU of rVSV-ΔG-EBOV 76 (three AGMs) seven days prior to challenge with 5×10³ PFU of NiV_{B} given i.n./i.t. Animals in Study 2 were given the same doses of vaccine three days prior to challenge with the same dose of NiV_{B}. The animals receiving the non-specific EBOV vaccine served as controls and succumbed to NiV disease by day 9 post-challenge. Survival curves are shown in FIGS. 2A and 2B. When given rVSV-ΔG-NiV_{B} G seven days prior to NiV_{B} challenge, 100% of the animals survived to the study endpoint. Encouragingly, 67% of the animals receiving rVSV-ΔG-NiV_{B} G three days prior to NiV_{B} challenge also survived. Therefore, the inventors have shown in a robust, 100% lethal animal model that faithfully recapitulates human NiV disease that the rVSV-ΔG-NiV_{B} G vaccine protects well when given shortly before exposure. These results are encouraging because a vaccine that works quickly is essential for controlling an ongoing outbreak of NiV disease.

FIGS. 4A to 4K: The rVSV-ΔG-NiV_{B} G construct expresses NiV_{B} G protein. Results of an IFA in BHK-21 clone WI-2 (panels A through E) and Vero 76 cells (panels F through K) infected with G*-rVSV-ΔG-NiV_{B} G (panels A, B, D, E, G, H, J, and K) or mock-infected with growth medium only (panels C, F, and I). Cells were permeabilized with 0.5% Triton™ X-100 (panels B and C and F through H) or not permeabilized (panels C through E and I through K) and then incubated with human m102.4 primary antibody (panels B, C, E, F, H, I, and K) followed by a secondary antibody against human IgG conjugated to a green fluorescent fluorophore (all panels). BHK, baby hamster kidney; IFA, immunofluorescence assay; IgG, immunoglobulin G; NiV_{B}, Nipah virus Bangladesh strain; rVSV, recombinant vesicular stomatitis virus.

FIGS. 5A and 5B show the study designs for experimental challenge studies in AGMs. Experimental design for (A) Study 1, in which AGMs were vaccinated seven days prior to challenge with NiV_{B}, and (B) Study 2, in which AGMs were vaccinated three days prior to challenge. The red triangle indicates vaccination with the non-specific rVSV-ΔG-EBOV 76 vector, while the blue triangle denotes vaccination with the rVSV-ΔG-NiV_{B} G vector developed for these studies. Study days relative to challenge with 5×10⁵ PFU of NiV_{B} are shown below the horizontal black line, with days of blood collection indicated with arrows. AGM, African green monkey; EBOV, Zaire ebolavirus; NiV_{B}, Nipah virus Bangladesh strain; PFU, plaque-forming units; rVSV, recombinant vesicular stomatitis virus.

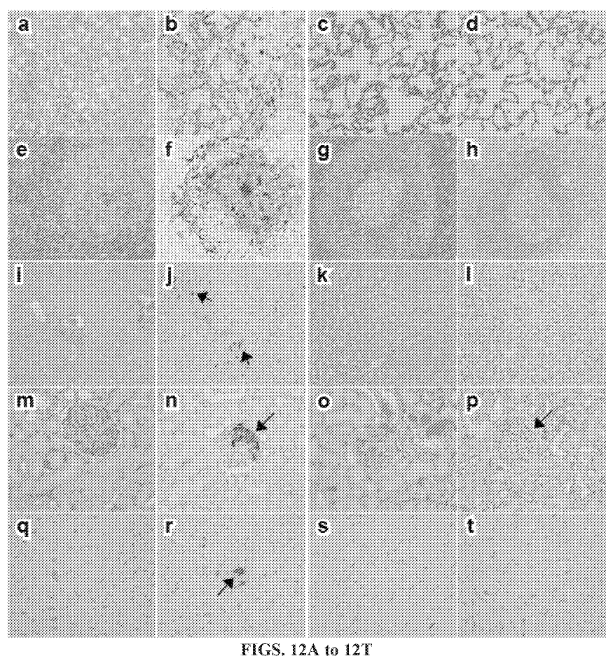

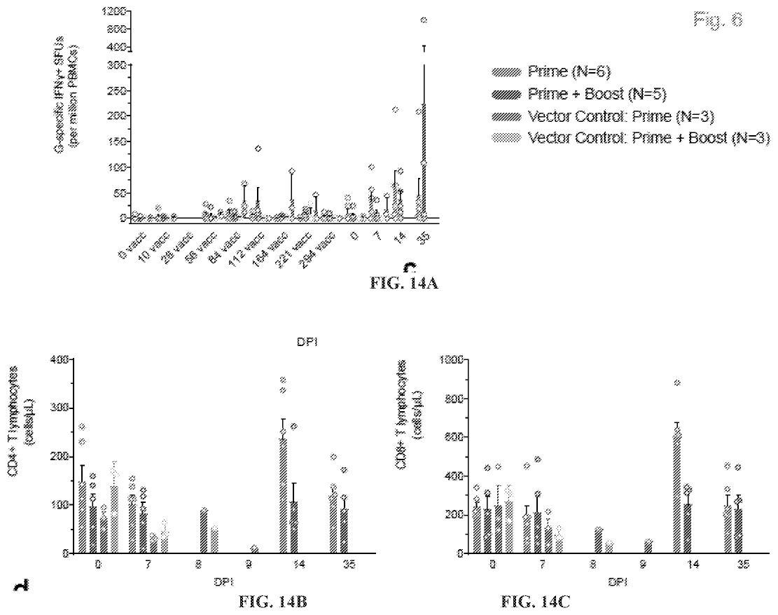

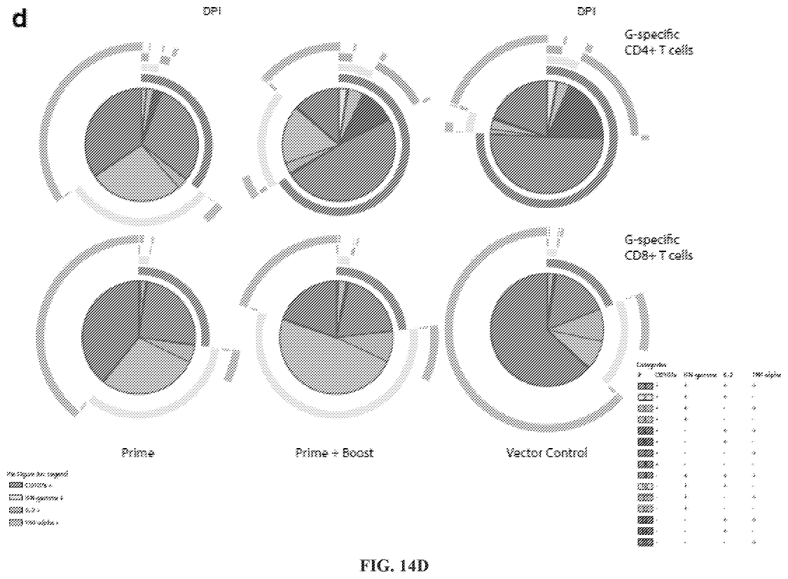

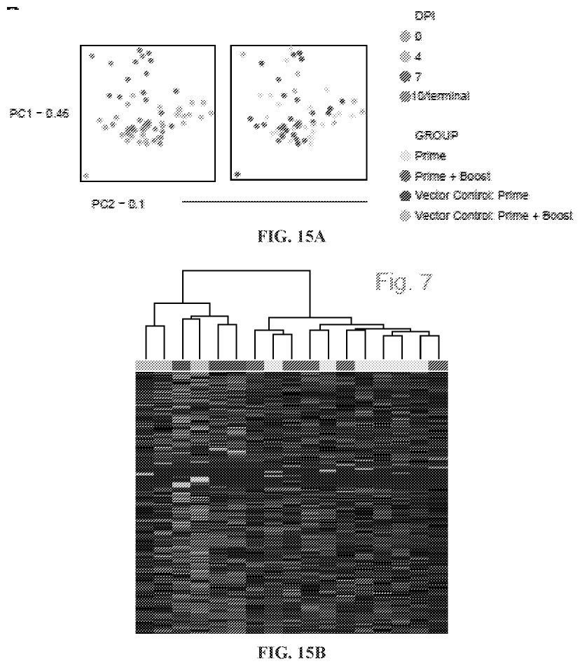

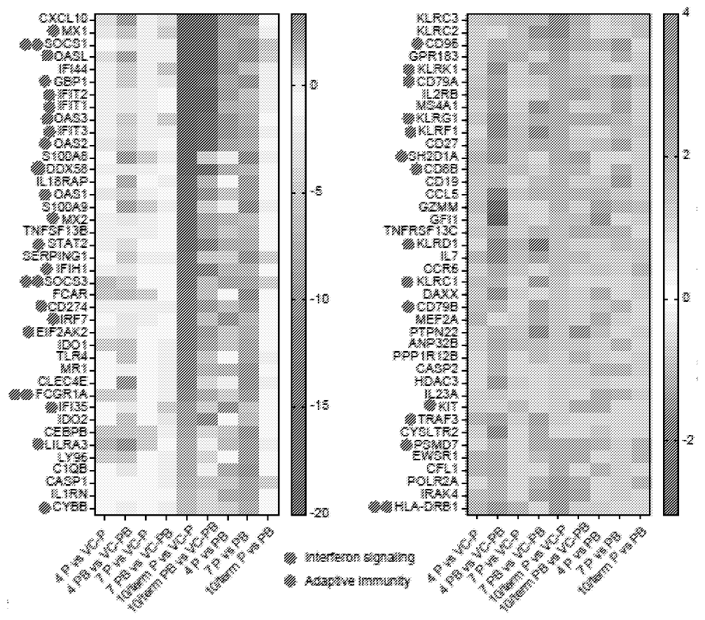

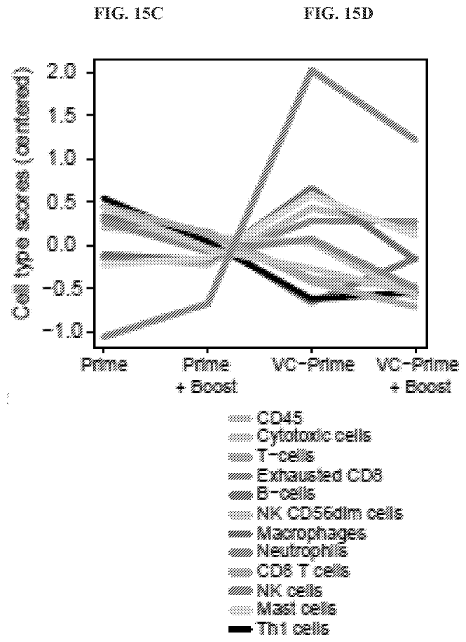

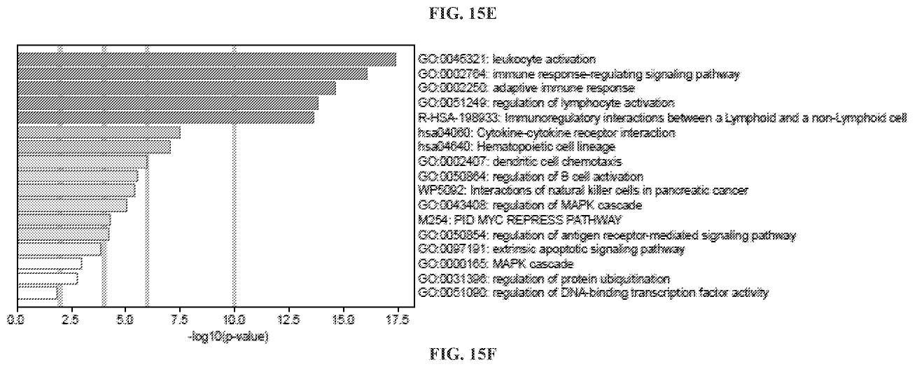

Clinical signs and observations for Study 1 are shown in Table 1. Importantly, animals that received the rVSV-ΔG-NiV_{B} G vaccine seven days prior to challenge exhibited no clinical signs other than decreased appetite throughout the study. Clinical pathology was minimal, although some subjects had transient increases in CRP, indicating inflammation, and some changes to blood counts outside of their baseline values (Table 1). Conversely, animals that received the rVSV-ΔG-EBOV 76 vaccine seven days prior to challenge developed dyspnea and depression prior to reaching humane euthanasia criteria between seven and nine days post-infection (dpi). Their respiratory rates increased sharply just prior to euthanasia, and significantly increased respiratory rates were a reliable marker of rapid clinical decline in infected animals (FIGS. 12A-12T, 14A-14C). These animals had decreased levels of white blood cells, lymphocytes, and thrombocytes, indicating immune suppression associated with hemorrhagic disease. They also had increased levels of neutrophils and CRP, which are hallmarks of inflammation. In short, animals in the rVSV-ΔG-EBOV 76 control group were unable to control their NiV infection and succumbed to NiV disease with its trademark clinical markers, while subjects in the rVSV-ΔG-NiV_{B} G group effectively controlled the NiV infection and survived to the study endpoint without noticeable clinical signs.

Clinical signs and observations for Study 2 are shown in Table 2. As in Study 1, animals receiving the rVSV-ΔG-EBOV 76 vaccine developed respiratory and systemic signs consistent with NiV disease, such as tachypnea, dyspnea, and depression, and reached euthanasia criteria at seven or eight dpi. However, two of the animals vaccinated with rVSV-ΔG-NiV_{B} G three days prior to challenge also reached euthanasia criteria at six dpi. The four remaining animals receiving the NiV_{B}-specific vaccine in Study 2 developed respiratory signs and transient elevated CRP but recovered and survived to the study endpoint at 35 dpi. These subjects also had temporary increases in inflammatory immune cells, such as neutrophils, and temporary decreases in thrombocytes. These clinical findings were similar to those observed in the animals that succumbed to NiV_{B} infection, although they were less severe, and these animals recovered (Table 2). Unlike Study 1, in which animals vaccinated with rVSV-ΔG-NiV_{B} G did not develop clinical signs of NiV disease, all AGMs in Study 2 developed clinical signs consistent with NiV disease. However, the severity was lower in four out of the six animals in the NiV_{B}-vaccinated group, and these animals recovered, with their clinical pathology values returning to normal by around 10 dpi (Table 2).

Table 1, next page. Clinical disease and findings in AGMs vaccinated seven days prior to experimental infection with NiV_{B}

TABLE 1

| NHP | Sex | Vaccine | Clinical signs/outcome | Hematology/clinical pathology findings |
|---|---|---|---|---|
| C7-1 | Female | rVSV-ΔG- | Tachypnea (d 7); dyspnea (d 7); anorexia | Leukopenia (d 0); lymphopenia (d 0, 7); monocytopenia |
| | | EBOV 76 | (d 7); depression (d 7); hypothermia (d 7). | (d 0, 4, 7); eosinopenia (d 0, 4); basopenia (d 0, 4, 7); |
| | | | Subject euthanized (d 7). | thrombocytopenia (d 7); neutrophilia (d 7); ↑ CRP (d 7). |
| C7-2 | Female | rVSV-ΔG- | Dyspnea (d 7); shallow breathing (d 8); | Leukopenia (d 8); lymphopenia (d 0 ,7, 8); eosinopenia |
| | | EBOV 76 | severe depression (d 8); fever (d 7); | (d 0, 4, 7, 8); basopenia (d 0, 4, 7, 8); thrombocytopenia |
| | | | hypothermia (d 8). Subject euthanized | (d 7, 8); hyperglycemia (d 8); ↑ BUN (d 8); ↓ CRE (d 0); ↑ |
| | | | (d 8). | ALT (d 7, 8); ↑ AST (d 8); ↑ CRP (d 7); ↑↑ CRP (d 8). |
| C7-3 | Male | rVSV-ΔG- | Tachypnea (d 9); shallow breathing (d 9); | Lymphopenia (d 9); eosinopenia (d 0, 4, 7); basopenia |
| | | EBOV 76 | dyspnea (d 9); decreased appetite (d 8, 9); | (d 0, 4, 7, 9); thrombocytopenia (d 9); monocytosis (d 9); |
| | | | depression (d 9). Subject euthanized (d 9). | neutrophilia (d 0, 9); ↓ CRE (d 4); ↑ CRP (d 9). |
| V7-1 | Female | rVSV-ΔG- | Decreased appetite (d 1, 5-11, 20, 22); | Leukopenia (d 35); lymphopenia (d 35); monocytopenia |
| | | NiV_{B} G | hypothermia (d 35). Subject survived to | (d 35); neutropenia (d 4); eosinopenia (d 0, 7); basopenia |
| | | | study endpoint (d 35). | (d 0, 7); thrombocytopenia (d 4); monocytosis (d 4); |
| | | | | eosinophilia (d 4); basophilia (d 4, 15); ↑ CRE (d 7); ↓ CRE |
| | | | | (d 15, 35). |
| V7-2 | Female | rVSV-ΔG- | Decreased appetite (d 16); fever (d 4); | Leukopenia (d 28, 35); monocytopenia (d 0, 4, 7, 10, 15, |
| | | NiV_{B} G | hypothermia (d 35). Subject survived to | 21, 28, 35); neutropenia (d 28, 35); eosinopenia (d 0, 4, 7, |
| | | | study endpoint (d 35). | 10, 15, 21, 28, 35); basopenia (d 28, 35); neutrophilia |
| | | | | (d 4); thrombocytosis (d 10); ↓ CRE (d 35); ↑ CRP (d 4). |
| V7-3 | Female | rVSV-ΔG- | Decreased appetite (d 1, 5, 8, 11, 16, 18-24, | Leukocytosis (d 7, 10); monocytosis (d 4, 7, 10, 15, 21, |
| | | NiV_{B} G | 27-32, 34, 35); hypothermia (d 35). | 35); neutrophilia (d 4, 7, 10); eosinophilia (d 4, 7, 10, 15); |
| | | | Subject survived to study endpoint (d 35). | basophilia (d 4, 7, 10, 15); ↓ CRE (d 35). |
| V7-4 | Male | rVSV-ΔG- | None. Subject survived to study endpoint | Leukocytosis (d 4, 7, 10, 15); lymphocytosis (d 0, 7, 10, |
| | | NiV_{B} G | (d 35). | 15); neutrophilia (d 0, 4, 7, 10, 15, 21, 28, 35); basophilia |
| | | | | (d 15); monocytopenia (d 0, 4, 7, 10, 15, 21, 28, 35); |
| | | | | eosinopenia (d 28); basopenia (d 21, 28). |
| V7-5 | Male | rVSV-ΔG- | Hypothermia (d 35). Subject survived to | Monocytopenia (d 0, 4, 7, 10, 15, 28, 35); neutrophilia |
| | | NiV_{B} G | study endpoint (d 35). | (d 0, 4, 7, 10, 15, 21, 28, 35); ↑ CRP (d 4). |
| V7-6 | Male | rVSV-ΔG- | None. Subject survived to study endpoint | Monocytopenia (d 0, 4, 7, 10, 15, 21, 28, 35); eosinopenia |
| | | NiV_{B} G | (d 35). | (d 7); neutrophilia (d 0, 4, 7, 10, 15, 21, 28, 35); |
| | | | | basophilia (d 0, 21, 35); ↓ CRE (d 0, 10, 15, 21); ↑ CRP |
| | | | | (d 4). |

Days after NiV_{B} challenge are in parentheses. All reported findings are in comparison to baseline (d-7) values. Decreased appetite is defined as some food but not all food consumed from the previous day. Anorexia is defined as no food consumed from the previous day. Fever is defined as a temperature more than 2.5° F. over baseline, or at least 1.5° F. over baseline and ≥103.5° F. Hypothermia is defined as a temperature ≤3.5° F. below baseline. Leukopenia, lymphopenia, monocytopenia, neutropenia, eosinopenia, basopenia, and thrombocytopenia are defined by a ≥35% drop in numbers of white blood cells, lymphocytes, monocytes, neutrophils, eosinophils, basophils, or platelets, respectively. Leukocytosis, lymphocytosis, monocytosis, neutrophilia, eosinophilia, basophilia, and thrombocytosis are defined by a 100% or greater increase in numbers of white blood cells, lymphocytes, monocytes, neutrophils, eosinophils, basophils, or thrombocytes, respectively. Hyperglycemia is defined as a 100% or greater increase in levels of glucose. Increases and decreases in BUN, CRE, ALT, AST, and CRP were graded on the following scale: ↑ = 1- to 5-fold increase, ↑↑ = >5- to 10-fold increase, ↑↑↑ = >10- to 20-fold increase, ↑↑↑↑ = >20-fold increase, ↓ = ≥50% decrease. ALT, alanine aminotransferase; AST, aspartate aminotransferase; BUN, blood urea nitrogen; CRE, creatinine; CRP, C-reactive protein.

TABLE 2
Clinical disease and findings in AGMs vaccinated three days prior to experimental infection with NiV_{B}

| NHP | Sex | Vaccine | Clinical signs/outcome | Hematology/clinical pathology findings |
|---|---|---|---|---|
| C3-1 | Female | rVSV-ΔG- | Dyspnea (d 7, 8); anorexia (d 8); | Leukocytosis (d 8); monocytosis (d 0, 4, 8); neutrophilia |
| | | EBOV 76 | depression (d 8); fever (d 7); hypothermia | (d 7, 8); lymphocytopenia (d 7, 8); neutropenia (d 4); |
| | | | (d 8). Subject euthanized (d 8). | eosinopenia (d 0, 4, 7); basopenia (d 4, 7); |
| | | | | thrombocytopenia (d 8); ↑ CRE (d 8); ↑ ALT (d 8); ↑ CRP |
| | | | | (d 7); ↑↑ CRP (d 8). |
| C3-2 | Male | rVSV-ΔG- | Tachypnea (d 8); dyspnea (d 8); decreased | Lymphopenia (d 8); monocytopenia (d 7); eosinopenia |
| | | EBOV 76 | appetite (d 5-7); anorexia (d 8); depression | (d 0, 4, 7, 8); basopenia (d 0, 8); neutrophilia (d 8); |
| | | | (d 8). Subject euthanized (d 8). | hypoalbuminemia (d 8); ↑↑ALT (d 0, 4, 7, 8); ↑AST (d 8); |
| | | | | hypoamylasemia (d 8); ↑ CRP (d 7); ↑↑ CRP (d 8). |
| C3-3 | Male | rVSV-ΔG- | Taychypnea (d 7); dyspnea (d 7); | Lymphopenia (d 7); eosinopenia (d 0, 4, 7); basopenia |
| | | EBOV 76 | decreased appetite (d 5-7); depression | (d 0, 4, 7); thrombocytopenia (d 7); monocytosis (d 7); ↑ |
| | | | (d 7). Subject euthanized (d 7). | CRP (d 7). |
| V3-1 | Female | rVSV-ΔG- | Tachypnea (d 5, 6); dyspnea (d 6); anorexia | Leukocytosis (d 6); monocytosis (d 0, 4, 6); neutrophilia |
| | | NiV_{B} G | (d 5, 6); depression (d 6); fever (d 4); | (d 6); eosinophilia (d 6); lymphocytopenia (d 4); |
| | | | hypothermia (d 6). Subject euthanized | eosinopenia (d 4); basopenia (d 4); thrombocytopenia |
| | | | (d 6). | (d 4, 6); hyperglycemia (d 6); ↑ CRE (d 6); |
| | | | | hypoalbuminemia (d 6); ↑↑ ALT (d 6); ↑↑ AST (d 6); |
| | | | | ↑↑↑ CRP (d 6). |
| V3-2 | Female | rVSV-ΔG- | Tachypnea (d 6); dyspnea (d 6); decreased | Leukocytosis (d 6); monocytosis (d 0, 6); neutrophilia |
| | | NiV_{B} G | appetite (d 5, 6); depression (d 6); fever | (d 6); eosinophilia (d 6); lymphocytopenia (d 4); |
| | | | (d 4); hypothermia (d 6); seizures (d 6); | basopenia (d 4, 6); thrombocytopenia (d 6); |
| | | | epistaxis (d 6). Subject euthanized (d 6). | hyperglycemia (d 6); ↓ CRE (d 4); ↑ CRE (d 6); |
| | | | | hypoalbuminemia (d 6); hypoproteinemia (d 6); ↑ CRP |
| | | | | (d 4, 6). |
| V3-3 | Female | rVSV-ΔG- | Tachypnea (d 5, 6, 7); decreased appetite | Leukopenia (d 0); lymphopenia (d 4, 35); monocytopenia |
| | | NiV_{B} G | (d 5-9, 11, 12). Subject survived to study | (d 0, 21, 35); neutropenia (d 0, 21); eosinopenia (d 0, 7, |
| | | | endpoint (d 35). | 14, 21, 28); basopenia (d 7, 28); monocytosis (d 14); |
| | | | | neutrophilia (d 4); eosinophilia (d 10); basophilia (d 10); |
| | | | | hypoglycemia (d 35); hypoalbuminemia (d 7); ↑ ALT |
| | | | | (d 35); ↑ AST (d 7); hypoamylasemia (d 4); ↑ CRP (d 4, 7). |
| V3-4 | Male | rVSV-ΔG- | Tachypnea (d 8); dyspnea (d 7); decreased | Leukocytosis (d 10, 14); lymphocytosis (d 14); |
| | | NiV_{B} G | appetite (d 5-11); fever (d 7). Subject | neutrophilia (d 4, 7, 10, 14, 21); basophilia (d 14); |
| | | | survived to study endpoint (d 35). | eosinopenia (d 0, 4, 10, 35); basopenia (d 4, 7, 10); |
| | | | | thrombocytopenia (d 7); ↑ AST (d 10); hypoamylasemia |
| | | | | (d 4, 7, 14, 21, 28, 35); ↑↑↑ CRP (d 7). |
| V3-5 | Male | rVSV-ΔG- | Decreased appetite (d 5-8; 11, 15, 22, 29, | Monocytosis (d 7, 10, 14); neutrophilia (d 10); |
| | | NiV_{B} G | 30, 33, 35); fever (d 4, 7). Subject survived | eosinophilia (d 35); eosinopenia (d 0, 4, 10, 21); |
| | | | to study endpoint (d 35). | basopenia (d 0, 4, 7, 10, 21); thrombocytopenia (d 7, 35); |
| | | | | ↑ ALT (d 0, 35); ↑↑ AST (d 35); hypoamylasemia (d 4); ↑ |
| | | | | CRP (d 7). |
| V3-6 | Male | rVSV-ΔG- | Tachypnea (d 6, 7); dyspnea (d 8); | Leukocytosis (d 10, 14); lymphocytosis (d 14); |
| | | NiV_{B} G | decreased appetite (d 5-7, 9-11, 15, 22, 24, | monocytosis (d 10); neutrophilia (d 7, 10, 14); |
| | | | 25, 29, 30, 32-34); anorexia (d 8); | lymphocytopenia (d 7); monocytopenia (d 0, 4, 35); |
| | | | hypothermia (d 10). Subject survived to | eosinopenia (d 7); basopenia (d 4, 7); thrombocytopenia |
| | | | study endpoint (d 35). | (d 4, 7); ↑ CRP (d 7). |

Days after NiV_{B} challenge are in parentheses. All reported findings are in comparison to baseline (d-3) values. Decreased appetite is defined as some food but not all food consumed from the previous day. Anorexia is defined as no food consumed from the previous day. Fever is defined as a temperature more than 2.5° F. over baseline, or at least 1.5° F. over baseline and ≥103.5° F. Hypothermia is defined as a temperature ≤3.5° F. below baseline. Leukopenia, lymphopenia, monocytopenia, neutropenia, eosinopenia, basopenia, and thrombocytopenia are defined by a ≥35% drop in numbers of white blood cells, lymphocytes, monocytes, neutrophils, eosinophils, basophils, or platelets, respectively. Leukocytosis, lymphocytosis, monocytosis, neutrophilia, eosinophilia, and basophilia are defined by a 100% or greater increase in numbers of white blood cells, lymphocytes, monocytes, neutrophils, eosinophils, or basophils, respectively. Hyperglycemia is defined as a 100% or greater increase in levels of serum glucose. Hypoglycemia is defined by a a ≥25% decrease in levels of serum glucose. Hypoalbuminemia is defined by a ≥25% decrease in levels of albumin. Hypoproteinemia is defined by a ≥25% decrease in levels of total protein. Hypoamylasemia is defined by a ≥25% decrease in levels of serum amylase. Increases and decreases in CRE, ALT, AST, and CRP were graded on the following scale: ↑ = 1- to 5-fold increase, ↑↑ = >5- to 10-fold increase, ↑↑↑ = >10- to 20-fold increase, ↑↑↑↑ = >20-fold increase, ↓ = ≥50% decrease. ALT, alanine aminotransferase; AST, aspartate aminotransferase; CRE, creatinine; CRP, C-reactive protein.

FIGS. 2A and 2B shows the survival curves following challenge of vaccinated AGMs with NiV_{B}. Kaplan-Meier survival curves for AGMs vaccinated with rVSV-dG-NiV_{B} G (blue line) or rVSV-dG-EBOV 76 (red line)(A) seven days or (B) three days prior to challenge with NiV_{B}. AGM, African green monkey; EBOV, Zaire ebolavirus; NiV_{B}, Nipah virus Bangladesh strain; rVSV, recombinant vesicular stomatitis virus.

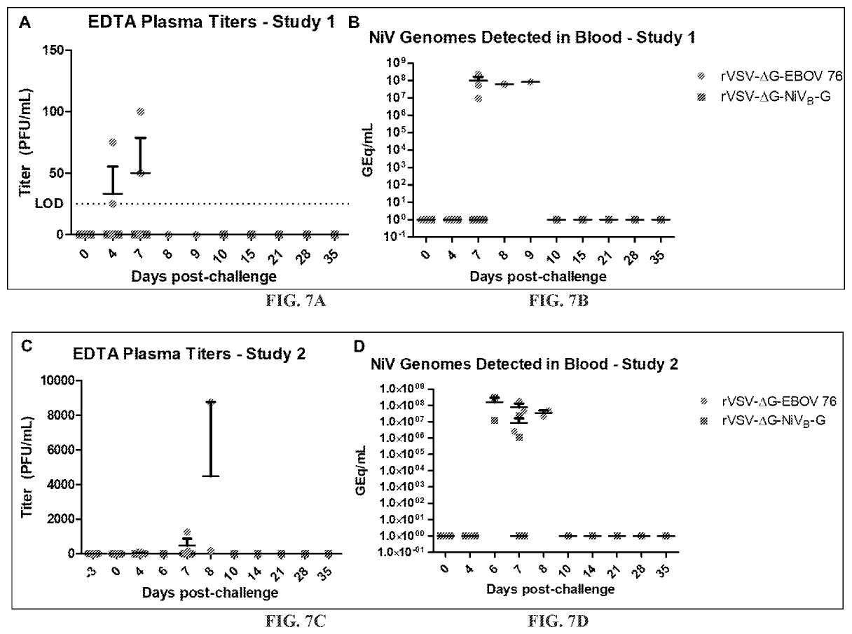

Viremia, as measured in live, recoverable virus by plaque assay and as GEq by qRT-PCR, is shown in FIGS. 7A to 7D. During Study 1, no animals vaccinated with rVSV-ΔG-NiV_{B} G developed viremia detectable by either method. Conversely, control animals vaccinated with rVSV-ΔG-EBOV 76 developed detectable viremia (two animals by plaque assay and all three animals by qRT-PCR) shortly prior to terminal endpoint (FIGS. 7A and 7B). Results were similar in Study 2. All animals that succumbed to NiV disease in either vaccination group developed detectable viremia by plaque assay and qRT-PCR shortly prior to euthanasia (FIGS. 7C and 7D). Additionally, two of the four survivors vaccinated with rVSV-ΔG-NiV_{B} G developed viremia detectable by qRT-PCR, although it resolved by 10 dpi (FIG. 4-7D). No virus was detected by either method prior to day six or after day eight post-challenge.

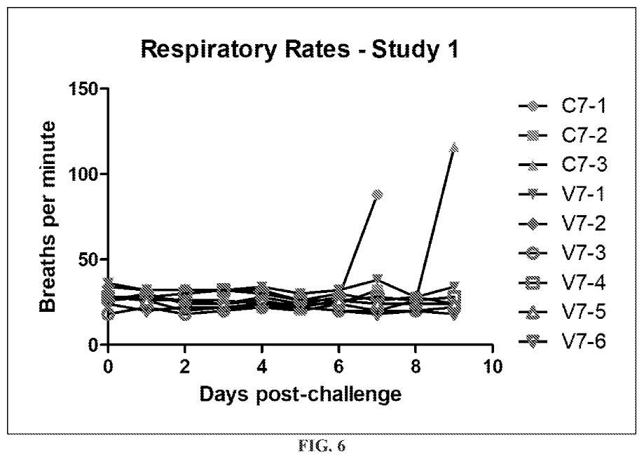

FIG. 6: Respiratory rates following challenge of AGMs with NiV_{B}. Line graph of respiratory rates in breaths per minute tracked daily throughout Study 1 for animals vaccinated with rVSV-ΔG-NiV_{B} G (blue symbols) or rVSV-ΔG-EBOV 76 (red symbols) seven days prior to challenge with NiV_{B}. Animal IDs are shown in the legend to the right of the graph. AGM, African green monkey; EBOV, Zaire ebolavirus; NiV_{B}, Nipah virus Bangladesh strain; rVSV, recombinant vesicular stomatitis virus.

FIGS. 7A to 7D show the viremia in vaccinated AGMs after challenge with NiV_{B}. Live virus isolated from EDTA plasma by plaque assay (FIGS. FIGS. 7A and 7C) or viral RNA detected in whole blood by qRT-PCR specific for NiV_{B} (panels 7B and 7D) for AGMs challenged with NiV_{B} seven days after vaccination (FIGS. 7A and 7B) or three days after vaccination (FIGS. 7C and 7D). Groups vaccinated with rVSV-ΔG-EBOV 76 are represented by red circles, and groups vaccinated with rVSV-ΔG-NiV_{B} G are represented by blue circles. Horizontal lines represent the mean of the values for all members of the group at each timepoint, and error bars represent the SEM. The LOD for the plaque assays is 25 PFU; qRT-PCR values are reported as 1 GEq/mL if they were below the LOD. AGM, African green monkey; EBOV, Zaire ebolavirus; EDTA, ethylenediaminetetraacetic acid; GEq, genome equivalent; LOD, limit of detection; mL, milliliter; NiV_{B}, Nipah virus Bangladesh strain; PFU, plaque-forming units; qRT-PCR, quantitative reverse transcriptase-polymerase chain reaction; SEM, standard error of the mean; rVSV, recombinant vesicular stomatitis virus.

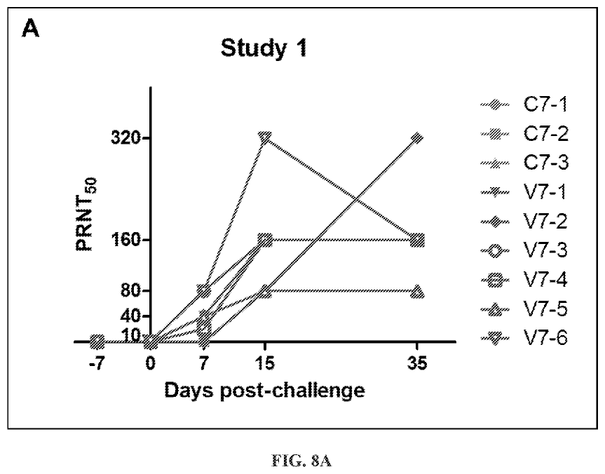

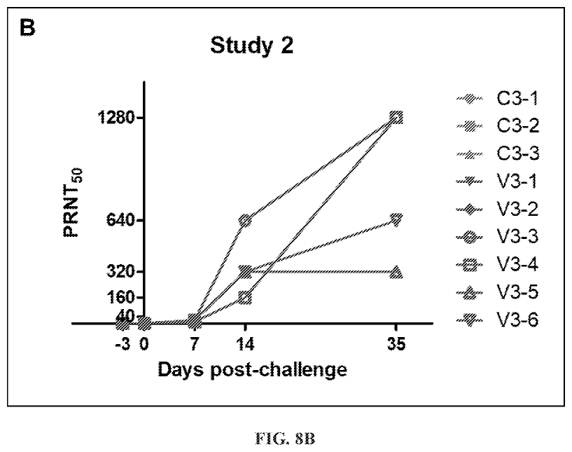

Since the rVSV-ΔG-NiV_{B} G vaccine protected AGMs when given shortly prior to challenge with NiV_{B}, the inventors wanted to investigate the immunological phenotype of the survivors. FIGS. 8A and 8B show the results of PRNT assays to quantify neutralizing antibody to NiV_{B} in the EDTA plasma of all subjects. All survivors in both Study 1 (animals V7-1 through V7-6) and Study 2 (animals V3-3 through V3-6) developed neutralizing antibodies that were detectable beginning at day seven post-challenge. Interestingly, animals in Study 2 developed higher neutralizing antibody titers than subjects in Study 1. The inventors hypothesized that this difference may be due to replication of the NiV_{B} inoculum in these animals due to incomplete control of the viral infection because these subjects were vaccinated only three days prior to challenge instead of a full week (as in Study 1), so NiV-specific immunological responses may have taken a few days after challenge to develop. However, early replication in tissues could not be confirmed in these studies because animals were not sacrificed prior to humane endpoint or the end of the study. None of the animals that succumbed to NiV disease developed detectable neutralizing antibodies, regardless of vaccine group.

FIGS. 8A and 8B show PRNT₅₀ results for AGMs vaccinated prior to challenge with NiV_{B}. PRNT₅₀ values from EDTA plasma, reported as reciprocal dilutions at which plaque counts were reduced by 50% compared to control wells, for animals vaccinated with rVSV-ΔG-NiV_{B} G (blue symbols) or rVSV-ΔG-EBOV 76 (red symbols) (A) seven days or (B) three days prior to challenge with NiV_{B}. Animal IDs are shown in the legend to the right of the graph. AGM, African green monkey; EBOV, Zaire ebolavirus; EDTA, ethylenediaminetetraacetic acid; NiV_{B}, Nipah virus Bangladesh strain; PRNT, plaque reduction neutralization test; rVSV, recombinant vesicular stomatitis virus.

It is shown herein that the rVSV-ΔG-NiV_{B} G vaccine is highly effective at protecting AGMs, an excellent model of NiV disease in humans, from a dose of 1×10⁷ PFU of NiV_{B} given by a biologically plausible route either seven days or three days after vaccination. Clinical signs of NiV disease, inflammatory and hematological markers, and viremia were transient in survivors, and no lingering nor late-onset neurological signs were observed. All surviving animals developed neutralizing antibodies to NiV_{B}, although these antibodies were not detectable until the window during which non-survivors were beginning to succumb to NiV disease.

The rVSV-ΔG-NiV_{B} G vaccine has an ideal profile for licensure and deployment during an outbreak of NiV disease. Firstly, it is expected to be safe for use in humans because VSV-based vaccines have been shown to be safe in the past, as evidenced by the licensure of Ervebo, Merck's VSV-vectored Ebola vaccine. VSV has mild pathogenicity in humans, this construct lacks VSV G, which is the main driver of virulence. Because the rVSV-ΔG-NiV_{B} G construct encodes only NiV_{B} G and is not an attenuated NiV, it cannot revert to virulent NiV_{B}. As a negative-sense, single-stranded RNA virus, it cannot reassort and cannot integrate into the host genome. Furthermore, the fact that rVSV-ΔG-NiV_{B} G is a single-cycle vaccine which cannot replicate without complementation and yet is still immunogenic, as evidenced by the development of neutralizing antibodies to NiV_{B} in surviving AGMs here and by previous studies with replication-incompetent rVSV constructs¹⁶, enhances its safety profile.

While rVSV-ΔG-NiV_{B} G requires storage at −80° C. and maintenance of cold chain to ensure efficacy, Ervebo has the same storage and transportation constraints and has been used effectively in recent outbreaks of Ebola in the Democratic Republic of the Congo. Multiple technologies have been developed to transport the vaccine to remote, resource-limited areas while maintaining cold chain, and stability studies could be undertaken to determine how long the vaccine could be stored at, for example, 4° C. while retaining its potency. Studies are underway to determine the dose of rVSV-ΔG-NiV_{B} G vaccine needed to protect AGMs from lethal NiV_{B} challenge, but the construct easily grows to titers exceeding 1×10⁸ PFU per mL in just 24 hours, so manufacturing of many doses in a short timeframe should be very achievable.

The control vaccine used in these studies was also a VSV-vectored vaccine, although it is fully replicative because the glycoprotein is the only protein required for entry of EBOV into cells. A non-replicating, non-specific rVSV vector could have been used as a more direct control instead, or the control group could have been administered PBS instead of a vaccine. However, the replicating VSV-vectored vaccine was the most robust control group these studies could have had. A previous study showed that 100% of hamsters vaccinated one day prior to challenge with a replicating rVSV-vectored NiV vaccine survived challenge with NiV_{M}; partial survival was observed in additional groups that were vaccinated on the day of challenge (four out of six animals) or one day after challenge (one out of six animals)¹⁴. However, the authors attributed this protection to innate responses or responses to the rVSV vector itself rather than specific responses to the NiV component of the vaccines, as animals vaccinated with a non-specific rVSV-EBOV-GP control vector survived challenge with NiV_{M} when vaccinated one day prior to challenge (three out of six animals) or day of challenge (two out of six animals)¹⁴. In this example, using a very similar non-specific control vector but with a more relevant model of NiV disease in humans (AGMs) and more virulent strain of NiV (NiV_{B}), 100% of the animals vaccinated with the rVSV-ΔG-EBOV 76 control vector succumbed to NiV disease. Therefore, it is clear from this study that survival was based on NiV-specific responses and not solely on innate nor VSV-specific immunity because the replicating vaccine used to vaccinate controls should have elicited equal or greater innate and VSV-specific responses as rVSV-ΔG-NiV_{B} G¹⁶. All animals vaccinated with rVSV-ΔG-EBOV 76 succumbed to NiV disease during the normal window for this model and dose. Therefore, innate and VSV-specific immunity did not contribute to survival in these studies.

Late-onset encephalitis and other neurological problems are a concern with NiV, and neurovirulence is a possibility with wild-type VSV and has been investigated for other VSV-vectored constructs. Ensuring that these concerns are not likely to be exacerbated by the rVSV-dG-NiV_{B} G construct will be of the utmost importance for continuing studies with the construct. None of the animals in either of the studies presented in this chapter developed late-onset neurological signs, which have occurred between 28 and 35 dpi in previous NiV studies performed by the present inventors. While these were short-term studies, lasting only five weeks, this shows that the vaccine is not just suppressing viral replication to a low level and then allowing it to begin replicating later. The rVSV-ΔG constructs lack the protein that allows VSV neurovirulence (VSV G), and the safety of the rVSV-ΔG-NiV_{B} G vector is enhanced by the fact that it encodes just one of the two proteins necessary for NiV viral entry and therefore neurovirulence. Therefore, the rVSV-ΔG-NiV_{B} G vaccine is safe and free of neurovirulence concerns.

In conclusion, rVSV-ΔG-NiV_{B} G is a safe, immunogenic, and effective vaccine which protected AGMs from a high dose of NiV_{B} given shortly after vaccination. These studies are an encouraging first step in showing the safety and potential efficacy of the vaccine in an outbreak scenario. Studies are ongoing to evaluate protective immune responses to the vaccine, minimum dose needed for efficacy, and durability of vaccine-induced immune responses. Future studies will focus on standardizing manufacturing with an eye towards licensure. A fast-acting and effective vaccine is urgently needed for NiV, which still causes outbreaks in India and Bangladesh nearly every year with high CFRs, thus VSV-ΔG-NiV_{B} G will be an invaluable tool in the control of this deadly pathogen.

pVSV-ΔG-NiV_{B} G
SEQ ID NO: 1
ACGAAGACAAACAAACCATTATTATCATTAAAAGGCTCAGGAGAAACTTTAACAGTA
ATCAAAATGTCTGTTACAGTCAAGAGAATCATTGACAACACAGTCGTAGTTCCAAAAC
TTCCTGCAAATGAGGATCCAGTGGAATACCCGGCAGATTACTTCAGAAAATCAAAGG
AGATTCCTCTTTACATCAATACTACAAAAAGTTTGTCAGATCTAAGAGGATATGTCTA
CCAAGGCCTCAAATCCGGAAATGTATCAATCATACATGTCAACAGCTACTTGTATGGA
GCATTAAAGGACATCCGGGGTAAGTTGGATAAAGATTGGTCAAGTTTCGGAATAAAC
ATCGGGAAAGCAGGGGATACAATCGGAATATTTGACCTTGTATCCTTGAAAGCCCTGG
ACGGCGTACTTCCAGATGGAGTATCGGATGCTTCCAGAACCAGCGCAGATGACAAAT
GGTTGCCTTTGTATCTACTTGGCTTATACAGAGTGGGCAGAACACAAATGCCTGAATA
CAGAAAAAAGCTCATGGATGGGCTGACAAATCAATGCAAAATGATCAATGAACAGTT
TGAACCTCTTGTGCCAGAAGGTCGTGACATTTTTGATGTGTGGGGAAATGACAGTAAT
TACACAAAAATTGTCGCTGCAGTGGACATGTTCTTCCACATGTTCAAAAAACATGAAT
GTGCCTCGTTCAGATACGGAACTATTGTTTCCAGATTCAAAGATTGTGCTGCATTGGC
AACATTTGGACACCTCTGCAAAATAACCGGAATGTCTACAGAAGATGTAACGACCTG
GATCTTGAACCGAGAAGTTGCAGATGAAATGGTCCAAATGATGCTTCCAGGCCAAGA
AATTGACAAGGCCGATTCATACATGCCTTATTTGATCGACTTTGGATTGTCTTCTAAGT
CTCCATATTCTTCCGTCAAAAACCCTGCCTTCCACTTCTGGGGGCAATTGACAGCTCTT
CTGCTCAGATCCACCAGAGCAAGGAATGCCCGACAGCCTGATGACATTGAGTATACA
TCTCTTACTACAGCAGGTTTGTTGTACGCTTATGCAGTAGGATCCTCTGCCGACTTGGC
ACAACAGTTTTGTGTTGGAGATAACAAATACACTCCAGATGATAGTACCGGAGGATTG
ACGACTAATGCACCGCCACAAGGCAGAGATGTGGTCGAATGGCTCGGATGGTTTGAA
GATCAAAACAGAAAACCGACTCCTGATATGATGCAGTATGCGAAAAGAGCAGTCATG
TCACTGCAAGGCCTAAGAGAGAAGACAATTGGCAAGTATGCTAAGTCAGAATTTGAC
AAATGACCCTATAATTCTCAGATCACCTATTATATATTATGCTACATATGAAAAAAAC
TAACAGATATCATGGATAATCTCACAAAAGTTCGTGAGTATCTCAAGTCCTATTCTCG
TCTGGATCAGGCGGTAGGAGAGATAGATGAGATCGAAGCACAACGAGCTGAAAAGTC
CAATTATGAGTTGTTCCAAGAGGATGGAGTGGAAGAGCATACTAAGCCCTCTTATTTT
CAGGCAGCAGATGATTCTGACACAGAATCTGAACCAGAAATTGAAGACAATCAAGGC
TTGTATGCACCAGATCCAGAAGCTGAGCAAGTTGAAGGCTTTATACAGGGGCCTTTAG
ATGACTATGCAGATGAGGAAGTGGATGTTGTATTTACTTCGGACTGGAAACAGCCTGA
GCTTGAATCTGACGAGCATGGAAAGACCTTACGGTTGACATCGCCAGAGGGTTTAAGT
GGAGAGCAGAAATCCCAGTGGCTTTCGACGATTAAAGCAGTCGTGCAAAGTGCCAAA
TACTGGAATCTGGCAGAGTGCACATTTGAAGCATCGGGAGAAGGGGTCATTATGAAG
GAGCGCCAGATAACTCCGGATGTATATAAGGTCACTCCAGTGATGAACACACATCCGT
CCCAATCAGAAGCAGTATCAGATGTTTGGTCTCTCTCAAAGACATCCATGACTTTCCA
ACCCAAGAAAGCAAGTCTTCAGCCTCTCACCATATCCTTGGATGAATTGTTCTCATCT
AGAGGAGAGTTCATCTCTGTCGGAGGTGACGGACGAATGTCTCATAAAGAGGCCATC
CTGCTCGGCCTGAGATACAAAAAGTTGTACAATCAGGCGAGAGTCAAATATTCTCTGT
AGACTATGAAAAAAAGTAACAGATATCACGATCTAAGTGTTATCCCAATCCATTCATC
ATGAGTTCCTTAAAGAAGATTCTCGGTCTGAAGGGGAAAGGTAAGAAATCTAAGAAA
TTAGGGATCGCACCACCCCCTTATGAAGAGGACACTAGCATGGAGTATGCTCCGAGC
GCTCCAATTGACAAATCCTATTTTGGAGTTGACGAGATGGACACCTATGATCCGAATC
AATTAAGATATGAGAAATTCTTCTTTACAGTGAAAATGACGGTTAGATCTAATCGTCC
GTTCAGAACATACTCAGATGTGGCAGCCGCTGTATCCCATTGGGATCACATGTACATC
GGAATGGCAGGGAAACGTCCCTTCTACAAAATCTTGGCTTTTTTGGGTTCTTCTAATCT
AAAGGCCACTCCAGCGGTATTGGCAGATCAAGGTCAACCAGAGTATCACGCTCACTG
CGAAGGCAGGGCTTATTTGCCACATAGGATGGGGAAGACCCCTCCCATGCTCAATGTA
CCAGAGCACTTCAGAAGACCATTCAATATAGGTCTTTACAAGGGAACGATTGAGCTCA
CAATGACCATCTACGATGATGAGTCACTGGAAGCAGCTCCTATGATCTGGGATCATTT
CAATTCTTCCAAATTTTCTGATTTCAGAGAGAAGGCCTTAATGTTTGGCCTGATTGTCG
AGAAAAAGGCATCTGGAGCGTGGGTCCTGGACTCTATCGGCCACTTCAAATGAGCTA
GTCTAACTTCTAGCTTCTGAACAATCCCCGGTTTACTCAGTCTCCCCTAATTCCAGCCT
CTCGAACAACTAATATCCTGTCTTTTCTATCCCTATGAAAAAAACTAACAGAGATCGA
TCTGTTTACGCGTCTGTTCACGCGTGGTACCTTCATCATGCCGACAGAAAGCAAGAAA
GTTAGATTCGAAAATACTGCTTCAGACAAGGGGAAAAATCCTAGTAAAGTTATTAAG
AGCTACTACGGAACCATGGACATTAAGAAAATAAATGAAGGGTTATTGGACAGCAAG
ATATTAAGTGCTTTCAACACAGTGATAGCACTGCTTGGATCCATTGTAATCATAGTGA
TGAATATAATGATCATCCAAAACTACACAAGATCAACAGATAATCAGGCCATGATCA
AAGATGCATTGCAGAGTATCCAGCAGCAGATCAAGGGGCTTGCCGACAAAATTGGCA
CAGAGATAGGGCCGAAAGTATCACTGATTGATACATCCAGTACTATCACTATCCCAGC
TAATATTGGGCTGTTAGGTTCAAAGATCAGCCAGTCAACTGCAAGTATAAATGAGAAT
GTGAATGAAAAATGCAAATTTACACTGCCTCCCTTGAAAATCCACGAATGTAACATTT
CTTGTCCTAACCCACTCCCTTTTAGAGAGTATAAGCCGCAGACAGAAGGAGTGAGCAA
TCTGGTAGGATTACCTAATAATATCTGTCTGCAAAAGACATCTAATCAGATACTGAAA
CCAAAGCTGATTTCATACACCTTACCCGTAGTCGGTCAAAGTGGCACCTGTATCACAG
ACCCACTGCTGGCTATGGATGAGGGCTACTTTGCATATAGCCACCTGGAAAAAATCGG
ATCATGTTCAAGAGGGGTCTCCAAACAAAGAATAATAGGAGTTGGAGAGGTACTAGA
CAGAGGTGACGAAGTACCTTCTTTGTTTATGACTAACGTCTGGACCCCATCAAATCCA
AACACCGTTTACCATTGCAGTGCCGTGTACAACAATGAATTCTATTATGTGCTTTGTGC
AGTGTCAGTTGTTGGAGACCCTATTCTGAATAGCACCTACTGGTCCGGATCACTAATG
ATGACTCGTCTAGCTGTAAAACCTAAGAATAATGGTGAGAGTTACAATCAACATCAAT
TTGCCTTACGGAATATTGAGAAAGGGAAGTATGATAAAGTTATGCCATATGGACCCTC
AGGCATCAAACAAGGTGACACCCTGTACTTTCCTGCTGTAGGATTTTTGGTCAGGACA
GAGTTCAAATACAATGATTCAAATTGTCCCATCGCAGAGTGTCAATACAGCAAACCTG
AAAACTGCAGGCTATCTATGGGGATTAGACCAAACAGTCATTATATCCTTCGATCTGG
ACTACTAAAATACAATCTATCGGATGAGGAGAACTCTAAAATTGTATTCATTGAAATA
TCTGATCAAAGACTATCTATTGGATCTCCTAGCAAAATCTATGATTCTTTGGGTCAACC
TGTTTTCTACCAAGCATCTTTTTCATGGGACACTATGATTAAATTTGGAGATGTCCAAA
CAGTTAACCCTTTAGTTGTAAATTGGCGTGACAACACGGTAATCTCAAGACCTGGGCA
ATCACAATGCCCTAGATTCAACAAGTGCCCAGAGGTTTGCTGGGAAGGGGTTTATAAT
GATGCCTTCCTGATTGATAGAATCAATTGGATAAGCGCGGGTGTATTCCTTGACAGCA
ACCAGACCGCAGAGAATCCTGTTTTTACTGTATTCAAAGATAATGAAGTACTTTACAG
AGCACAACTAGCTTCCGAGGACACCAATGCACAAAAAACAATAACTAATTGCTTCCTT
TTGAAGAATAAGATCTGGTGTATATCACTGGTTGAGATATACGACACAGGAGACAAT
GTTATAAGACCTAAACTATTCGCAGTTAAGATACCAGAGCAATGTACATAACCTAGGC
TAGGGCTAGCCAGATTCTTCATGTTTGGACCAAATCAACTTGTGATACCATGCTCAAA
GAGGCCTCAATTATATTTGAGTTTTTAATTTTTATGAAAAAAACTAACAGCAATCATG
GAAGTCCACGATTTTGAGACCGACGAGTTCAATGATTTCAATGAAGATGACTATGCCA
CAAGAGAATTCCTGAATCCCGATGAGCGCATGACGTACTTGAATCATGCTGATTACAA
CCTGAATTCTCCTCTAATTAGTGATGATATTGACAATTTAATCAGGAAATTCAATTCTC
TTCCAATTCCCTCGATGTGGGATAGTAAGAACTGGGATGGAGTTCTTGAGATGTTAAC
ATCATGTCAAGCCAATCCCATCTCAACATCTCAGATGCATAAATGGATGGGAAGTTGG
TTAATGTCTGATAATCATGATGCCAGTCAAGGGTATAGTTTTTTACATGAAGTGGACA
AAGAGGCAGAAATAACATTTGACGTGGTGGAGACCTTCATCCGCGGCTGGGGCAACA
AACCAATTGAATACATCAAAAAGGAAAGATGGACTGACTCATTCAAAATTCTCGCTTA
TTTGTGTCAAAAGTTTTTGGACTTACACAAGTTGACATTAATCTTAAATGCTGTCTCTG
AGGTGGAATTGCTCAACTTGGCGAGGACTTTCAAAGGCAAAGTCAGAAGAAGTTCTC
ATGGAACGAACATATGCAGGATTAGGGTTCCCAGCTTGGGTCCTACTTTTATTTCAGA
AGGATGGGCTTACTTCAAGAAACTTGATATTCTAATGGACCGAAACTTTCTGTTAATG
GTCAAAGATGTGATTATAGGGAGGATGCAAACGGTGCTATCCATGGTATGTAGAATA
GACAACCTGTTCTCAGAGCAAGACATCTTCTCCCTTCTAAATATCTACAGAATTGGAG
ATAAAATTGTGGAGAGGCAGGGAAATTTTTCTTATGACTTGATTAAAATGGTGGAACC
GATATGCAACTTGAAGCTGATGAAATTAGCAAGAGAATCAAGGCCTTTAGTCCCACA
ATTCCCTCATTTTGAAAATCATATCAAGACTTCTGTTGATGAAGGGGCAAAAATTGAC
CGAGGTATAAGATTCCTCCATGATCAGATAATGAGTGTGAAAACAGTGGATCTCACAC
TGGTGATTTATGGATCGTTCAGACATTGGGGTCATCCTTTTATAGATTATTACACTGGA
CTAGAAAAATTACATTCCCAAGTAACCATGAAGAAAGATATTGATGTGTCATATGCAA
AAGCACTTGCAAGTGATTTAGCTCGGATTGTTCTATTTCAACAGTTCAATGATCATAA
AAAGTGGTTCGTGAATGGAGACTTGCTCCCTCATGATCATCCCTTTAAAAGTCATGTT
AAAGAAAATACATGGCCCACAGCTGCTCAAGTTCAAGATTTTGGAGATAAATGGCAT
GAACTTCCGCTGATTAAATGTTTTGAAATACCCGACTTACTAGACCCATCGATAATAT
ACTCTGACAAAAGTCATTCAATGAATAGGTCAGAGGTGTTGAAACATGTCCGAATGA
ATCCGAACACTCCTATCCCTAGTAAAAAGGTGTTGCAGACTATGTTGGACACAAAGGC
TACCAATTGGAAAGAATTTCTTAAAGAGATTGATGAGAAGGGCTTAGATGATGATGAT
CTAATTATTGGTCTTAAAGGAAAGGAGAGGGAACTGAAGTTGGCAGGTAGATTTTTCT
CCCTAATGTCTTGGAAATTGCGAGAATACTTTGTAATTACCGAATATTTGATAAAGAC
TCATTTCGTCCCTATGTTTAAAGGCCTGACAATGGCGGACGATCTAACTGCAGTCATT
AAAAAGATGTTAGATTCCTCATCCGGCCAAGGATTGAAGTCATATGAGGCAATTTGCA
TAGCCAATCACATTGATTACGAAAAATGGAATAACCACCAAAGGAAGTTATCAAACG
GCCCAGTGTTCCGAGTTATGGGCCAGTTCTTAGGTTATCCATCCTTAATCGAGAGAAC
TCATGAATTTTTTGAGAAAAGTCTTATATACTACAATGGAAGACCAGACTTGATGCGT
GTTCACAACAACACACTGATCAATTCAACCTCCCAACGAGTTTGTTGGCAAGGACAAG
AGGGTGGACTGGAAGGTCTACGGCAAAAAGGATGGAGTATCCTCAATCTACTGGTTA
TTCAAAGAGAGGCTAAAATCAGAAACACTGCTGTCAAAGTCTTGGCACAAGGTGATA
ATCAAGTTATTTGCACACAGTATAAAACGAAGAAATCGAGAAACGTTGTAGAATTAC
AGGGTGCTCTCAATCAAATGGTTTCTAATAATGAGAAAATTATGACTGCAATCAAAAT
AGGGACAGGGAAGTTAGGACTTTTGATAAATGACGATGAGACTATGCAATCTGCAGA
TTACTTGAATTATGGAAAAATACCGATTTTCCGTGGAGTGATTAGAGGGTTAGAGACC
AAGAGATGGTCACGAGTGACTTGTGTCACCAATGACCAAATACCCACTTGTGCTAATA
TAATGAGCTCAGTTTCCACAAATGCTCTCACCGTAGCTCATTTTGCTGAGAACCCAAT
CAATGCCATGATACAGTACAATTATTTTGGGACATTTGCTAGACTCTTGTTGATGATGC
ATGATCCTGCTCTTCGTCAATCATTGTATGAAGTTCAAGATAAGATACCGGGCTTGCA
CAGTTCTACTTTCAAATACGCCATGTTGTATTTGGACCCTTCCATTGGAGGAGTGTCGG
GCATGTCTTTGTCCAGGTTTTTGATTAGAGCCTTCCCAGATCCCGTAACAGAAAGTCTC
TCATTCTGGAGATTCATCCATGTACATGCTCGAAGTGAGCATCTGAAGGAGATGAGTG
CAGTATTTGGAAACCCCGAGATAGCCAAGTTTCGAATAACTCACATAGACAAGCTAGT
AGAAGATCCAACCTCTCTGAACATCGCTATGGGAATGAGTCCAGCGAACTTGTTAAAG
ACTGAGGTTAAAAAATGCTTAATCGAATCAAGACAAACCATCAGGAACCAGGTGATT
AAGGATGCAACCATATATTTGTATCATGAAGAGGATCGGCTCAGAAGTTTCTTATGGT
CAATAAATCCTCTGTTCCCTAGATTTTTAAGTGAATTCAAATCAGGCACTTTTTTGGGA
GTCGCAGACGGGCTCATCAGTCTATTTCAAAATTCTCGTACTATTCGGAACTCCTTTAA
GAAAAAGTATCATAGGGAATTGGATGATTTGATTGTGAGGAGTGAGGTATCCTCTTTG
ACACATTTAGGGAAACTTCATTTGAGAAGGGGATCATGTAAAATGTGGACATGTTCAG
CTACTCATGCTGACACATTAAGATACAAATCCTGGGGCCGTACAGTTATTGGGACAAC
TGTACCCCATCCATTAGAAATGTTGGGTCCACAACATCGAAAAGAGACTCCTTGTGCA
CCATGTAACACATCAGGGTTCAATTATGTTTCTGTGCATTGTCCAGACGGGATCCATG
ACGTCTTTAGTTCACGGGGACCATTGCCTGCTTATCTAGGGTCTAAAACATCTGAATCT
ACATCTATTTTGCAGCCTTGGGAAAGGGAAAGCAAAGTCCCACTGATTAAAAGAGCT
ACACGTCTTAGAGATGCTATCTCTTGGTTTGTTGAACCCGACTCTAAACTAGCAATGA
CTATACTTTCTAACATCCACTCTTTAACAGGCGAAGAATGGACCAAAAGGCAGCATGG
GTTCAAAAGAACAGGGTCTGCCCTTCATAGGTTTTCGACATCTCGGATGAGCCATGGT
GGGTTCGCATCTCAGAGCACTGCAGCATTGACCAGGTTGATGGCAACTACAGACACC
ATGAGGGATCTGGGAGATCAGAATTTCGACTTTTTATTCCAAGCAACGTTGCTCTATG
CTCAAATTACCACCACTGTTGCAAGAGACGGATGGATCACCAGTTGTACAGATCATTA
TCATATTGCCTGTAAGTCCTGTTTGAGACCCATAGAAGAGATCACCCTGGACTCAAGT
ATGGACTACACGCCCCCAGATGTATCCCATGTGCTGAAGACATGGAGGAATGGGGAA
GGTTCGTGGGGACAAGAGATAAAACAGATCTATCCTTTAGAAGGGAATTGGAAGAAT
TTAGCACCTGCTGAGCAATCCTATCAAGTCGGCAGATGTATAGGTTTTCTATATGGAG
ACTTGGCGTATAGAAAATCTACTCATGCCGAGGACAGTTCTCTATTTCCTCTATCTATA
CAAGGTCGTATTAGAGGTCGAGGTTTCTTAAAAGGGTTGCTAGACGGATTAATGAGAG
CAAGTTGCTGCCAAGTAATACACCGGAGAAGTCTGGCTCATTTGAAGAGGCCGGCCA
ACGCAGTGTACGGAGGTTTGATTTACTTGATTGATAAATTGAGTGTATCACCTCCATTC
CTTTCTCTTACTAGATCAGGACCTATTAGAGACGAATTAGAAACGATTCCCCACAAGA
TCCCAACCTCCTATCCGACAAGCAACCGTGATATGGGGGTGATTGTCAGAAATTACTT
CAAATACCAATGCCGTCTAATTGAAAAGGGAAAATACAGATCACATTATTCACAATTA
TGGTTATTCTCAGATGTCTTATCCATAGACTTCATTGGACCATTCTCTATTTCCACCAC
CCTCTTGCAAATCCTATACAAGCCATTTTTATCTGGGAAAGATAAGAATGAGTTGAGA
GAGCTGGCAAATCTTTCTTCATTGCTAAGATCAGGAGAGGGGTGGGAAGACATACAT
GTGAAATTCTTCACCAAGGACATATTATTGTGTCCAGAGGAAATCAGACATGCTTGCA
AGTTCGGGATTGCTAAGGATAATAATAAAGACATGAGCTATCCCCCTTGGGGAAGGG
AATCCAGAGGGACAATTACAACAATCCCTGTTTATTATACGACCACCCCTTACCCAAA
GATGCTAGAGATGCCTCCAAGAATCCAAAATCCCCTGCTGTCCGGAATCAGGTTGGGC
CAATTACCAACTGGCGCTCATTATAAAATTCGGAGTATATTACATGGAATGGGAATCC
ATTACAGGGACTTCTTGAGTTGTGGAGACGGCTCCGGAGGGATGACTGCTGCATTACT
ACGAGAAAATGTGCATAGCAGAGGAATATTCAATAGTCTGTTAGAATTATCAGGGTC
AGTCATGCGAGGCGCCTCTCCTGAGCCCCCCAGTGCCCTAGAAACTTTAGGAGGAGAT
AAATCGAGATGTGTAAATGGTGAAACATGTTGGGAATATCCATCTGACTTATGTGACC
CAAGGACTTGGGACTATTTCCTCCGACTCAAAGCAGGCTTGGGGCTTCAAATTGATTT
AATTGTAATGGATATGGAAGTTCGGGATTCTTCTACTAGCCTGAAAATTGAGACGAAT
GTTAGAAATTATGTGCACCGGATTTTGGATGAGCAAGGAGTTTTAATCTACAAGACTT
ATGGAACATATATTTGTGAGAGCGAAAAGAATGCAGTAACAATCCTTGGTCCCATGTT
CAAGACGGTCGACTTAGTTCAAACAGAATTTAGTAGTTCTCAAACGTCTGAAGTATAT
ATGGTATGTAAAGGTTTGAAGAAATTAATCGATGAACCCAATCCCGATTGGTCTTCCA
TCAATGAATCCTGGAAAAACCTGTACGCATTCCAGTCATCAGAACAGGAATTTGCCAG
AGCAAAGAAGGTTAGTACATACTTTACCTTGACAGGTATTCCCTCCCAATTCATTCCT
GATCCTTTTGTAAACATTGAGACTATGCTACAAATATTCGGAGTACCCACGGGTGTGT
CTCATGCGGCTGCCTTAAAATCATCTGATAGACCTGCAGATTTATTGACCATTAGCCTT
TTTTATATGGCGATTATATCGTATTATAACATCAATCATATCAGAGTAGGACCGATAC
CTCCGAACCCCCCATCAGATGGAATTGCACAAAATGTGGGGATCGCTATAACTGGTAT
AAGCTTTTGGCTGAGTTTGATGGAGAAAGACATTCCACTATATCAACAGTGTTTAGCA
GTTATCCAGCAATCATTCCCGATTAGGTGGGAGGCTGTTTCAGTAAAAGGAGGATACA
AGCAGAAGTGGAGTACTAGAGGTGATGGGCTCCCAAAAGATACCCGAATTTCAGACT
CCTTGGCCCCAATCGGGAACTGGATCAGATCTCTGGAATTGGTCCGAAACCAAGTTCG
TCTAAATCCATTCAATGAGATCTTGTTCAATCAGCTATGTCGTACAGTGGATAATCATT
TGAAATGGTCAAATTTGCGAAGAAACACAGGAATGATTGAATGGATCAATAGACGAA
TTTCAAAAGAAGACCGGTCTATACTGATGTTGAAGAGTGACCTACACGAGGAAAACT
CTTGGAGAGATTAAAAAATCATGAGGAGACTCCAAACTTTAAGTATGAAAAAAACTT
TGATCCTTAAGACCCTCTTGTGGTTTTTATTTTTTATCTGGTTTTGTGGTCTTCGTGGGT
CGGCATGGCATCTCCACCTCCTCGCGGTCCGACCTGGGCATCCGAAGGAGGACGTCGT
CCACTCGGATGGCTAAGGGAGGGGCCCCCGCGGGGCTGCTAACAAAGCCCGAAAGGA
AGCTGAGTTGGCTGCTGCCACCGCTGAGCAATAACTAGCATAACCCCTTGGGGCCTCT
AAACGGGTCTTGAGGGGTTTTTTGCTGAAAGGAGGAACTATATCCGGATCGAGACCTC
GATACTAGTGCGGTGGAGCTCCAGCTTTTGTTCCCTTTAGTGAGGGTTAATTTCGAGCT
TGGCGTAATCATGGTCATAGCTGTTTCCTGTGTGAAATTGTTATCCGCTCACAATTCCA
CACAACATACGAGCCGGAAGCATAAAGTGTAAAGCCTGGGGTGCCTAATGAGTGAGC
TAACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGAGCGG
TATCAGCTCACTCAAAGGCGGTAATACGGTTATCCACAGAATCAGGGGATAACGCAG
GAAAGAACATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAAATCGACGCT
CAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCC
TTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGG
TATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTC
ACCTAGATCCTTTTAAATTAAAAATGAAGTTTTAAATCAATCTAAAGTATATATGAGT
AAACTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATACGGG
AGGGCTTACCATCTGGCCCCAGTGCTGCAATGATACCGCGAGACCCACGCTCACCGGC
TCCAGATTTATCAGCAATAAACCAGCCAGCCGGAAGGGCCGAGCGCAGAAGTGGTCC
TGCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTGT
CACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGA
GCGGATACATATTTGAATGTATTTAGAAAAATAAACAAATAGGGGTTCCGCGCACATT
TCCCCGAAAAGTGCCACCTAAATTGTAAGCGTTAATATTTTGTTAAAATTCGCGTTAA
ATTTTTGTTAAATCAGCTCATTTTTTAACCAATAGGCCGAAATCGGCAAAATCCCTTAT
AAATCAAAAGAATAGACCGAGATAGGGTTGAGTGTTGTTCCAGTTTGGAACAAGAGT
CCACTATTAAAGAACGTGGACTCCAACGTCAAAGGGCGAAAAACCGTCTATCAGGGC
GATGGCCCACTACGTGAACCATCACCCTAATCAAGTTTTTTGGGGTCGAGGTGCCGTA
AAGCACTAAATCGGAACCCTAAAGGGAGCCCCCGATTTAGAGCTTGACGGGGAAAGC
CGGCGAACGTGGCGAGAAAGGAAGGGAAGAAAGCGAAAGGAGCGGGCGCTAGGGCG
CTGGCAAGTGTAGCGGTCACGCTGCGCGTAACCACCACACCCGCCGCGCTTAATGCGC
CGCTACAGGGCGCGTCCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGCG
ATTAAGTTGGGTAACGCCAGGGTTTTCCCAGTCACGACGTTGTAAAACGACGGCCAGT
GAATTGTAATACGACTCACTATAGG
rVSV-ΔG-NiVB G
SEQ ID NO: 2
ATCAAAATGTCTGTTACAGTCAAGAGAATCATTGACAACACAGTCGTAGTTCCAAAAC
TTCCTGCAAATGAGGATCCAGTGGAATACCCGGCAGATTACTTCAGAAAATCAAAGG
AGATTCCTCTTTACATCAATACTACAAAAAGTTTGTCAGATCTAAGAGGATATGTCTA
CCAAGGCCTCAAATCCGGAAATGTATCAATCATACATGTCAACAGCTACTTGTATGGA
GCATTAAAGGACATCCGGGGTAAGTTGGATAAAGATTGGTCAAGTTTCGGAATAAAC
ATCGGGAAAGCAGGGGATACAATCGGAATATTTGACCTTGTATCCTTGAAAGCCCTGG
ACGGCGTACTTCCAGATGGAGTATCGGATGCTTCCAGAACCAGCGCAGATGACAAAT
GGTTGCCTTTGTATCTACTTGGCTTATACAGAGTGGGCAGAACACAAATGCCTGAATA
CAGAAAAAAGCTCATGGATGGGCTGACAAATCAATGCAAAATGATCAATGAACAGTT
TGAACCTCTTGTGCCAGAAGGTCGTGACATTTTTGATGTGTGGGGAAATGACAGTAAT
TACACAAAAATTGTCGCTGCAGTGGACATGTTCTTCCACATGTTCAAAAAACATGAAT
GTGCCTCGTTCAGATACGGAACTATTGTTTCCAGATTCAAAGATTGTGCTGCATTGGC
AACATTTGGACACCTCTGCAAAATAACCGGAATGTCTACAGAAGATGTAACGACCTG
GATCTTGAACCGAGAAGTTGCAGATGAAATGGTCCAAATGATGCTTCCAGGCCAAGA
AATTGACAAGGCCGATTCATACATGCCTTATTTGATCGACTTTGGATTGTCTTCTAAGT
CTCCATATTCTTCCGTCAAAAACCCTGCCTTCCACTTCTGGGGGCAATTGACAGCTCTT
CTGCTCAGATCCACCAGAGCAAGGAATGCCCGACAGCCTGATGACATTGAGTATACA
TCTCTTACTACAGCAGGTTTGTTGTACGCTTATGCAGTAGGATCCTCTGCCGACTTGGC
ACAACAGTTTTGTGTTGGAGATAACAAATACACTCCAGATGATAGTACCGGAGGATTG
ACGACTAATGCACCGCCACAAGGCAGAGATGTGGTCGAATGGCTCGGATGGTTTGAA
GATCAAAACAGAAAACCGACTCCTGATATGATGCAGTATGCGAAAAGAGCAGTCATG
TCACTGCAAGGCCTAAGAGAGAAGACAATTGGCAAGTATGCTAAGTCAGAATTTGAC
AAATGACCCTATAATTCTCAGATCACCTATTATATATTATGCTACATATGAAAAAAAC
TAACAGATATCATGGATAATCTCACAAAAGTTCGTGAGTATCTCAAGTCCTATTCTCG
TCTGGATCAGGCGGTAGGAGAGATAGATGAGATCGAAGCACAACGAGCTGAAAAGTC
CAATTATGAGTTGTTCCAAGAGGATGGAGTGGAAGAGCATACTAAGCCCTCTTATTTT
CAGGCAGCAGATGATTCTGACACAGAATCTGAACCAGAAATTGAAGACAATCAAGGC
TTGTATGCACCAGATCCAGAAGCTGAGCAAGTTGAAGGCTTTATACAGGGGCCTTTAG
ATGACTATGCAGATGAGGAAGTGGATGTTGTATTTACTTCGGACTGGAAACAGCCTGA
GCTTGAATCTGACGAGCATGGAAAGACCTTACGGTTGACATCGCCAGAGGGTTTAAGT
GGAGAGCAGAAATCCCAGTGGCTTTCGACGATTAAAGCAGTCGTGCAAAGTGCCAAA
TACTGGAATCTGGCAGAGTGCACATTTGAAGCATCGGGAGAAGGGGTCATTATGAAG
GAGCGCCAGATAACTCCGGATGTATATAAGGTCACTCCAGTGATGAACACACATCCGT
CCCAATCAGAAGCAGTATCAGATGTTTGGTCTCTCTCAAAGACATCCATGACTTTCCA
ACCCAAGAAAGCAAGTCTTCAGCCTCTCACCATATCCTTGGATGAATTGTTCTCATCT
AGAGGAGAGTTCATCTCTGTCGGAGGTGACGGACGAATGTCTCATAAAGAGGCCATC
CTGCTCGGCCTGAGATACAAAAAGTTGTACAATCAGGCGAGAGTCAAATATTCTCTGT
AGACTATGAAAAAAAGTAACAGATATCACGATCTAAGTGTTATCCCAATCCATTCATC
ATGAGTTCCTTAAAGAAGATTCTCGGTCTGAAGGGGAAAGGTAAGAAATCTAAGAAA
TTAGGGATCGCACCACCCCCTTATGAAGAGGACACTAGCATGGAGTATGCTCCGAGC
GCTCCAATTGACAAATCCTATTTTGGAGTTGACGAGATGGACACCTATGATCCGAATC
AATTAAGATATGAGAAATTCTTCTTTACAGTGAAAATGACGGTTAGATCTAATCGTCC
GTTCAGAACATACTCAGATGTGGCAGCCGCTGTATCCCATTGGGATCACATGTACATC
GGAATGGCAGGGAAACGTCCCTTCTACAAAATCTTGGCTTTTTTGGGTTCTTCTAATCT
AAAGGCCACTCCAGCGGTATTGGCAGATCAAGGTCAACCAGAGTATCACGCTCACTG
CGAAGGCAGGGCTTATTTGCCACATAGGATGGGGAAGACCCCTCCCATGCTCAATGTA
CCAGAGCACTTCAGAAGACCATTCAATATAGGTCTTTACAAGGGAACGATTGAGCTCA
CAATGACCATCTACGATGATGAGTCACTGGAAGCAGCTCCTATGATCTGGGATCATTT
CAATTCTTCCAAATTTTCTGATTTCAGAGAGAAGGCCTTAATGTTTGGCCTGATTGTCG
AGAAAAAGGCATCTGGAGCGTGGGTCCTGGACTCTATCGGCCACTTCAAATGAGCTA
GTCTAACTTCTAGCTTCTGAACAATCCCCGGTTTACTCAGTCTCCCCTAATTCCAGCCT
CTCGAACAACTAATATCCTGTCTTTTCTATCCCTATGAAAAAAACTAACAGAGATCGA
TCTGTTTACGCGTCTGTTCACGCGTGGTACCTTCATCATGCCGACAGAAAGCAAGAAA
GTTAGATTCGAAAATACTGCTTCAGACAAGGGGAAAAATCCTAGTAAAGTTATTAAG
AGCTACTACGGAACCATGGACATTAAGAAAATAAATGAAGGGTTATTGGACAGCAAG
ATATTAAGTGCTTTCAACACAGTGATAGCACTGCTTGGATCCATTGTAATCATAGTGA
TGAATATAATGATCATCCAAAACTACACAAGATCAACAGATAATCAGGCCATGATCA
AAGATGCATTGCAGAGTATCCAGCAGCAGATCAAGGGGCTTGCCGACAAAATTGGCA
CAGAGATAGGGCCGAAAGTATCACTGATTGATACATCCAGTACTATCACTATCCCAGC
TAATATTGGGCTGTTAGGTTCAAAGATCAGCCAGTCAACTGCAAGTATAAATGAGAAT
GTGAATGAAAAATGCAAATTTACACTGCCTCCCTTGAAAATCCACGAATGTAACATTT
CTTGTCCTAACCCACTCCCTTTTAGAGAGTATAAGCCGCAGACAGAAGGAGTGAGCAA
TCTGGTAGGATTACCTAATAATATCTGTCTGCAAAAGACATCTAATCAGATACTGAAA
CCAAAGCTGATTTCATACACCTTACCCGTAGTCGGTCAAAGTGGCACCTGTATCACAG
ACCCACTGCTGGCTATGGATGAGGGCTACTTTGCATATAGCCACCTGGAAAAAATCGG
ATCATGTTCAAGAGGGGTCTCCAAACAAAGAATAATAGGAGTTGGAGAGGTACTAGA
CAGAGGTGACGAAGTACCTTCTTTGTTTATGACTAACGTCTGGACCCCATCAAATCCA
AACACCGTTTACCATTGCAGTGCCGTGTACAACAATGAATTCTATTATGTGCTTTGTGC
AGTGTCAGTTGTTGGAGACCCTATTCTGAATAGCACCTACTGGTCCGGATCACTAATG
ATGACTCGTCTAGCTGTAAAACCTAAGAATAATGGTGAGAGTTACAATCAACATCAAT
TTGCCTTACGGAATATTGAGAAAGGGAAGTATGATAAAGTTATGCCATATGGACCCTC
AGGCATCAAACAAGGTGACACCCTGTACTTTCCTGCTGTAGGATTTTTGGTCAGGACA
GAGTTCAAATACAATGATTCAAATTGTCCCATCGCAGAGTGTCAATACAGCAAACCTG
AAAACTGCAGGCTATCTATGGGGATTAGACCAAACAGTCATTATATCCTTCGATCTGG
ACTACTAAAATACAATCTATCGGATGAGGAGAACTCTAAAATTGTATTCATTGAAATA
TCTGATCAAAGACTATCTATTGGATCTCCTAGCAAAATCTATGATTCTTTGGGTCAACC
TGTTTTCTACCAAGCATCTTTTTCATGGGACACTATGATTAAATTTGGAGATGTCCAAA
CAGTTAACCCTTTAGTTGTAAATTGGCGTGACAACACGGTAATCTCAAGACCTGGGCA
ATCACAATGCCCTAGATTCAACAAGTGCCCAGAGGTTTGCTGGGAAGGGGTTTATAAT
GATGCCTTCCTGATTGATAGAATCAATTGGATAAGCGCGGGTGTATTCCTTGACAGCA
ACCAGACCGCAGAGAATCCTGTTTTTACTGTATTCAAAGATAATGAAGTACTTTACAG
AGCACAACTAGCTTCCGAGGACACCAATGCACAAAAAACAATAACTAATTGCTTCCTT
TTGAAGAATAAGATCTGGTGTATATCACTGGTTGAGATATACGACACAGGAGACAAT
GTTATAAGACCTAAACTATTCGCAGTTAAGATACCAGAGCAATGTACATAACCTAGGC
TAGGGCTAGCCAGATTCTTCATGTTTGGACCAAATCAACTTGTGATACCATGCTCAAA
GAGGCCTCAATTATATTTGAGTTTTTAATTTTTATGAAAAAAAACTAACAGCAATCAT
GGAAGTCCACGATTTTGAGACCGACGAGTTCAATGATTTCAATGAAGATGACTATGCC
ACAAGAGAATTCCTGAATCCCGATGAGCGCATGACGTACTTGAATCATGCTGATTACA
ACCTGAATTCTCCTCTAATTAGTGATGATATTGACAATTTAATCAGGAAATTCAATTCT
CTTCCAATTCCCTCGATGTGGGATAGTAAGAACTGGGATGGAGTTCTTGAGATGTTAA
CATCATGTCAAGCCAATCCCATCTCAACATCTCAGATGCATAAATGGATGGGAAGTTG
GTTAATGTCTGATAATCATGATGCCAGTCAAGGGTATAGTTTTTTACATGAAGTGGAC
AAAGAGGCAGAAATAACATTTGACGTGGTGGAGACCTTCATCCGCGGCTGGGGCAAC
AAACCAATTGAATACATCAAAAAGGAAAGATGGACTGACTCATTCAAAATTCTCGCTT
ATTTGTGTCAAAAGTTTTTGGACTTACACAAGTTGACATTAATCTTAAATGCTGTCTCT
GAGGTGGAATTGCTCAACTTGGCGAGGACTTTCAAAGGCAAAGTCAGAAGAAGTTCT
CATGGAACGAACATATGCAGGATTAGGGTTCCCAGCTTGGGTCCTACTTTTATTTCAG
AAGGATGGGCTTACTTCAAGAAACTTGATATTCTAATGGACCGAAACTTTCTGTTAAT
GGTCAAAGATGTGATTATAGGGAGGATGCAAACGGTGCTATCCATGGTATGTAGAAT
AGACAACCTGTTCTCAGAGCAAGACATCTTCTCCCTTCTAAATATCTACAGAATTGGA
GATAAAATTGTGGAGAGGCAGGGAAATTTTTCTTATGACTTGATTAAAATGGTGGAAC
CGATATGCAACTTGAAGCTGATGAAATTAGCAAGAGAATCAAGGCCTTTAGTCCCAC
AATTCCCTCATTTTGAAAATCATATCAAGACTTCTGTTGATGAAGGGGCAAAAATTGA
CCGAGGTATAAGATTCCTCCATGATCAGATAATGAGTGTGAAAACAGTGGATCTCACA
CTGGTGATTTATGGATCGTTCAGACATTGGGGTCATCCTTTTATAGATTATTACACTGG
ACTAGAAAAATTACATTCCCAAGTAACCATGAAGAAAGATATTGATGTGTCATATGCA
AAAGCACTTGCAAGTGATTTAGCTCGGATTGTTCTATTTCAACAGTTCAATGATCATA
AAAAGTGGTTCGTGAATGGAGACTTGCTCCCTCATGATCATCCCTTTAAAAGTCATGT
TAAAGAAAATACATGGCCCACAGCTGCTCAAGTTCAAGATTTTGGAGATAAATGGCA
TGAACTTCCGCTGATTAAATGTTTTGAAATACCCGACTTACTAGACCCATCGATAATA
TACTCTGACAAAAGTCATTCAATGAATAGGTCAGAGGTGTTGAAACATGTCCGAATGA
ATCCGAACACTCCTATCCCTAGTAAAAAGGTGTTGCAGACTATGTTGGACACAAAGGC
TACCAATTGGAAAGAATTTCTTAAAGAGATTGATGAGAAGGGCTTAGATGATGATGAT
CTAATTATTGGTCTTAAAGGAAAGGAGAGGGAACTGAAGTTGGCAGGTAGATTTTTCT
CCCTAATGTCTTGGAAATTGCGAGAATACTTTGTAATTACCGAATATTTGATAAAGAC
TCATTTCGTCCCTATGTTTAAAGGCCTGACAATGGCGGACGATCTAACTGCAGTCATT
AAAAAGATGTTAGATTCCTCATCCGGCCAAGGATTGAAGTCATATGAGGCAATTTGCA
TAGCCAATCACATTGATTACGAAAAATGGAATAACCACCAAAGGAAGTTATCAAACG
GCCCAGTGTTCCGAGTTATGGGCCAGTTCTTAGGTTATCCATCCTTAATCGAGAGAAC
TCATGAATTTTTTGAGAAAAGTCTTATATACTACAATGGAAGACCAGACTTGATGCGT
GTTCACAACAACACACTGATCAATTCAACCTCCCAACGAGTTTGTTGGCAAGGACAAG
AGGGTGGACTGGAAGGTCTACGGCAAAAAGGATGGAGTATCCTCAATCTACTGGTTA
TTCAAAGAGAGGCTAAAATCAGAAACACTGCTGTCAAAGTCTTGGCACAAGGTGATA
ATCAAGTTATTTGCACACAGTATAAAACGAAGAAATCGAGAAACGTTGTAGAATTAC
AGGGTGCTCTCAATCAAATGGTTTCTAATAATGAGAAAATTATGACTGCAATCAAAAT
AGGGACAGGGAAGTTAGGACTTTTGATAAATGACGATGAGACTATGCAATCTGCAGA
TTACTTGAATTATGGAAAAATACCGATTTTCCGTGGAGTGATTAGAGGGTTAGAGACC
AAGAGATGGTCACGAGTGACTTGTGTCACCAATGACCAAATACCCACTTGTGCTAATA
TAATGAGCTCAGTTTCCACAAATGCTCTCACCGTAGCTCATTTTGCTGAGAACCCAAT
CAATGCCATGATACAGTACAATTATTTTGGGACATTTGCTAGACTCTTGTTGATGATGC
ATGATCCTGCTCTTCGTCAATCATTGTATGAAGTTCAAGATAAGATACCGGGCTTGCA
CAGTTCTACTTTCAAATACGCCATGTTGTATTTGGACCCTTCCATTGGAGGAGTGTCGG
GCATGTCTTTGTCCAGGTTTTTGATTAGAGCCTTCCCAGATCCCGTAACAGAAAGTCTC
TCATTCTGGAGATTCATCCATGTACATGCTCGAAGTGAGCATCTGAAGGAGATGAGTG
CAGTATTTGGAAACCCCGAGATAGCCAAGTTTCGAATAACTCACATAGACAAGCTAGT
AGAAGATCCAACCTCTCTGAACATCGCTATGGGAATGAGTCCAGCGAACTTGTTAAAG
ACTGAGGTTAAAAAATGCTTAATCGAATCAAGACAAACCATCAGGAACCAGGTGATT
AAGGATGCAACCATATATTTGTATCATGAAGAGGATCGGCTCAGAAGTTTCTTATGGT
CAATAAATCCTCTGTTCCCTAGATTTTTAAGTGAATTCAAATCAGGCACTTTTTTGGGA
GTCGCAGACGGGCTCATCAGTCTATTTCAAAATTCTCGTACTATTCGGAACTCCTTTAA
GAAAAAGTATCATAGGGAATTGGATGATTTGATTGTGAGGAGTGAGGTATCCTCTTTG
ACACATTTAGGGAAACTTCATTTGAGAAGGGGATCATGTAAAATGTGGACATGTTCAG
CTACTCATGCTGACACATTAAGATACAAATCCTGGGGCCGTACAGTTATTGGGACAAC
TGTACCCCATCCATTAGAAATGTTGGGTCCACAACATCGAAAAGAGACTCCTTGTGCA
CCATGTAACACATCAGGGTTCAATTATGTTTCTGTGCATTGTCCAGACGGGATCCATG
ACGTCTTTAGTTCACGGGGACCATTGCCTGCTTATCTAGGGTCTAAAACATCTGAATCT
ACATCTATTTTGCAGCCTTGGGAAAGGGAAAGCAAAGTCCCACTGATTAAAAGAGCT
ACACGTCTTAGAGATGCTATCTCTTGGTTTGTTGAACCCGACTCTAAACTAGCAATGA
CTATACTTTCTAACATCCACTCTTTAACAGGCGAAGAATGGACCAAAAGGCAGCATGG
GTTCAAAAGAACAGGGTCTGCCCTTCATAGGTTTTCGACATCTCGGATGAGCCATGGT
GGGTTCGCATCTCAGAGCACTGCAGCATTGACCAGGTTGATGGCAACTACAGACACC
ATGAGGGATCTGGGAGATCAGAATTTCGACTTTTTATTCCAAGCAACGTTGCTCTATG
CTCAAATTACCACCACTGTTGCAAGAGACGGATGGATCACCAGTTGTACAGATCATTA
TCATATTGCCTGTAAGTCCTGTTTGAGACCCATAGAAGAGATCACCCTGGACTCAAGT
ATGGACTACACGCCCCCAGATGTATCCCATGTGCTGAAGACATGGAGGAATGGGGAA
GGTTCGTGGGGACAAGAGATAAAACAGATCTATCCTTTAGAAGGGAATTGGAAGAAT
TTAGCACCTGCTGAGCAATCCTATCAAGTCGGCAGATGTATAGGTTTTCTATATGGAG
ACTTGGCGTATAGAAAATCTACTCATGCCGAGGACAGTTCTCTATTTCCTCTATCTATA
CAAGGTCGTATTAGAGGTCGAGGTTTCTTAAAAGGGTTGCTAGACGGATTAATGAGAG
CAAGTTGCTGCCAAGTAATACACCGGAGAAGTCTGGCTCATTTGAAGAGGCCGGCCA
ACGCAGTGTACGGAGGTTTGATTTACTTGATTGATAAATTGAGTGTATCACCTCCATTC
CTTTCTCTTACTAGATCAGGACCTATTAGAGACGAATTAGAAACGATTCCCCACAAGA
TCCCAACCTCCTATCCGACAAGCAACCGTGATATGGGGGTGATTGTCAGAAATTACTT
CAAATACCAATGCCGTCTAATTGAAAAGGGAAAATACAGATCACATTATTCACAATTA
TGGTTATTCTCAGATGTCTTATCCATAGACTTCATTGGACCATTCTCTATTTCCACCAC
CCTCTTGCAAATCCTATACAAGCCATTTTTATCTGGGAAAGATAAGAATGAGTTGAGA
GAGCTGGCAAATCTTTCTTCATTGCTAAGATCAGGAGAGGGGTGGGAAGACATACAT
GTGAAATTCTTCACCAAGGACATATTATTGTGTCCAGAGGAAATCAGACATGCTTGCA
AGTTCGGGATTGCTAAGGATAATAATAAAGACATGAGCTATCCCCCTTGGGGAAGGG
AATCCAGAGGGACAATTACAACAATCCCTGTTTATTATACGACCACCCCTTACCCAAA
GATGCTAGAGATGCCTCCAAGAATCCAAAATCCCCTGCTGTCCGGAATCAGGTTGGGC
CAATTACCAACTGGCGCTCATTATAAAATTCGGAGTATATTACATGGAATGGGAATCC
ATTACAGGGACTTCTTGAGTTGTGGAGACGGCTCCGGAGGGATGACTGCTGCATTACT
ACGAGAAAATGTGCATAGCAGAGGAATATTCAATAGTCTGTTAGAATTATCAGGGTC
AGTCATGCGAGGCGCCTCTCCTGAGCCCCCCAGTGCCCTAGAAACTTTAGGAGGAGAT
AAATCGAGATGTGTAAATGGTGAAACATGTTGGGAATATCCATCTGACTTATGTGACC
CAAGGACTTGGGACTATTTCCTCCGACTCAAAGCAGGCTTGGGGCTTCAAATTGATTT
AATTGTAATGGATATGGAAGTTCGGGATTCTTCTACTAGCCTGAAAATTGAGACGAAT
GTTAGAAATTATGTGCACCGGATTTTGGATGAGCAAGGAGTTTTAATCTACAAGACTT
ATGGAACATATATTTGTGAGAGCGAAAAGAATGCAGTAACAATCCTTGGTCCCATGTT
CAAGACGGTCGACTTAGTTCAAACAGAATTTAGTAGTTCTCAAACGTCTGAAGTATAT
ATGGTATGTAAAGGTTTGAAGAAATTAATCGATGAACCCAATCCCGATTGGTCTTCCA
TCAATGAATCCTGGAAAAACCTGTACGCATTCCAGTCATCAGAACAGGAATTTGCCAG
AGCAAAGAAGGTTAGTACATACTTTACCTTGACAGGTATTCCCTCCCAATTCATTCCT
GATCCTTTTGTAAACATTGAGACTATGCTACAAATATTCGGAGTACCCACGGGTGTGT
CTCATGCGGCTGCCTTAAAATCATCTGATAGACCTGCAGATTTATTGACCATTAGCCTT
TTTTATATGGCGATTATATCGTATTATAACATCAATCATATCAGAGTAGGACCGATAC
CTCCGAACCCCCCATCAGATGGAATTGCACAAAATGTGGGGATCGCTATAACTGGTAT
AAGCTTTTGGCTGAGTTTGATGGAGAAAGACATTCCACTATATCAACAGTGTTTAGCA
GTTATCCAGCAATCATTCCCGATTAGGTGGGAGGCTGTTTCAGTAAAAGGAGGATACA
AGCAGAAGTGGAGTACTAGAGGTGATGGGCTCCCAAAAGATACCCGAATTTCAGACT
CCTTGGCCCCAATCGGGAACTGGATCAGATCTCTGGAATTGGTCCGAAACCAAGTTCG
TCTAAATCCATTCAATGAGATCTTGTTCAATCAGCTATGTCGTACAGTGGATAATCATT
TGAAATGGTCAAATTTGCGAAGAAACACAGGAATGATTGAATGGATCAATAGACGAA
TTTCAAAAGAAGACCGGTCTATACTGATGTTGAAGAGTGACCTACACGAGGAAAACT
CTTGGAGAGATTAAAAAATCATGAGGAGACTCCAAACTTTAAGTATGAAAAAAACTT
TGATCCTTAAGACCCTCTTGTGGTTTTTATTTTTTATCTGGTTTTGTGGTCTTCGT
VSV N protein sequence
SEQ ID NO: 3
MSVTVKRIIDNTVVVPKLPANEDPVEYPADYFRKSKEIPLYINTTKSLSDLRGYVYQGLKS
GNVSIIHVNSYLYGALKDIRGKLDKDWSSFGINIGKAGDTIGIFDLVSLKALDGVLPDGVS
DASRTSADDKWLPLYLLGLYRVGRTQMPEYRKKLMDGLTNQCKMINEQFEPLVPEGRDI
FDVWGNDSNYTKIVAAVDMFFHMFKKHECASFRYGTIVSRFKDCAALATFGHLCKITGM
STEDVTTWILNREVADEMVQMMLPGQEIDKADSYMPYLIDFGLSSKSPYSSVKNPAFHFW
GQLTALLLRSTRARNARQPDDIEYTSLTTAGLLYAYAVGSSADLAQQFCVGDNKYTPDDS
TGGLTTNAPPQGRDVVEWLGWFEDQNRKPTPDMMQYAKRAVMSLQGLREKTIGKYAKS
EFDK*
VSV P protein sequence
SEQ ID NO: 4
MDNLTKVREYLKSYSRLDQAVGEIDEIEAQRAEKSNYELFQEDGVEEHTKPSYFQAADDS
DTESEPEIEDNQGLYAPDPEAEQVEGFIQGPLDDYADEEVDVVFTSDWKQPELESDEHGK
TLRLTSPEGLSGEQKSQWLSTIKAVVQSAKYWNLAECTFEASGEGVIMKERQITPDVYKV
TPVMNTHPSQSEAVSDVWSLSKTSMTFQPKKASLQPLTISLDELFSSRGEFISVGGDGRMS
HKEAILLGLRYKKLYNQARVKYSL*
VSV M protein sequence
SEQ ID NO: 5
MSSLKKILGLKGKGKKSKKLGIAPPPYEEDTSMEYAPSAPIDKSYFGVDEMDTYDPNQLR
YEKFFFTVKMTVRSNRPFRTYSDVAAAVSHWDHMYIGMAGKRPFYKILAFLGSSNLKAT
PAVLADQGQPEYHAHCEGRAYLPHRMGKTPPMLNVPEHFRRPFNIGLYKGTIELTMTIYD
DESLEAAPMIWDHFNSSKFSDFREKALMFGLIVEKKASGAWVLDSIGHFK*
NiV_{B} G protein sequence
SEQ ID NO: 6
MPTESKKVRFENTASDKGKNPSKVIKSYYGTMDIKKINEGLLDSKILSAFNTVIALLGSIVII
VMNIMIIQNYTRSTDNQAMIKDALQSIQQQIKGLADKIGTEIGPKVSLIDTSSTITIPANIGLL
GSKISQSTASINENVNEKCKFTLPPLKIHECNISCPNPLPFREYKPQTEGVSNLVGLPNNICL
QKTSNQILKPKLISYTLPVVGQSGTCITDPLLAMDEGYFAYSHLEKIGSCSRGVSKQRIIGV
GEVLDRGDEVPSLFMTNVWTPSNPNTVYHCSAVYNNEFYYVLCAVSVVGDPILNSTYWS
GSLMMTRLAVKPKNNGESYNQHQFALRNIEKGKYDKVMPYGPSGIKQGDTLYFPAVGFL
VRTEFKYNDSNCPIAECQYSKPENCRLSMGIRPNSHYILRSGLLKYNLSDEENSKIVFIEISD
QRLSIGSPSKIYDSLGQPVFYQASFSWDTMIKFGDVQTVNPLVVNWRDNTVISRPGQSQCP
RFNKCPEVCWEGVYNDAFLIDRINWISAGVFLDSNQTAENPVFTVFKDNEVLYRAQLASE
DTNAQKTITNCFLLKNKIWCISLVEIYDTGDNVIRPKLFAVKIPEQCT*
VSV L protein sequence
SEQ ID NO: 7
MEVHDFETDEFNDFNEDDYATREFLNPDERMTYLNHADYNLNSPLISDDIDNLIRKENSLP
IPSMWDSKNWDGVLEMLTSCQANPISTSQMHKWMGSWLMSDNHDASQGYSFLHEVDK
EAEITFDVVETFIRGWGNKPIEYIKKERWTDSFKILAYLCQKFLDLHKLTLILNAVSEVELL
NLARTFKGKVRRSSHGTNICRIRVPSLGPTFISEGWAYFKKLDILMDRNFLLMVKDVIIGR
MQTVLSMVCRIDNLFSEQDIFSLLNIYRIGDKIVERQGNFSYDLIKMVEPICNLKLMKLARE
SRPLVPQFPHFENHIKTSVDEGAKIDRGIRFLHDQIMSVKTVDLTLVIYGSFRHWGHPFIDY
YTGLEKLHSQVTMKKDIDVSYAKALASDLARIVLFQQFNDHKKWFVNGDLLPHDHPFKS
HVKENTWPTAAQVQDFGDKWHELPLIKCFEIPDLLDPSIIYSDKSHSMNRSEVLKHVRMN
PNTPIPSKKVLQTMLDTKATNWKEFLKEIDEKGLDDDDLIIGLKGKERELKLAGRFFSLMS
WKLREYFVITEYLIKTHFVPMFKGLTMADDLTAVIKKMLDSSSGQGLKSYEAICIANHIDY
EKWNNHQRKLSNGPVFRVMGQFLGYPSLIERTHEFFEKSLIYYNGRPDLMRVHNNTLINS
TSQRVCWQGQEGGLEGLRQKGWSILNLLVIQREAKIRNTAVKVLAQGDNQVICTQYKTK
KSRNVVELQGALNQMVSNNEKIMTAIKIGTGKLGLLINDDETMQSADYLNYGKIPIFRGVI
RGLETKRWSRVTCVTNDQIPTCANIMSSVSTNALTVAHFAENPINAMIQYNYFGTFARLLL
MMHDPALRQSLYEVQDKIPGLHSSTFKYAMLYLDPSIGGVSGMSLSRFLIRAFPDPVTESL
SFWRFIHVHARSEHLKEMSAVFGNPEIAKFRITHIDKLVEDPTSLNIAMGMSPANLLKTEV
KKCLIESRQTIRNQVIKDATIYLYHEEDRLRSFLWSINPLFPRFLSEFKSGTFLGVADGLISLF
QNSRTIRNSFKKKYHRELDDLIVRSEVSSLTHLGKLHLRRGSCKMWTCSATHADTLRYKS
WGRTVIGTTVPHPLEMLGPQHRKETPCAPCNTSGFNYVSVHCPDGIHDVFSSRGPLPAYL
GSKTSESTSILQPWERESKVPLIKRATRLRDAISWFVEPDSKLAMTILSNIHSLTGEEWTKR
QHGFKRTGSALHRFSTSRMSHGGFASQSTAALTRLMATTDTMRDLGDQNFDFLFQATLL
YAQITTTVARDGWITSCTDHYHIACKSCLRPIEEITLDSSMDYTPPDVSHVLKTWRNGEGS
WGQEIKQIYPLEGNWKNLAPAEQSYQVGRCIGFLYGDLAYRKSTHAEDSSLFPLSIQGRIR
GRGFLKGLLDGLMRASCCQVIHRRSLAHLKRPANAVYGGLIYLIDKLSVSPPFLSLTRSGPI
RDELETIPHKIPTSYPTSNRDMGVIVRNYFKYQCRLIEKGKYRSHYSQLWLFSDVLSIDFIG
PFSISTTLLQILYKPFLSGKDKNELRELANLSSLLRSGEGWEDIHVKFFTKDILLCPEEIRHA
CKFGIAKDNNKDMSYPPWGRESRGTITTIPVYYTTTPYPKMLEMPPRIQNPLLSGIRLGQLP
TGAHYKIRSILHGMGIHYRDFLSCGDGSGGMTAALLRENVHSRGIFNSLLELSGSVMRGA
SPEPPSALETLGGDKSRCVNGETCWEYPSDLCDPRTWDYFLRLKAGLGLQIDLIVMDMEV
RDSSTSLKIETNVRNYVHRILDEQGVLIYKTYGTYICESEKNAVTILGPMFKTVDLVQTEFS
SSQTSEVYMVCKGLKKLIDEPNPDWSSINESWKNLYAFQSSEQEFARAKKVSTYFTLTGIP
SQFIPDPFVNIETMLQIFGVPTGVSHAAALKSSDRPADLLTISLFYMAIISYYNINHIRVGPIP
PNPPSDGIAQNVGIAITGISFWLSLMEKDIPLYQQCLAVIQQSFPIRWEAVSVKGGYKQKW
STRGDGLPKDTRISDSLAPIGNWIRSLELVRNQVRLNPFNEILFNQLCRTVDNHLKWSNLR
RNTGMIEWINRRISKEDRSILMLKSDLHEENSWRD*

### Example 2. A Recombinant Vesicular Stomatitis Virus-Based Vaccine Fully Protects Nonhuman Primates Against Nipah Virus Disease One Year after Immunization

No licensed countermeasures exist against Nipah virus (NiV), which causes a frequently fatal respiratory disease and encephalitis in humans. An ideal vaccine would confer both fast-acting and long-lived protection. Example 1 shows the generation of a recombinant vesicular stomatitis virus (rVSV)-based vaccine expressing the NiV glycoprotein (rVSV-ΔG-NiV_{B} G) that protected 100% of nonhuman primates from NiV-associated lethality within a week. Here, to evaluate the durability of rVSV-ΔG-NiV_{B} G, the inventors vaccinated African green monkeys (AGMs) one year prior to challenge with a uniformly lethal dose of NiV. The rVSV-ΔG-NiV_{B} G vaccine induced stable and robust humoral responses, whereas cellular responses generated were modest. All immunized AGMs (whether receiving a single dose or prime-boosted) survived with no detectable clinical signs or NiV replication. Transcriptomic analyses indicated adaptive immune signatures correlated with vaccine-mediated protection. While vaccines for certain respiratory infections (e.g., COVID-19) have yet to provide durable protection, these results demonstrate that the rVSV-ΔG-NiV_{B} G elicits long-lasting immunity.

Fast acting and durable vaccines are desperately needed to combat NiV outbreaks. Several vaccines have shown promise in preclinical models, but no licensed vaccines or therapeutics are available for human use. Most vaccine candidates target the NiV surface glycoprotein (G) and/or fusion protein (F) as immunogens as these proteins are required for virus entry and are readily recognized by the host immune system⁷. For example, vaccinia and canarypox vectors encoding NiV_{M}F or NiV_{M}G have shown protection against NiV_{M} in hamsters and pigs^{8, 9}; a recombinant chimpanzee adenovirus vaccine expressing NiV_{B} G completely protected hamsters against exposure to NiV_{B} and NiV_{M}¹⁰; and a recombinant adenovirus-associated virus vaccine expressing NiV_{M}G completely protected hamsters against a homologous NiV_{M} challenge¹¹. Other vaccine candidates include a virus-like particle-based NiV vaccine that protected hamsters against a homologous NiV_{M} challenge¹² and a messenger ribonucleic acid (mRNA) vaccine encoding HeV G that protected 70% of hamsters and reduced viral load against a NiV_{M} challenge¹³. The latter vaccine recently advanced to Phase I clinical trials in humans (Clinical Trial #NCT05398796), although its efficacy in the most stringent animal model, NHPs, has not yet been reported. As vaccine and therapeutic protection is generally more difficult to achieve in NHP versus rodent models, a demonstration of countermeasure efficacy in the NHP model is ideal. NHP testing is also useful for immunobridging purposes in the absence of human efficacy data.

African green monkeys (AGMs) are considered the “gold standard” NHP model for NiV as they most accurately mimic human disease¹⁴. Some promising vaccines include a recombinant measles virus vector expressing NiV_{M} G that demonstrated partial efficacy in AGMs against NiV_{M}¹⁵ and a Hendra virus G subunit vaccine (HeV sG) that fully defended AGMs against NiV_{M}- and NiV_{B}-associated lethality^{16, 17} Alum-adjuvanted HeV sG is currently in phase 1 trials to assess its safety profile in humans¹⁸ (Clinical Trial #NCT04199169).

Most vaccine candidates require multiple injections to confer protective efficacy, yet a single dose of rVSV-ΔG-NiV_{B} G one week prior to NiV exposure protected 100% of AGMs from lethality. At three days post immunization, 66% of AGMs were protected, demonstrating the rapid immunostimulatory properties of the vaccine. Ervebo was found to decrease transmission to close contacts of infected individuals in a Phase III ring vaccination trial in Guinea during the 2013-2016 West Africa and 2018-2020 DRC Ebola virus outbreaks^{26, 27, 28}. Similarly, a reactive vaccination approach with rVSV-ΔG-NiV_{B} G could help contain NiV outbreaks.

For outbreak management or a deliberate release, the need for multiple injections of a vaccine over a prolonged period is neither practical nor economical. A single dose vaccine providing long-lived protection is ideal to prevent future occurrences and flare-ups of NiV disease. To test the ability of rVSV-ΔG-NiV_{B} G to elicit sustained immunity, the inventors vaccinated AGMs one year prior to challenge with a uniformly lethal dose of NiV. Humoral and cellular responses were monitored over the course of the study. An assessment of durability is crucial for evaluating the suitability of any vaccine destined for clinical use.

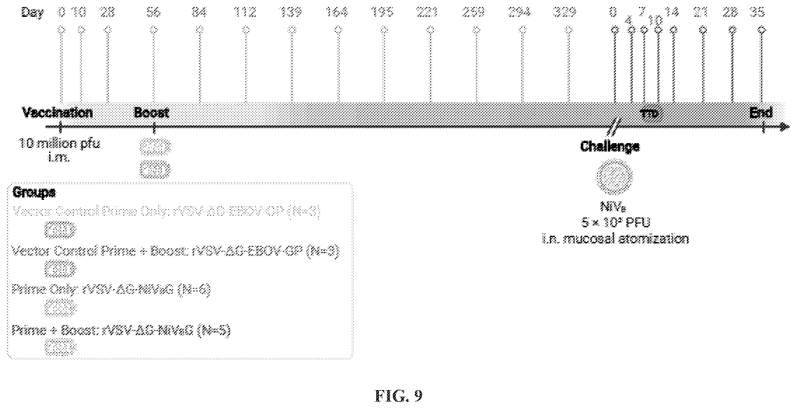

Study design and vaccination. As shown in the example above, the generation of a single cycle rVSV vaccine that elicited rapid protection against NiV disease²⁵ (FIG. 9). To evaluate the durability of rVSV-ΔG-NiV_{B} G, the inventors randomized seventeen healthy adult AGMs into four groups: prime only (N=6), prime+boost (N=5), vector control prime (N=3), and vector control prime+boost (N=3) cohorts (FIG. 9). AGMs were intramuscularly (i.m.) vaccinated with a 1×10⁷ PFU dose of rVSV-ΔG-NiV_{B} G or a non-specific rVSV vector control expressing the Ebola virus glycoprotein (rVSV-ΔG-EBOV-GP). The prime+boost and vector control prime+boost groups received an additional dose of each respective vaccine at 56 days post vaccination (i.e., 28 days after prime immunization). Blood samples were collected at least monthly on days 0, 10, 28, 56, 84, 112, 139, 164, 195, 221, 259, 294, 329, and prior to challenge to evaluate humoral and cellular responses over time. Approximately one year after administration of the first dose (369 days), all AGMs were challenged intranasally (i.n.) with a uniformly lethal dose of 5×10³ PFU of NiV_{B} as previously described²⁹. Post-exposure blood samples were collected at 4, 7, 10, 14, 21, 28, terminally, and/or 35 days.

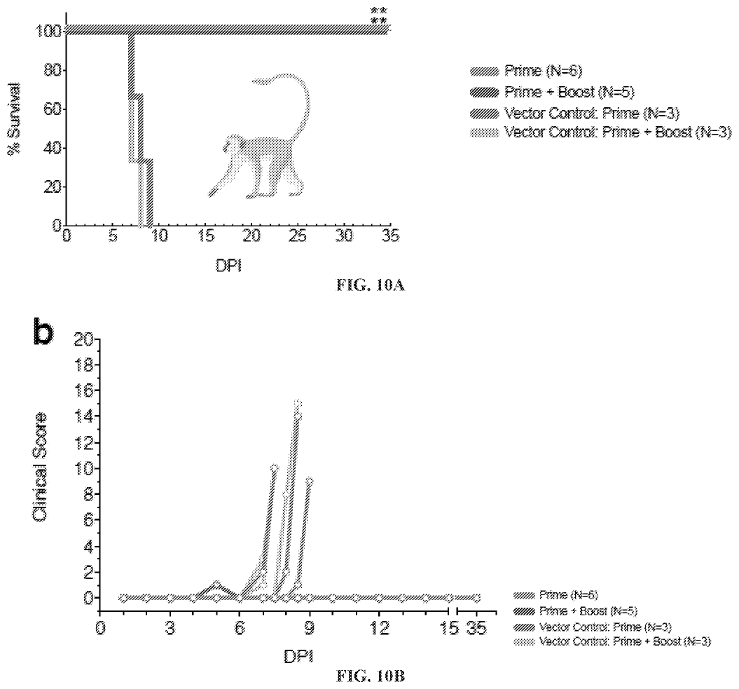

Survival and clinical signs. Whether receiving a single dose of vaccine or prime-boosted, 100% of specifically immunized AGM subjects survived to the 35-day post infection (DPI) study endpoint (FIG. 10A). A statistically significant association was observed between prime and vector control prime groups (log-rank p=0.0018) and prime+boost and vector control prime+boost groups (log-rank p=0.0046), but not between prime and prime+boost groups. AGMs receiving a single or two doses of the nonspecific rVSV vaccine reached euthanasia criteria by 7-9 DPI. No clinical signs were evident in prime or prime+boosted groups (FIG. 10B) other than transient anorexia in subject PB-2 at 5 DPI. Respiration rates did not significantly fluctuate from baseline levels in the groups vaccinated with rVSV-ΔG-NiV_{B} G, whereas the vector control groups developed tachypnea and dyspnea indicating respiratory decline (FIG. 10C).

Various hematological and serum biochemistry changes occurred throughout the vaccination and challenge phases of the study in all cohorts. Clinical pathology in prime and prime+boost groups was mild, but some subjects exhibited decreased appetite as well as transient increases in alanine transferase (ALT), aspartate transaminase (AST), or gamma-glutamyl transferase (GGT)(data not shown). After NiV_{B} exposure, all subjects in the vector control groups had elevated C-reactive protein (CRP) values, indicating non-specific systemic inflammation. All controls except VC-P2 exhibited lymphocytopenia and 4/6 subjects (VC-P-2, VC-P-3, VC-PB-1, VC-PB-2) developed thrombocytopenia. Monocytosis, neutrophilia, and hypoamylasemia were also prominent findings in these vector controls.

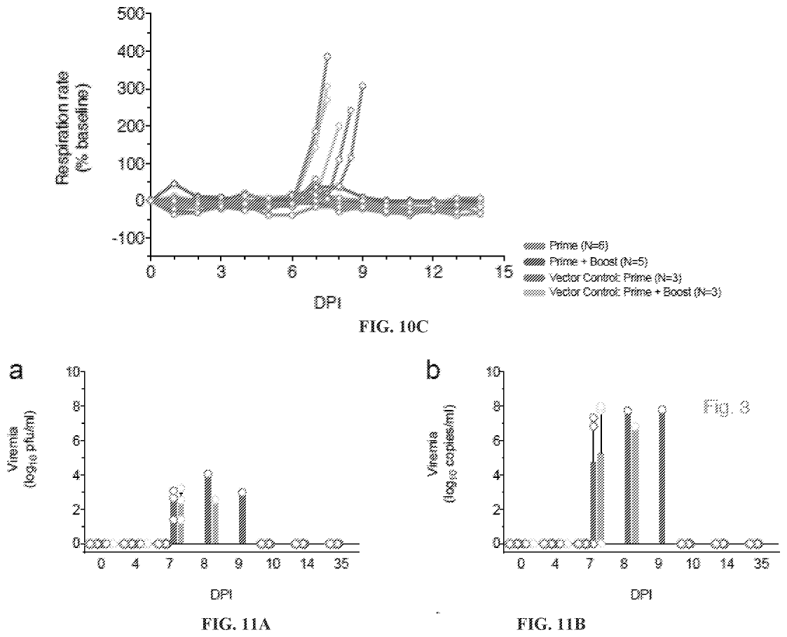

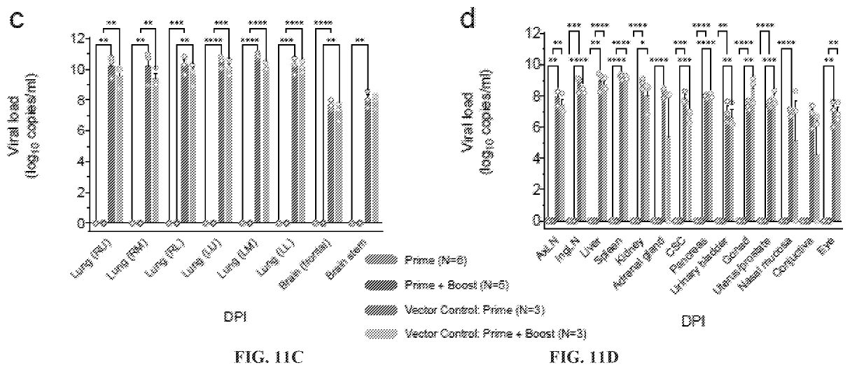

Viral loads in vaccinated AGMs challenged with NiV_{B}. Viremia was undetectable by plaque assay or qRT-PCR in subjects receiving a single or two doses of rVSV-ΔG-NiV_{B} G (FIGS. 11A, 11B). All vector controls, whether primed only or prime-boosted, developed viremia following challenge. Shortly prior to euthanasia, infectious NiV_{B} titers in control subjects ranged from 2 to 4 log₁₀ pfu/ml while viral copies ranged from 6 to 8 log₁₀ genome equivalents (GEq) per ml.

Similarly, NiV_{B} replication in lung and neurological tissue (FIG. 11C) or any other major organs (FIG. 11D) was undetectable in all rVSV-ΔG-NiV_{B} G-immunized subjects. Thus, specific vaccination induced sterilizing immunity. High viral loads were found in all organs tested for the vector control groups including lymph node, liver, spleen, kidney, urinary, reproductive, and mucosal tissue (FIG. 11C, 11D).

Pathology of vaccinated AGMs challenged with NiV_{B}. Necropsy was performed on AGMs after euthanasia. Lesions consistent with NiV disease were present in all vector controls (VC-P-1, VC-P-2, VC-P-3, VC-PB-1, VC-PB-2, VC-PB-3) including hemorrhagic pneumonia with extensive pleura effusion, hepatic congestion, splenomegaly, and meningeal congestion. Other lesions included adrenomegaly (VC-P-1, VC-P-2, VC-P-3, VC-PB-1, VC-PB-2, VC-PB-3) and lymphadenomegaly (VC-P-2). No gross lesions were apparent in specifically vaccinated subjects.

Prominent histological findings (FIGS. 12A-12R) in vector control examined sections included interstitial pneumonia (characterized by the expansion of alveolar septa with mixed mononuclear inflammation, edema, hemorrhage and in some areas, necrotic cellular debris) and flooded alveolar spaces (with numerous mononuclear cells, edema, hemorrhage, and occasionally necrotic debris merged imperceptibly with the remnants of the alveolar septa) (FIG. 12A). Immunohistochemistry (IHC) positivity was prevalent in the endothelium lining alveolar septa, the endothelium of medium caliber vessels, mononuclear cells within the alveolar septa, alveolar macrophages, and rarely the smooth muscle of large caliber vessels (FIG. 12B). Loss of typical germinal center architecture of the spleen was evident in vector control sections with reduced lymphocyte population, flooding of white pulp with hemorrhage, and the presence of multinucleated cells (FIG. 12E). Within the white pulp and scattered throughout the red pulp of the spleen, antigen reactivity was concentrated within mononuclear cells and syncytial cells (FIG. 12F). Other common lesions in vector controls included lymphoid medullary histiocytosis (axillary and inguinal), sinusoidal leukocytosis of the liver (FIG. 12I), nephritis (FIG. 12M), adrenalitis, mild gliosis of the brain (FIG. 12Q), esophagitis, tracheitis, myocarditis, cystitis, and rarely prostatitis. In these examined sections, anti-NiV immunolabeling was predominantly in mononuclear inflammatory cells and the endothelium. At the study end point, no histologic or immunolabeling was present in corresponding tissues from rVSV-ΔG-NiV_{B} G-primed or prime-boosted subjects (FIG. 12C, 12D, 12G, 12H, 12K, 12L, 12O, 12P, 12S, 12T) or any other examined tissue: cervical spinal cord, pituitary gland, trigeminal ganglion, brain (frontal), brainstem, hippocampus, trachea/esophagus, heart, pancreas, urinary bladder, gonad, uterus/prostate, conjunctiva, nasal tissue, and eye.

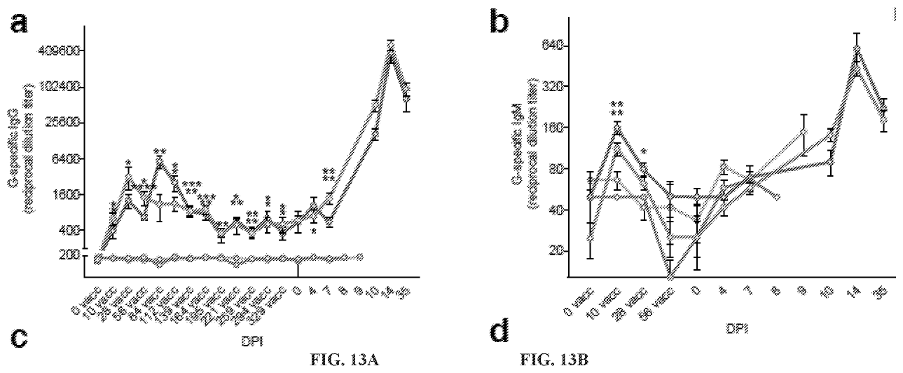

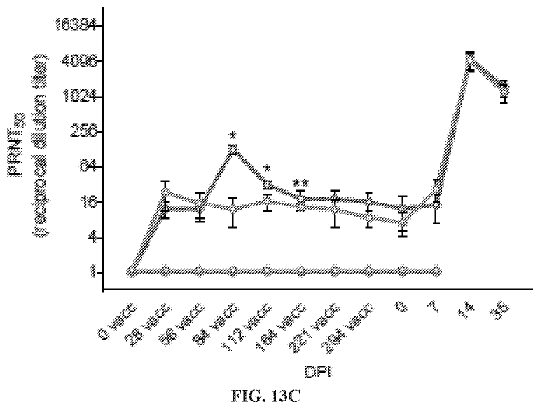

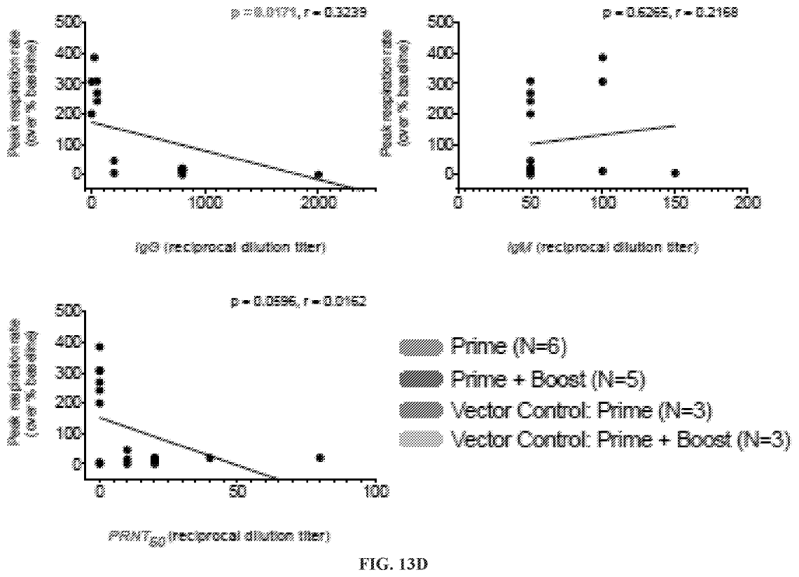

Neutralizing and anti-NiV_{B} G binding antibody titers in vaccinated AGMs. To measure the magnitude and persistence of the humoral response following rVSV-ΔG-NiV_{B} G vaccination, indirect enzyme-linked immunosorbent assays (ELISAs) were performed over the span of a year on serum from immunized AGMs. Neutralizing responses were evaluated by plaque reduction neutralization tests (PRNT₅₀). Anti-NiV-G IgG (1:50-1:1600) (FIG. 13A) and IgM (1:50-1:200) (FIG. 13B) binding titers were generated by 10 days post vaccination. IgM responses waned by 56 days post immunization, while IgG responses slightly decreased but remained stable up until challenge. For IgG responses, the rVSV-ΔG-NiV_{B} G-boosted group only appeared more immunogenic (repeated measures ANOVA Tukey's test; p=0.0345) at 84 days post vaccination (28 days past the booster dose) but by 5-6 months resembled antibody levels in the prime only cohort. All survivors (FIG. 13C) developed neutralizing antibodies that persisted for at least one year. Robust anamnestic binding IgG (1:51,200-1:819,200) and neutralizing titers (1:640-1:10,240) were generated following NiV exposure. In contrast, neither the vector control primed nor boosted groups developed substantial levels of binding or neutralizing antibodies. A negative association was found between respiration rate and IgG levels (p=0.0171, r=0.3239), but not IgM or neutralizing titers, indicating the importance of this isotype in mediating respiratory protection (FIG. 13D).

Cellular responses in vaccinated AGMs. Cellular responses were evaluated over the course of the study by ELISPOT. Minimal NiV G-specific responses were evident in vaccinated AGM peripheral blood mononuclear cells (PBMC), particularly during the vaccination phase. Starting at 7 DPI, low cellular responses were detected in approximately half of prime-only vaccinated subjects (P-1, P-3, P-5), one prime-boosted subject (PB-3), and one of the vector controls (VC-PB-3) (FIG. 14A). By 14 DPI, G-specific IFNγ spot-forming units (SFUs) were visible in wells containing PBMC from all specifically vaccinated AGMs (range of 8 to 212 spot-forming units (SFUs) per million PBMCs) except for subjects P-6 and PB-3.

For a more granular analysis, intracellular cytokine staining (ICS) were performed via flow cytometry to examine the polyfunctionality of CD4+ and CD8+ antigen-specific T cells in each AGM group. The analyses indicated that specifically vaccinated subjects, whether receiving a single or two doses, had higher frequencies of CD3+ CD4+ (FIG. 13B) and CD3+CD8+ T cells following NiV exposure (FIG. 14C). Moreover, prime only and prime-boosted versus vector control subjects exhibited a higher degree of polyfunctionality and expressed more IFNγ for both CD4+ and CD8+ G-specific T cell subsets (FIG. 14D).

As natural killer (NK) cells are implicated in rVSV-mediated protection^{25, 30, 31, 32}, the functional capacity of this subset was also surveyed for each cohort. Specifically immunized subjects expressed higher frequencies of total (CD3− CD8a+), degranulating (CD3− CD8a+ CD107+), and IFNγ-secreting (CD3− CD8a+ IFNγ+) NK cells. Instead, vector controls exhibited an overall decline in these NK cell populations at late disease.

Circulating cytokine detection. As expected, the vector controls expressed higher levels of pro-inflammatory plasma cytokines and growth factors following NiV_{B} exposure, which is in line with ongoing viral replication and clinical disease in these subjects. At 7 DPI, significantly elevated inflammatory mediators in the vector control groups included inflammatory protein-10 (IP-10), monocyte chemoattractant protein-1 (MCP-1), and interferon gamma (IFNγ). Conversely, higher levels of the monocyte chemoattractant, IL-8, were observed in prime and prime+boost groups.

Transcriptional correlates of protection. To dissect molecular signals correlating with rVSV-ΔG-NiV_{B} G-mediated protection, targeted transcriptomics were performed on peripheral whole blood RNA collected from AGMs. Dimensional reduction via principal component analysis (PCA) revealed variation in the dataset was mostly driven by timepoint (0, 4, 7, 10 DPI or the terminal time point in fatal cases) rather than group (prime only, prime+boost, vector control prime, and vector control prime+boost) (FIG. 14A). At 10 DPI or the terminal time point in vector controls, prime and prime+boost groups as well as vector control prime and vector control prime+boost groups clustered similarly indicating similar overall expression patterns in these respective pairings (FIG. 14B). The topmost downregulated (FIG. 14C) and upregulated (FIG. 14D) mRNAs in specifically vaccinated subjects are depicted. Specifically vaccinated versus vector control samples at late disease (10 DPI or the terminal time point in fatal cases) expressed lower levels of transcripts associated with interferon signaling (e.g., MX1, OASL, IFI44, GBP1, IFIT2, IFIT1)(FIG. 13C). Upregulated transcripts were involved in adaptive immunity (e.g., CD96, KLRK1, KLRG1, KLRF1, SH2D1A) (FIG. 13D). Multiple NK-cell associated transcripts were also upregulated in survivors (KRLC3, KLRC2, GZMM), which corroborated the flow cytometry data.

To predict cell-type quantities based on transcriptional signatures, digital cell quantification (DCQ) was performed via nSolver at late disease (FIG. 14E). This analysis predicted survival was associated with increased frequencies of cells involved in adaptive immunity (T-helper 1 (Th1) cells, B cells, CD8 T cells, and cytotoxic cells), corresponding to the differential expression analysis. Conversely, lethality regardless of time of vaccination correlated with increased neutrophil frequencies, which was corroborated by the hematology data (Table 1). Lethality was also associated with an increased abundance of exhausted CD8+ T cells.

As expected, enrichment of upregulated differentially expressed transcripts (BH-adjusted p-value <0.05) in specifically versus non-specifically vaccinated subjects indicated survival correlated with activation of pathways involved in memory responses and immunoregulation, e.g., “immune response-regulating signaling,” “adaptive immune response,” “regulation of lymphocyte activation,” and “regulation of B cell activation” (FIG. 14F).

In summary, rVSV-ΔG-NiV_{B} G is a highly effective and durable vaccine against NiV disease. Several attributes of rVSV-ΔG-NiV_{B} G make it an ideal vaccine candidate. rVSV-based vaccines have been tested in hundreds of NHPs with no signs of toxicity. A vector containing the same backbone (Ervebo) was deemed safe for human use by the US Federal Drug Administration and European Medicines Agency²⁶; however, a minor subset of vaccines developed arthralgia/arthritis, dermatitis, and cutaneous vasculitis in association with viral replication that ultimately resolved.^{41, 42} The rVSV-ΔG-NiV_{B} G vector encodes only one of the two NiV proteins necessary for viral entry and accordingly only undergoes a single round of replication, which may further enhance its safety profile and minimize vaccine-related adverse events such as the case with Ervebo. Pre-existing immunity against the vector backbone is unlikely as VSV seropositivity is low in the general population⁴³, and Marzi et al. showed that previous vaccination with an rVSV vector did not rescind protection following subsequent immunization with another rVSV-based vaccine⁴⁴. Another attractive feature of rVSV-ΔG-NiV_{B} G is its inability as a rhabdovirus to reassort or integrate into the host genome unlike other vectors⁴⁵. Finally, rVSV-ΔG-NiV_{B} G grows rapidly to high titers, facilitating large-scale manufacturing.

As an individual may not encounter a pathogen for years after immunization, vaccines providing long-lasting immunity are needed. One report showed ferrets vaccinated with HeV sG were protected after 14 months⁴⁶. To the inventors' knowledge, this is the first study to investigate the efficacy of a NiV vaccine in primates beyond a month after immunization. The results herein demonstrate that a single injection of rVSV-ΔG-NiV_{B} G provides complete protection in the “gold-standard” NHP model, AGMs¹⁴, for at least one year after vaccination with no adverse reactions. These results are encouraging as a multi-dose vaccine regimen requiring several weeks to generate protective immunity is impractical in an outbreak scenario and creates additional logistical issues. A one-shot vaccine approach is preferable and more economical. Remarkably, no overt clinical illness or detectable viral loads were observed in vaccinated subjects suggesting rVSV-ΔG-NiV_{B} G may induce sterilizing immunity. Vector control AGMs instead exhibited high viral loads and common NiV disease features such as anorexia, hematological and serum biochemistry changes, depression, respiratory distress, and neurological deficits. These animals succumbed within the typical time-to-death for this experimental model (7-9 DPI)¹⁴.

Although the precise mechanisms of rVSV-mediated immunity against NiV disease are not yet understood, it is shown herein that rVSV-ΔG-NiV_{B} G potently activates humoral responses. In this study, G-specific IgG titers in rVSV-ΔG-NiV_{B} G-vaccinated AGMs correlated with respiratory health, indicating the presence of this immune constituent may reliably predict protection. Vaccination induced stable and moderate-to-robust circulating neutralizing and G-specific IgG titers, but only low IgM levels were generated. In both specifically vaccinated groups, binding and neutralizing antibody levels waned half a year after the initial prime dose, but anamnestic IgG (1:12,800-1:819,200) and neutralizing (1:640-1:10,240) titers were generated following NiV_{B} exposure that peaked during convalescence. In contrast, non-specific control antibody levels remained low or below the limit of detection for the assay throughout the entire study. A booster dose transiently augmented antibody responses during the vaccination phase but did not offer additional efficacy and by five months matched levels elicited by a single dose of rVSV-ΔG-NiV_{B} G. Therefore, a booster does not appear essential for at least one year but may provide benefit for longer intervals.

Cellular responses induced by rVSV-ΔG-NiV_{B} G vaccination may play a supportive role in conferring resistance against NiV disease. Flow cytometry results show increased frequencies of total, cytotoxic, and IFNγ-secreting NK cells in PBMC samples from specifically immunized subjects in the present study, in addition to the expression of NK cell-associated transcripts. In humans, the Ervebo vaccine was reported to modulate CD56+ NK cell counts and the expression of various NK surface receptors such as NKG2D, NKp30, and killer immunoglobulin-like receptors shortly after vaccination. A systems vaccinology approach also demonstrated the total frequency of CD56+ NK cell count and CXCR6 expression on NK cells correlated with the antibody response to Ervebo in healthy adults⁴⁸. Thus, NK cells may contribute to rVSV protection in myriad ways. Other cellular effectors such as helper and effector T cells may also participate in host defense. Specific IFNγ immunospot assay responses directed at the NiV_{B} G, albeit modest, were observed in 5/6 prime only and 4/5 prime-boosted subjects over the course of the study. Digital cell quantitation via whole blood transcriptomics corroborated a predicted increase in circulating Th1 and CD8 T cell frequencies in specifically immunized AGMs. Moreover, the inventors detected a higher abundance of total CD4+ T and CD8+ T-cell counts, and higher antigen-specific T cell polyfunctionality, in rVSV-ΔG-NiV_{B} G-vaccinated subjects.

Thus, the rVSV-ΔG-NiV_{B} G vaccine provides durable protection against NiV disease by inducing long-lived adaptive responses. This vaccine will be a useful tool in curtailing future outbreaks of the virus as still near annual cases are reported in India and Bangladesh with high mortality rates. Breakthrough infections have been commonly experienced following COVID-19 vaccination partly because of waning immunity⁴⁹, which may fuel vaccine hesitancy. COVID-19 vaccines so far have also failed to provide sterilizing protection. It is demonstrated herein that rVSV-ΔG-NiV_{B} G provides durable immunity with no detectable NiV replication, which may bolster public confidence in the vaccine. Future work will include manufacturing clinical grade vaccine lots, determining optimal dosing regimens, and improving temperature stability as currently rVSV-ΔG-NiV_{B} G requires −80° C. long-term storage. Evaluating efficacy at longer gaps between vaccination and challenge should also be conducted to inform public health policy decision-making, e.g., timing of booster vaccinations and effectively responding to a pandemic.

Materials and Methods. Characterization of rVSV-ΔG-NiV_{B}G vaccine. The rVSV-ΔG-NiV_{B} G vaccine was recovered, sequenced, and characterized as described previously²⁵. The vaccine stocks tested negative for Mycoplasma and endotoxin contamination.

Challenge virus. The NiV_{B} challenge material used in the study (200401066) originated from a fatal human case during an outbreak in Rajbari, Bangladesh in 2004. The challenge material was passaged twice onto Vero E6 cells and supernatants were collected and stored at −80° C. as ˜1 ml aliquots. Four distinct mutations of sufficient frequency were found between the P2 stock of NiV_{B} and the reference genome (GenBank Accession number AY988601.1). One mutation was non-coding whereas the remaining mutations encode for three single amino acid changes: one in the M protein and two in the F protein⁶. No detectable Mycoplasma or endotoxin were present in the virus seed stock (<0.5 EU/ml).

Ethics statement. Monkeys were handled in animal BSL-2 and BSL-4 containment in the Galveston National Laboratory (GNL) at the University of Texas Medical Branch (UTMB), Galveston, Texas. This facility is Office of Laboratory Welfare (OLAW) assured and fully accredited by the Association for the Assessment and Accreditation of Laboratory Animal Care International (AALAC). All research was approved by the UTMB Institutional Animal Care and Use Committee (IACUC) and complied with the Animal Welfare Act and other federal statutes and regulations pertaining to animal experimentation. Provisions were taken to prevent, ameliorate, and minimize pain and distress of the animals. Animals were monitored by an attending veterinarian and scored at least twice daily for food intake, responsiveness, weakness, recumbency, labored breathing, diarrhea, edema, dehydration, and the presence of coagulopathies. Animals meeting humane endpoint scoring criteria were promptly euthanized with a pentobarbital solution.

NHP vaccination and challenge. Seventeen healthy, adult AGMs (8 males and 9 females) from St. Kitts (Chlorocebus aethiops; Worldwide Primates) were randomized into four groups: prime only (N=6), prime+boost (N=5), vector control prime (N=3), and vector control prime+boost (N=3). The six experimental animals were specifically vaccinated by intramuscular (i.m.) injection of 1×10⁷ PFU of rVSV-ΔG-NiV_{B} G, and control animals were vaccinated by i.m. injection of 1×10⁷ PFU of the nonspecific vector. One subject in the prime+boost group was euthanized due to issues deemed unrelated to the study or vaccination. One year after prime vaccination, the remaining seventeen AGMs were exposed to 5×10³ PFU of NiV_{B} i.n. using the LMA Mucosal Atomization Device (MAD) as previously described²⁹.

All animals for both studies were given physical examinations, and blood was collected before vaccination (day 0); and on days 4, 7, 10, 14/15, 21, 28, and 35 after virus challenge. The AGMs were monitored daily and scored for disease progression with an internal NiV humane endpoint scoring sheet approved by the UTMB IACUC. Scoring criteria was based on parameters such as respiration (0-9), appetite (0-2), activity/appearance (0-9), and neurological signs (0-9). A score > or equal to 9 met euthanasia criteria. UTMB facilities used in this work are accredited by the Association for Assessment and Accreditation of Laboratory Animal Care International and adhere to principles specified in the eighth edition of the Guide for the Care and Use of Laboratory Animals, National Research Council. The scoring changes measured from baseline included posture and activity level, attitude and behavior, food intake, respiration, and central nervous system abnormalities.

Hematology and serum biochemistry. Total red blood cell counts, white blood cell counts, white blood cell differentials, platelet counts, hematocrit values, total hemoglobin concentrations, mean cell volumes, mean corpuscular volumes, and mean corpuscular hemoglobin concentrations were analyzed from blood collected in tubes containing EDTA using a Vetscan HM5 laser based hematologic analyzer (Zoetis). Serum samples were tested for concentrations of albumin, amylase, alanine aminotransferase (ALT), aspartate aminotransferase (AST), alkaline phosphatase (ALP), gamma-glutamyltransferase (GGT), blood urea nitrogen (BUN), creatinine (CRE), C-reactive protein (CRP), calcium, glucose, total protein, and uric acid by using a Piccolo point-of-care analyzer and Biochemistry Panel Plus analyzer discs (Abaxis).

RNA isolation from NiV_{B}-infected AGMs. On the specified procedure days, 100 μl of blood was added to 600 μl of AVL viral lysis buffer (Qiagen) for RNA extraction. For tissues, ˜100 mg of sample was stored in 1 ml RNAlater (Qiagen) for 7 days for stabilization. RNAlater was removed and tissues were homogenized in 600 μl RLT buffer (Qiagen) in a 2 mL cryovial using a Tissue Lyser (Qiagen) and ceramic beads. The tissues sampled included axillary, inguinal, mandibular, and mesenteric lymph nodes; upper, middle, and lower lobes of both left and right lungs; spleen; liver; kidney; adrenal gland; frontal cortex of brain; brainstem; cervical spinal cord; submandibular salivary gland; tonsil; heart; duodenum; pancreas; ileocecal junction; transverse colon; urinary bladder; ovary or testis; uterus or prostate; nasal mucosa; conjunctiva; and eye. All blood samples were inactivated in AVL viral lysis buffer, and tissue samples were homogenized and inactivated in RLT buffer prior to removal from the BSL-4 laboratory. Subsequently, RNA was isolated from blood using the QIAamp viral RNA kit (Qiagen), and from tissues using the RNeasy minikit (Qiagen), according to the vendor instructions supplied with each kit.

Quantification of viral load. Viral loads of RNA from blood or tissues were measured using reverse transcriptase quantitative PCR (RT-qPCR) and primers/probe targeting the N gene and intergenic region between N and P of NiV_{B}. Probe sequences were 6FAM-5′CGTCACACATCAGCTCTGACAA 3′-6TAMRA for NiV_{B} (Life Technologies, Carlsbad, CA). Threshold cycle (CT) values representing viral genomes were analyzed with CFX Manager software; the data are displayed as genome equivalents (GEq). To create the GEq standard, RNA from viral stocks was extracted, and the number of genomes present was calculated using Avogadro's number and the molecular weight of the genome.

Virus titration was performed by plaque assay using Vero 76 cells (ATCC CRL-1587) from all plasma samples. Briefly, increasing 10-fold dilutions of the samples were adsorbed to Vero 76 cell monolayers in duplicate wells (200 μl/well) and overlaid with 0.8% agarose in 1× Minimum Essentials Medium (MEM) with 5% FBS and 1% penicillin/streptomycin. After a 2-3-day incubation at 37° C./5% CO₂, neutral red stain was added, and plaques were counted following an additional 24-hour incubation. The limit of detection for this assay is 25 PFU/mL.

ELISA. Sera collected at the indicated time points were tested for total anti-NiV IgG and IgM antibodies by ELISA using monkey species-specific kits (Alpha Diagnostic International #NIV-015 and #NIV-020) following the vendor recommendations.

Plaque reduction neutralization test. Neutralization titers were calculated by determining the dilution of serum that reduced 50% of plaques (PRNT₅₀). A standard 100 PFU amount of NiV_{B} was incubated with two-fold serial dilutions of serum samples in Dulbecco's Modified Eagle Medium (DMEM) for one hour. The virus-serum mixture was then used to inoculate Vero 76 cells for 30 minutes. Cells were overlaid with 2×MEM agar medium, incubated for 2-3 days, and plaques counted after 24 hours of 5% neutral red staining.

RNA sample preparation for transcriptomic analyses. NHPV2_Immunology reporter and capture probesets (Nanostring Technologies) were hybridized with ˜3 μl of blood RNA at 65° C. for ˜24 hours as previously described⁵⁰. Following the hybridization, the RNA: probe set complexes were loaded into an nCounter® microfluidics cartridge and assayed on a NanoString nCounter® SPRINT Profiler. To estimate the abundance of each of the 769 unique mRNA immune-related targets included in the NHPV2_Immunology panel, fluorescent reporter barcodes were imaged and counted for each sample lane.

Bioinformatic analysis. The nCounter® RCC files were imported into NanoString nSolver® 4.0 software. All samples met the integrated quality control criteria. To compensate for varying RNA inputs, housekeeping genes and spiked-in positive and negative controls were incorporated to normalize raw counts. The data were analyzed using the NanoString nSolver® Advanced Analysis 2.0 package to generate principal-component analysis figures and differential expression heatmaps. A full list of probes along with log fold changes and p-values are supplied as Data S1. Normalized data (log fold change values and BH-adjusted p-values) for each sample group was exported as a .CSV file (Microsoft Excel Office for Mac v.14.1.0). MetaScape⁵¹ was used for pathway analysis of differentially expressed transcripts (BH-adjusted p-value <0.05 for the prime versus vector control group) using human annotations and the default settings (3 minimum overlap, 1.5 minimum enrichment). GraphPad Prism v.9 was used to produce heat maps. Human annotations were added for each respective gene to perform immune cell profiling and generate cell-type plots within nSolver®.

Histology. Tissue sections were deparaffinized and rehydrated through xylene and graded ethanol washes. Slides went through heat antigen retrieval in a steamer at 95° C. for 20 minutes in Sigma Citrate Buffer, pH6.0, 10× (Sigma Aldrich, St. Louis, MO). To block endogenous peroxidase activity, slides were treated with a 3% hydrogen peroxide and rinsed in distilled water. The tissue sections were processed for IHC using the Thermo Autostainer 360 (ThermoFisher, Kalamazoo, MI). Sequential 15-minute incubations with avidin D and biotin solutions (Vector Laboratories, Burlingame, CA #SP-2001) were performed to block endogenous biotin reactivity. Specific anti-NiV immunoreactivity was detected using an Anti-NiV primary antibody at a 1:4000 dilution for 60 min. Secondary antibody used was biotinylated goat anti-rabbit IgG (Vector Laboratories, Burlingame, CA #BA-1000) at 1:200 for 30 min followed by Vector Horseradish Peroxidase Streptavidin, R.T.U (Vector Laboratories #SA-5704) for 30 min. Slides were developed with Dako DAB chromogen (Dako, Carpenteria, CA #K3468) for 5 min and counterstained with hematoxylin for 45 seconds.

ELISpot. To analyze cellular responses, NHP PBMCs were rapidly thawed in a water bath at 37° C. and resuspended in pre-warmed complete RPMI 1640 medium with 10% FBS, 1% GlutaMAX (Thermo Fisher Scientific) and 1% penicillin-streptomycin (Thermo Fisher Scientific). Cells were counted and rested overnight at 37° C. and 5% CO2. After the resting period, PBMCs were either left unstimulated or stimulated for ˜24 h at 37° C. and 5% CO2 with either lectin (Sigma-Aldrich) from Phytolacca americana (PWM) or a custom NiV_{B} G peptide pool (GenScript) spanning the length of G. The NiV_{B} G peptide pool contained 148×15mer peptides with 11 amino acid overlaps. The lyophilized pool was prepared in dimethyl sulfoxide and used at a final concentration of 2 μg/mL, whereas unstimulated cells contained 0.2% dimethyl sulfoxide by volume. As a positive stimulation control, PBMCs were stimulated with PWM at a final concentration of 0.5 μg/mL. For ELISpot analysis, samples were stained using a single-color primate IFN-γ kits (mAB Biotech) according to the manufacturer's recommendations. PBMCs were plated in duplicate at 2.5×10⁵ cells per well in a 96-well plate coated with NHP IFN-γ capture antibody. Following a ˜24-hour incubation at 37° C. and 5% CO2, ELISpot plates were air dried and imaged using an Immunospot S6 UNIVERSAL Analyzer (Cellular Technology Limited). Reported values were calculated by subtracting the number of SFCs in each unstimulated sample from its respective stimulated counterpart at the corresponding DPI.

Bead-based multiplex assays. Plasma analytes were measured by flow cytometry using Biolegend LegendPlex™ assays and a FACS Canto II cytometer (Becton Dickson). Samples were processed in duplicate using a BioLegend Nonhuman Primate Inflammation 13-plex kit (1:4 dilution) according to the manufacturer's instructions. To ensure consistency among plates, standards were mixed in batch and aliquoted across all plates. Optional wash steps were incorporated to reduce background. Fold change calculations were plotted using the package Pheatmap v1.0.12 in R. Results of fold change calculations and ANOVA with Tukey post-hoc tests were calculated and plotted using ggplot2 (v3.3.5)⁵², ggbreak (v0.0.8)⁵³, viridis (v0.6.2)⁵⁴, and rstatix (v0.7.0) packages.

Statistics. The survival of prime only (specific versus control) and prime-boosted (specific versus control) groups was compared using a log-rank test. Statistical tests were performed using Prism 9 (GraphPad). All data is derived from a single animal experiment. Statistics were derived from average values from the following four cohorts: prime only (N=6), prime+boost (N=5), vector control prime (N=3), and vector control prime+boost (N=3). Statistics for all figures were calculated from individual animal data values rather than technical replicates. For experiments with technical replicates (for example, duplicate RT-qPCR reactions/wells), only the mean was used to calculate statistical significance. A two-way analysis of variance (ANOVA) with Tukey's multi-comparisons test was used to determine statistical significance between prime only (specific versus control) and prime-boosted (specific versus control) groups for viral loads, humoral responses, and cellular responses. For cytokine bead array measurements, the results of fold change calculations and ANOVA with Tukey post-hoc test were calculated using the rstatix (v0.7.0) package. A multiple hypothesis Benjamini-Hochberg-corrected p-value <0.05 was deemed significant for transcriptomic analyses.

Representative photomicrographs were qualitatively considered to display lesions that were nominally or ordinally measured by masking of the pathologist after examination and ranking lesions to satisfy study objectives. Additionally, a thorough examination of multiple slides of target tissues (for example 18 slides of lung) multiple times (up to three times per tissue) was performed in a timely manner to maintain interpretation consistency.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES—EXAMPLE 1

1. Arunkumar, G. et al. Outbreak Investigation of Nipah Virus Disease in Kerala, India, 2018. Journal of Infectious Diseases 219, 1867-1878 (2019).
2. News Desk. Nipah virus: Bangladesh IEDCR reports 6 cases, 4 deaths. Outbreak News Today (2020).
3. Gurley, E. S. et al. Person-to-Person Transmission of Nipah Virus in a Bangladeshi Community. Emerging Infectious Diseases 13, 1031-1037 (2007).
4. Luby, S. P., Gurley, E. S. & Hossain, M. J. Transmission of human infection with nipah virus. Clinical Infectious Diseases vol. 49 1743-1748 (2009).
5. Amaya, M. & Broder, C. C. Annual Review of Virology Vaccines to Emerging Viruses: Nipah and Hendra. Annu. Rev. Virol 7, 447-473 (2020).
6. Zhu, Z. et al. Exceptionally potent cross-reactive neutralization of Nipah and Hendra viruses by a human monoclonal antibody. Journal of Infectious Diseases 197, 846-853 (2008).
7. Zhu, Z. et al. Potent Neutralization of Hendra and Nipah Viruses by Human Monoclonal Antibodies. Journal of Virology 80, 891-899 (2006).
8. Chattopadhyay, A. & Rose, J. K. Complementing Defective Viruses That Express Separate Paramyxovirus Glycoproteins Provide a New Vaccine Vector Approach. Journal of Virology 85, 2004-2011 (2011).
9. Lo, M. K. et al. Single-dose replication-defective VSV-based Nipah virus vaccines provide protection from lethal challenge in Syrian hamsters. Antiviral Research 101, 26-29 (2014).
10. Mire, C. E. et al. Single injection recombinant vesicular stomatitis virus vaccines protect ferrets against lethal Nipah virus disease. Virology Journal 10, 353-undefined (2013).
11. Mire, C. E. et al. Use of single-injection recombinant vesicular stomatitis virus vaccine to protect nonhuman primates against lethal nipah virus disease. Emerging Infectious Diseases 25, 1144-1152 (2019).
12. DeBuysscher, B. L., Scott, D., Marzi, A., Prescott, J. & Feldmann, H. Single-dose live-attenuated Nipah virus vaccines confer complete protection by eliciting antibodies directed against surface glycoproteins. Vaccine 32, 2637-2644 (2014).
13. Prescott, J. et al. Single-dose live-attenuated vesicular stomatitis virus-based vaccine protects African green monkeys from Nipah virus disease. Vaccine 33, 2823-2829 (2015).
14. Debuysscher, B. L., Scott, D., Thomas, T., Feldmann, H. & Prescott, J. Peri-exposure protection against Nipah virus disease using a single-dose recombinant vesicular stomatitis virus-based vaccine. npj Vaccines 1, (2016).
15. Kurup, D., Wirblich, C., Feldmann, H., Marzi, A. & Schnell, M. J. Rhabdovirus-Based Vaccine Platforms against Henipaviruses. Journal of Virology 89, 144-154 (2015).
16. Publicover, J., Ramsburg, E. & Rose, J. K. A Single-Cycle Vaccine Vector Based on Vesicular Stomatitis Virus Can Induce Immune Responses Comparable to Those Generated by a Replication-Competent Vector. Journal of Virology 79, 13231-13238 (2005).
17. Geisbert, T. W. et al. Therapeutic Treatment of Nipah Virus Infection in Nonhuman Primates with a Neutralizing Human Monoclonal Antibody. Science Translational Medicine 6, (2014).
18. Mire, C. E. et al. Pathogenic Differences between Nipah Virus Bangladesh and Malaysia Strains in Primates: Implications for Antibody Therapy. Scientific Reports 6, (2016).
19. Whitt, M. A. Generation of VSV pseudotypes using recombinant ΔG-VSV for studies on virus entry, identification of entry inhibitors, and immune responses to vaccines. Journal of Virological Methods 169, 365-374 (2010).
20. Gibson, D. G. et al. Enzymatic assembly of DNA molecules up to several hundred kilobases. Nature Methods 6, 343-345 (2009).
21. Mire, C. E. et al. Recombinant vesicular stomatitis virus vaccine vectors expressing filovirus glycoproteins lack neurovirulence in nonhuman primates. PLoS Neglected Tropical Diseases 6, (2012).
22. Pallister, J. A. et al. Vaccination of ferrets with a recombinant G glycoprotein subunit vaccine provides protection against Nipah virus disease for over 12 months. Virology Journal 10, (2013).
23. McEachern, J. A. et al. A recombinant subunit vaccine formulation protects against lethal Nipah virus challenge in cats. Vaccine 26, 3842-3852 (2008).
24. Bossart, K. N. et al. A Hendra Virus G Glycoprotein Subunit Vaccine Protects African Green Monkeys from Nipah Virus Challenge. Science Translational Medicine 4, (2012).
25. Geisbert, T. W. et al. A single dose investigational subunit vaccine for human use against Nipah virus and Hendra virus. npj Vaccines 6, (2021).

### REFERENCES—EXAMPLE 2

1. WHO. Fact-sheets: Nipah virus. 2018 [cited] Available from: https://www.who.int/news-room/fact-sheets/detail/nipah-virus
2. Luby, S. P. The pandemic potential of Nipah virus. Antiviral Research 100, 38-43 (2013).
3. Gómez Roman, R. et al. Medical countermeasures against henipaviruses: a review and public health perspective. The Lancet Infectious Diseases 22, e13-e27 (2022).
4. CDC/USDA. Federal Select Agent Program: HHS and USDA Select Agents and Toxins 7CFR Part 331, 9 CFR Part 121, and 42 CFR Part 73. 2021 [cited] Available from: https://www.selectagents.gov/sat/list.htm?CDC_AA_refVal=https%3A%2F%2Fwww.selectagents.gov%2FSelectAgentsandToxinsList.html
5. Sun, B., Jia, L., Liang, B., Chen, Q. & Liu, D. Phylogeography, Transmission, and Viral Proteins of Nipah Virus. Virol Sin 33, 385-393 (2018).
6. Mire, C. E. et al. Pathogenic Differences between Nipah Virus Bangladesh and Malaysia Strains in Primates: Implications for Antibody Therapy. Scientific Reports 6, 30916 (2016).
7. Wang, Z. et al. Architecture and antigenicity of the Nipah virus attachment glycoprotein. Science 375, 1373-1378 (2022).
8. Guillaume, V. et al. Nipah Virus: Vaccination and Passive Protection Studies in a Hamster Model. Journal of Virology 78, 834-840 (2004).
9. Weingartl, H. M. et al. Recombinant nipah virus vaccines protect pigs against challenge. Journal of virology 80, 7929-7938 (2006).
10. Van Doremalen, N. et al. A single-dose ChAdOx1-vectored vaccine provides complete protection against Nipah Bangladesh and Malaysia in Syrian golden hamsters. PLOS Neglected Tropical Diseases 13, e0007462 (2019).
11. Ploquin, A. et al. Protection against henipavirus infection by use of recombinant adeno-associated virus-vector vaccines. The Journal of infectious diseases 207, 469-478 (2013).
12. Walpita, P. et al. A VLP-based vaccine provides complete protection against Nipah virus challenge following multiple-dose or single-dose vaccination schedules in a hamster model. npj Vaccines 2 (2017).
13. Lo, M. K. et al. Evaluation of a Single-Dose Nucleoside-Modified Messenger RNA Vaccine Encoding Hendra Virus-Soluble Glycoprotein Against Lethal Nipah virus Challenge in Syrian Hamsters. The Journal of Infectious Diseases 221, S493-S498 (2020).
14. Geisbert, T. W. et al. Development of an Acute and Highly Pathogenic Nonhuman Primate Model of Nipah Virus Infection. PLoS ONE 5, e10690 (2010).
15. Yoneda, M. et al. Recombinant Measles Virus Vaccine Expressing the Nipah Virus Glycoprotein Protects against Lethal Nipah Virus Challenge. PLoS ONE 8, e58414 (2013).
16. Bossart, K. N. et al. A Hendra Virus G Glycoprotein Subunit Vaccine Protects African Green Monkeys from Nipah Virus Challenge. Science Translational Medicine 4, 146ra107-146ra141 (2012).
17. Geisbert, T. W. et al. A single dose investigational subunit vaccine for human use against Nipah virus and Hendra virus. npj Vaccines 6 (2021).
18. Eldridge, J. H. et al. A Brighton Collaboration standardized template with key considerations for a benefit/risk assessment for a soluble glycoprotein vaccine to prevent disease caused by Nipah or Hendra viruses. Vaccine 39, 5436-5441 (2021).
19. Debuysscher, B. L., Scott, D., Marzi, A., Prescott, J. & Feldmann, H. Single-dose live-attenuated Nipah virus vaccines confer complete protection by eliciting antibodies directed against surface glycoproteins. Vaccine 32, 2637-2644 (2014).
20. DeBuysscher, B. L., Scott, D., Thomas, T., Feldmann, H. & Prescott, J. Peri-exposure protection against Nipah virus disease using a single-dose recombinant vesicular stomatitis virus-based vaccine. NPJ vaccines 1, 16002 (2016).
21. Prescott, J. et al. Single-dose live-attenuated vesicular stomatitis virus-based vaccine protects African green monkeys from Nipah virus disease. Vaccine 33, 2823-2829 (2015).
22. Chattopadhyay, A. & Rose, J. K. Complementing defective viruses that express separate paramyxovirus glycoproteins provide a new vaccine vector approach. Journal of virology 85, 2004-2011 (2011).
23. Lo, M. K. et al. Single-dose replication-defective VSV-based Nipah virus vaccines provide protection from lethal challenge in Syrian hamsters. Antiviral research 101, 26-29 (2014).
24. Mire, C. E. et al. Single injection recombinant vesicular stomatitis virus vaccines protect ferrets against lethal Nipah virus disease. Virol J; 2013. p. 353.
25. Foster, S. L. et al. A recombinant VSV-vectored vaccine rapidly protects nonhuman primates against lethal Nipah virus disease. Proceedings of the National Academy of Sciences 119 (2022).
26. Woolsey, C. & Geisbert, T. W. Current state of Ebola virus vaccines: A snapshot. PLOS Pathogens 17, e1010078 (2021).
27. Henao-Restrepo, A. M. et al. Efficacy and effectiveness of an rVSV-vectored vaccine in preventing Ebola virus disease: final results from the Guinea ring vaccination, open-label, cluster-randomised trial (Ebola Ça Suffit!). The Lancet 389, 505-518 (2017).
28. WHO. Preliminary results on the efficacy of rVSV-ZEBOV-GP Ebola vaccine using the ring vaccination strategy in the control of an Ebola outbreak in the Democratic Republic of the Congo: an example of integration of research into epidemic response. 10 Apr. 2019 meeting report. 2022 [cited] Available from: https://www.who.int/csr/resources/publications/ebola/ebola-ring-vaccination-results-12-april-2019.pdf
29. Geisbert, J. B. et al. An Intranasal Exposure Model of Lethal Nipah Virus Infection in African Green Monkeys. J Infect Dis 221, S414-S418 (2020).
30. Cross, R. W. et al. A recombinant VSV-vectored vaccine rapidly protects nonhuman primates against heterologous lethal Lassa fever. Cell Rep 40, 111094 (2022).
31. Woolsey, C. et al. A highly attenuated Vesiculovax vaccine rapidly protects nonhuman primates against lethal Marburg virus challenge. PLOS Neglected Tropical Diseases 16, e0010433 (2022).
32. Woolsey, C. et al. Immune correlates of post exposure vaccine protection against Marburg virus. Scientific Reports 10 (2020).
33. Daddario-DiCaprio, K. M. et al. Cross-protection against Marburg virus strains by using a live, attenuated recombinant vaccine. J Virol 80, 9659-9666 (2006).
34. Geisbert, T. W. et al. Vesicular stomatitis virus-based vaccines protect nonhuman primates against aerosol challenge with Ebola and Marburg viruses. Vaccine 26, 6894-6900 (2008).
35. Geisbert, T. W. et al. Vesicular stomatitis virus-based ebola vaccine is well-tolerated and protects immunocompromised nonhuman primates. PLoS Pathog 4, e1000225 (2008).
36. Geisbert, T. W. et al. Single-injection vaccine protects nonhuman primates against infection with marburg virus and three species of ebola virus. J Virol 83, 7296-7304 (2009).
37. Jones, S. M. et al. Live attenuated recombinant vaccine protects nonhuman primates against Ebola and Marburg viruses. Nat Med 11, 786-790 (2005).
38. Qiu, X. et al. Mucosal immunization of cynomolgus macaques with the VSVDeltaG/ZEBOVGP vaccine stimulates strong ebola GP-specific immune responses. PLoS One 4, e5547 (2009).
39. Mire, C. E. et al. Recombinant vesicular stomatitis virus vaccine vectors expressing filovirus glycoproteins lack neurovirulence in nonhuman primates. PLoS Negl Trop Dis 6, e1567 (2012).
40. Marzi, A. et al. Antibodies are necessary for rVSV/ZEBOV-GP-mediated protection against lethal Ebola virus challenge in nonhuman primates. Proc Natl Acad Sci USA 110, 1893-1898 (2013).
41. Huttner, A. et al. The effect of dose on the safety and immunogenicity of the VSV Ebola candidate vaccine: a randomised double-blind, placebo-controlled phase 1/2 trial. The Lancet Infectious Diseases 15, 1156-1166 (2015).
42. Agnandji, S. T. et al. Phase 1 Trials of rVSV Ebola Vaccine in Africa and Europe. New England Journal of Medicine 374, 1647-1660 (2016).
43. Roberts, A., Buonocore, L., Price, R., Forman, J. & Rose, J. K. Attenuated Vesicular Stomatitis Viruses as Vaccine Vectors. Journal of Virology 73, 3723-3732 (1999).
44. Marzi, A., Feldmann, F., Geisbert, T., Feldmann, H. & Safronetz, D. Vesicular Stomatitis Virus-Based Vaccines against Lassa and Ebola Viruses. Emerging Infectious Disease journal 21, 305 (2015).
45. Geisbert, T. W. & Feldmann, H. Recombinant vesicular stomatitis virus-based vaccines against Ebola and Marburg virus infections. J Infect Dis 204 Suppl 3, S1075-1081 (2011).
46. Pallister, J. A. et al. Vaccination of ferrets with a recombinant G glycoprotein subunit vaccine provides protection against Nipah virus disease for over 12 months. Virol J 10, 237 (2013).
47. Marzi, A. et al. VSV-EBOV rapidly protects macaques against infection with the 2014/15 Ebola virus outbreak strain. Science 349, 739-742 (2015).
48. Pejoski, D. et al. Rapid dose-dependent Natural Killer (NK) cell modulation and cytokine responses following human rVSV-ZEBOV Ebolavirus vaccination. npj Vaccines 5 (2020).
49. Goldberg, Y. et al. Protection and Waning of Natural and Hybrid Immunity to SARS-CoV-2. New England Journal of Medicine 386, 2201-2212 (2022).
50. Woolsey, C. et al. Bundibugyo ebolavirus Survival Is Associated with Early Activation of Adaptive Immunity and Reduced Myeloid-Derived Suppressor Cell Signaling. mBio 12, e0151721 (2021).
51. Zhou, Y. et al. Metascape provides a biologist-oriented resource for the analysis of systems-level datasets. Nat Commun 10, 1523 (2019).
52. Wickham, H. Ggplot2: Elegant Graphics for Data Analysis. Springer-Verlag: New York, 2018.
53. Xu, S. et al. Use ggbreak to Effectively Utilize Plotting Space to Deal With Large Datasets and Outliers. Frontiers in Genetics 12 (2021).
54. Garnier, S., Ross, Noam, Rudis, Robert, Camargo, Pedro A, Sciaini, Marco, Scherer, Cédric viridis—Colorblind-Friendly Color Maps for R. Zenodo R. doi: 10.5281/zenodo.4679424, R package version 0.6.2, https://sjmgarnier.github.io/viridis/. (2021).

What is claimed is:

1. A composition or vaccine comprising a recombinant vesicular stomatitis virus (rVSV) viral vector that expresses a Nipah Virus protein, wherein the rVSV vector comprises one or more heterologous polynucleotides coding for and expressing a Nipah Virus NiV_{B} G-protein; wherein the NiV_{B} G-protein antigen comprises an amino acid sequence as set forth in SEQ ID NO: 6, or wherein the heterologous polynucleotide encodes a polypeptide coding for the NiV_{B} G-protein antigen comprising at least 90% sequence identity to an amino acid sequence as set forth in SEQ ID NO: 6, or 90% sequence identity to a nucleic acid sequence as set forth in SEQ ID NO: 2, wherein the composition or vaccine is effective to reduce or prevent a Nipah Virus infection at 3 days against both Malaysia strain (NiV_{M}) and Bangladesh strain (NiV_{B}).

2. The composition or vaccine of claim 1, wherein the polynucleotide has a 91, 92, 93, 94, 95, 96, 97, 98, 99, or 100% sequence identity to the nucleic acid sequence as set forth in SEQ ID NO: 2.

3. The composition or vaccine of claim 1, wherein the polynucleotide has a 91, 92, 93, 94, 95, 96, 97, 98, 99, or 100% sequence identity to the sequence as set forth in SEQ ID NO: 6.

4. The composition or vaccine of claim 1, wherein the composition or vaccine is effective to reduce or prevent a Nipah Virus infection with a single dose at 3 days.

5. The composition or vaccine of claim 1, wherein the polynucleotide encoding the NiV_{B} G-protein antigen is operably linked to a promoter selected from the group consisting of an immediate early cytomegalovirus (CMV) promoter, guinea pig CMV promoter, an SV40 promoter, Human Herpesvirus Type III glycoprotein B (HHV3gB) promoter, Pseudorabies Virus promoters, glycoprotein X promoter, Herpes Simplex Virus-1 alpha 4 promoter, a Marek's Disease Virus glycoprotein A (or gC) promoter, a Marek's Disease Virus glycoprotein B promoter, a Marek's Disease Virus glycoprotein E promoter, a Marek's Disease Virus glycoprotein I promoter, an Infectious Laryngotracheitis Virus glycoprotein B, an Infectious Laryngotracheitis Virus glycoprotein E promoter, an Infectious Laryngotracheitis Virus glycoprotein D promoter, an Infectious Laryngotracheitis Virus glycoprotein 1 promoter, vaccinia H6, and a combination thereof.

6. The composition or vaccine of claim 1, wherein the polynucleotide encoding the NiV_{B} G-protein is inserted between a VSV-M protein and a VSV-L protein on a VSV genome.

7. The composition or vaccine of claim 1, wherein the composition or vaccine further comprises a pharmaceutically or veterinarily acceptable carrier, excipient, vehicle or adjuvant.

8. The composition or vaccine of claim 1, wherein the composition or vaccine does not comprise a Green Fluorescent Protein protein or gene.

9. The composition or vaccine of claim 1, wherein an immune response occurs within 3 days and provides protection at 7 days for Malaysian Nipah, Bangladesh Nipah, or both.

10. A method of vaccinating an animal or for inducing an immunogenic or protective response in an animal against avian influenza pathogens, comprising at least one administration of the composition of claim 1.

11. The method of claim 10, wherein the polynucleotide has a 91, 92, 93, 94, 95, 96, 97, 98, 99, or 100% sequence identity to the nucleic acid sequence as set forth in SEQ ID NO: 2.

12. The method of claim 10, wherein the polynucleotide has a 91, 92, 93, 94, 95, 96, 97, 98, 99, or 100% sequence identity to the amino acid sequence as set forth in SEQ ID NO: 6.

13. The method of claim 10, wherein the Nipah Virus is a Malaysia strain (NiV_{M}), or a Bangladesh strain (NiV_{B}).

14. The method of claim 10, wherein the composition or vaccine is effective to reduce or prevent a Nipah Virus infection with a single dose at 3 days.

15. The method of claim 10, wherein the polynucleotide encoding the NiV_{B} G-protein antigen is operably linked to a promoter selected from the group consisting of an immediate early cytomegalovirus (CMV) promoter, guinea pig CMV promoter, an SV40 promoter, Human Herpesvirus Type III glycoprotein B (HHV3gB) promoter, Pseudorabies Virus promoters, glycoprotein X promoter, Herpes Simplex Virus-1 alpha 4 promoter, a Marek's Disease Virus glycoprotein A (or gC) promoter, a Marek's Disease Virus glycoprotein B promoter, a Marek's Disease Virus glycoprotein E promoter, a Marek's Disease Virus glycoprotein I promoter, an Infectious Laryngotracheitis Virus glycoprotein B, an Infectious Laryngotracheitis Virus glycoprotein E promoter, an Infectious Laryngotracheitis Virus glycoprotein D promoter, an Infectious Laryngotracheitis Virus glycoprotein I promoter, vaccinia H6, and a combination thereof.

16. The method of claim 10, wherein the polynucleotide encoding the NiV_{B} G-protein is inserted between a VSV-M protein and a VSV-L protein on a VSV genome.

17. The method of claim 10, wherein the composition or vaccine further comprises a pharmaceutically or veterinarily acceptable carrier, excipient, vehicle or adjuvant.

18. The method of claim 10, wherein an immune response occurs within 3 days and provides protection at 7 days for Malaysian Nipah, Bangladesh Nipah, or both.

19. The method of claim 10, wherein the administration further comprises a prime-boost administration regimen.

20. The method of claim 10, wherein the animal is a human.

21. The method of claim 10, wherein the composition or vaccine does not comprise a Green Fluorescent Protein gene.

22. A recombinant viral vector composition or vaccine comprising a recombinant vesicular stomatitis virus (rVSV) viral vector that expresses a Nipah Virus protein, wherein the rVSV vector comprises one or more heterologous polynucleotides coding for and expressing a Nipah Virus NiV_{B} G-protein antigen: wherein the NiV_{B} G-protein antigen comprises an amino acid sequence as set forth in SEQ ID NO: 6, or wherein the heterologous polynucleotide encodes a polypeptide coding for the NiV_{B} G-protein antigen comprising at least 90% sequence identity to the sequence as set forth in SEQ ID NO: 6, or 90% sequence identity to an nucleic acid sequence as set forth in SEQ ID NO: 2, wherein the composition or vaccine is effective to reduce or prevent a Nipah Virus infection at 3 days.
